SEQUENCE LISTING (309 sequences)

SEQ ID NO: 1 - protein, 6 aa, Artificial Sequence
  Description of Artificial Sequence Synthetic peptide
VGPAGK

SEQ ID NO: 2 - protein, 6 aa, Artificial Sequence
  Description of Artificial Sequence Synthetic peptide
GARGQA

SEQ ID NO: 3 - protein, 8 aa, Artificial Sequence
  Description of Artificial Sequence Synthetic peptide
PPGPPGPA

SEQ ID NO: 4 - protein, 8 aa, Artificial Sequence
  Description of Artificial Sequence Synthetic peptide
XRGXXGPA

SEQ ID NO: 5 - protein, 8 aa, Artificial Sequence
  Description of Artificial Sequence Synthetic peptide
DRGETGPA

SEQ ID NO: 6 - protein, 8 aa, Artificial Sequence
  Description of Artificial Sequence Synthetic peptide
RTGDAGPA

SEQ ID NO: 7 - protein, 8 aa, Artificial Sequence
  Description of Artificial Sequence Synthetic peptide
ASGPAGPA

SEQ ID NO: 8 - protein, 8 aa, Artificial Sequence
  Description of Artificial Sequence Synthetic peptide
DRGETGPA

SEQ ID NO: 9 - protein, 8 aa, Artificial Sequence
  Description of Artificial Sequence Synthetic peptide
DKGESGPA

SEQ ID NO: 10 - protein, 8 aa, Artificial Sequence
  Description of Artificial Sequence Synthetic peptide
AKGEAGPA

SEQ ID NO: 11 - protein, 8 aa, Artificial Sequence
  Description of Artificial Sequence Synthetic peptide
PPGPPGPA

SEQ ID NO: 12 - protein, 8 aa, Artificial Sequence
  Description of Artificial Sequence Synthetic peptide
EPGPPGPA

SEQ ID NO: 13 - protein, 8 aa, Artificial Sequence
  Description of Artificial Sequence Synthetic peptide
DAGPPGPA

SEQ ID NO: 14 - protein, 8 aa, Artificial Sequence
  Description of Artificial Sequence Synthetic peptide
GETGPAGA

SEQ ID NO: 15 - protein, 8 aa, Artificial Sequence
  Description of Artificial Sequence Synthetic peptide
QPSGPAGA

SEQ ID NO: 16 - protein, 8 aa, Artificial Sequence
  Description of Artificial Sequence Synthetic peptide
ERGETGPA

SEQ ID NO: 17 - protein, 8 aa, Artificial Sequence
  Description of Artificial Sequence Synthetic peptide
DRGATGPA

SEQ ID NO: 18 - protein, 8 aa, Artificial Sequence
  Description of Artificial Sequence Synthetic peptide
DRGESGPA

SEQ ID NO: 19 - protein, 8 aa, Artificial Sequence
  Description of Artificial Sequence Synthetic peptide
DPGETGPA

SEQ ID NO: 20 - protein, 7 aa, Artificial Sequence
  Description of Artificial Sequence Synthetic peptide
LNGLPGA

SEQ ID NO: 21 - protein, 8 aa, Artificial Sequence
  Description of Artificial Sequence Synthetic peptide
PSGPAGPA

SEQ ID NO: 22 - protein, 8 aa, Artificial Sequence
  Description of Artificial Sequence Synthetic peptide
PAGAAGPA

SEQ ID NO: 23 - protein, 8 aa, Artificial Sequence
  Description of Artificial Sequence Synthetic peptide
FPGARGPA

SEQ ID NO: 24 - protein, 8 aa, Artificial Sequence
  Description of Artificial Sequence Synthetic peptide
FQGLPGPA

SEQ ID NO: 25 - protein, 8 aa, Artificial Sequence
  Description of Artificial Sequence Synthetic peptide
PLGAPGPA

SEQ ID NO: 26 - protein, 8 aa, Artificial Sequence
  Description of Artificial Sequence Synthetic peptide
PPGAVGPA

SEQ ID NO: 27 - protein, 7 aa, Artificial Sequence
  Description of Artificial Sequence Synthetic peptide
MGFPGPA

SEQ ID NO: 28 - protein, 8 aa, Artificial Sequence
  Description of Artificial Sequence Synthetic peptide
RVGPPGPA

SEQ ID NO: 29 - protein, 8 aa, Artificial Sequence
  Description of Artificial Sequence Synthetic peptide
AGPVGPPA

SEQ ID NO: 30 - protein, 8 aa, Artificial Sequence
  Description of Artificial Sequence Synthetic peptide
AGPPGPPA

SEQ ID NO: 31 - protein, 8 aa, Artificial Sequence
  Description of Artificial Sequence Synthetic peptide
EPGASGPA

SEQ ID NO: 32 - protein, 8 aa, Artificial Sequence
  Description of Artificial Sequence Synthetic peptide
ETGPAGPA

SEQ ID NO: 33 - protein, 8 aa, Artificial Sequence
  Description of Artificial Sequence Synthetic peptide
PPGAVGPA

SEQ ID NO: 34 - protein, 8 aa, Artificial Sequence
  Description of Artificial Sequence Synthetic peptide
AQGPPGPA

SEQ ID NO: 35 - protein, 8 aa, Artificial Sequence
  Description of Artificial Sequence Synthetic peptide
KTGPPGPA

SEQ ID NO: 36 - protein, 8 aa, Artificial Sequence
  Description of Artificial Sequence Synthetic peptide
VMGFPGPA

SEQ ID NO: 37 - protein, 7 aa, Artificial Sequence
  Description of Artificial Sequence Synthetic peptide
SGEAGPA

SEQ ID NO: 38 - protein, 6 aa, Artificial Sequence
  Description of Artificial Sequence Synthetic peptide
XXXXXA

SEQ ID NO: 39 - protein, 6 aa, Artificial Sequence
  Description of Artificial Sequence Synthetic peptide
XXXXAG

SEQ ID NO: 40 - protein, 8 aa, Artificial Sequence
  Description of Artificial Sequence Synthetic peptide
XXXXXAGG

SEQ ID NO: 41 - protein, 5 aa, Artificial Sequence
  Description of Artificial Sequence Synthetic peptide
XXXXS

SEQ ID NO: 42 - protein, 6 aa, Artificial Sequence
  Description of Artificial Sequence Synthetic peptide
XXXXSG

SEQ ID NO: 43 - protein, 5 aa, Artificial Sequence
  Description of Artificial Sequence Synthetic peptide
XXXXV

SEQ ID NO: 44 - protein, 6 aa, Artificial Sequence
  Description of Artificial Sequence Synthetic peptide
XXXXVG

SEQ ID NO: 45 - protein, 15 aa, Artificial Sequence
  Description of Artificial Sequence Synthetic peptide
AGKDGEAGAQGPPGP

SEQ ID NO: 46 - DNA, 38 bp, Artificial Sequence
  Description of Artificial Sequence Synthetic primer
ggaagatctcatcatcaccatcaccatcgcccttcaag

SEQ ID NO: 47 - DNA, 37 bp, Artificial Sequence
  Description of Artificial Sequence Synthetic primer
ggcctcgagtcattagtctgacaaagagaaacactgc

SEQ ID NO: 48 - protein, 17 aa, Artificial Sequence
  Description of Artificial Sequence Synthetic peptide
PAGKDGEAGAQGPPGPA

SEQ ID NO: 49 - protein, 17 aa, Artificial Sequence
  Description of Artificial Sequence Synthetic peptide
PVGPPGPPGPPGPPGPP

SEQ ID NO: 50 - protein, 10 aa, Artificial Sequence
  Description of Artificial Sequence Synthetic peptide
PPGAVGPAGK

SEQ ID NO: 51 - protein, 10 aa, Artificial Sequence
  Description of Artificial Sequence Synthetic peptide
KTGPPGPAGQ

SEQ ID NO: 52 - protein, 10 aa, Artificial Sequence
  Description of Artificial Sequence Synthetic peptide
VMGFPGPKGA

SEQ ID NO: 53 - protein, 10 aa, Artificial Sequence
  Description of Artificial Sequence Synthetic peptide
VMGFPGPKGA

SEQ ID NO: 54 - protein, 10 aa, Artificial Sequence
  Description of Artificial Sequence Synthetic peptide
GFPGPKGAAG

SEQ ID NO: 55 - protein, 10 aa, Artificial Sequence
  Description of Artificial Sequence Synthetic peptide
FPGPKGAAGE

SEQ ID NO: 56 - protein, 10 aa, Artificial Sequence
  Description of Artificial Sequence Synthetic peptide
LTGSPGSPGP

SEQ ID NO: 57 - protein, 10 aa, Artificial Sequence
  Description of Artificial Sequence Synthetic peptide
KTGPPGPAGQ

SEQ ID NO: 58 - protein, 10 aa, Artificial Sequence
  Description of Artificial Sequence Synthetic peptide
VMGFPGPKGA

SEQ ID NO: 59 - protein, 10 aa, Artificial Sequence
  Description of Artificial Sequence Synthetic peptide
GLPGAKGLTG

SEQ ID NO: 60 - protein, 9 aa, Artificial Sequence
  Description of Artificial Sequence Synthetic peptide
MGFPGPKGA

SEQ ID NO: 61 - protein, 10 aa, Artificial Sequence
  Description of Artificial Sequence Synthetic peptide
PGPPGARGQA

SEQ ID NO: 62 - protein, 8 aa, Artificial Sequence
  Description of Artificial Sequence Synthetic peptide
PGARGQAG

SEQ ID NO: 63 - protein, 10 aa, Artificial Sequence
  Description of Artificial Sequence Synthetic peptide
GPPGPPGARG

SEQ ID NO: 64 - protein, 10 aa, Artificial Sequence
  Description of Artificial Sequence Synthetic peptide
PPGPPGARGQ

SEQ ID NO: 65 - protein, 10 aa, Artificial Sequence
  Description of Artificial Sequence Synthetic peptide
KTGPPGPAGQ

SEQ ID NO: 66 - protein, 10 aa, Artificial Sequence
  Description of Artificial Sequence Synthetic peptide
AQGPPGPAGP

SEQ ID NO: 67 - protein, 10 aa, Artificial Sequence
  Description of Artificial Sequence Synthetic peptide
SPGSPGPDGK

SEQ ID NO: 68 - protein, 10 aa, Artificial Sequence
  Description of Artificial Sequence Synthetic peptide
PPGPPGARGQ

SEQ ID NO: 69 - protein, 10 aa, Artificial Sequence
  Description of Artificial Sequence Synthetic peptide
PPGAVGPAGK

SEQ ID NO: 70 - protein, 10 aa, Artificial Sequence
  Description of Artificial Sequence Synthetic peptide
GEPGKAGERG

SEQ ID NO: 71 - protein, 10 aa, Artificial Sequence
  Description of Artificial Sequence Synthetic peptide
KTGPPGPAGQ

SEQ ID NO: 72 - protein, 10 aa, Artificial Sequence
  Description of Artificial Sequence Synthetic peptide
PPGAVGPAGK

SEQ ID NO: 73 - protein, 10 aa, Artificial Sequence
  Description of Artificial Sequence Synthetic peptide
PPGAVGPAGK

SEQ ID NO: 74 - protein, 10 aa, Artificial Sequence
  Description of Artificial Sequence Synthetic peptide
KTGPPGPAGQ

SEQ ID NO: 75 - protein, 10 aa, Artificial Sequence
  Description of Artificial Sequence Synthetic peptide
GPPGPPGARG

SEQ ID NO: 76 - protein, 10 aa, Artificial Sequence
  Description of Artificial Sequence Synthetic peptide
AGEPGKAGER

SEQ ID NO: 77 - protein, 10 aa, Artificial Sequence
  Description of Artificial Sequence Synthetic peptide
SPGSPGPDGK

SEQ ID NO: 78 - protein, 10 aa, Artificial Sequence
  Description of Artificial Sequence Synthetic peptide
AGEPGKAGER

SEQ ID NO: 79 - protein, 10 aa, Artificial Sequence
  Description of Artificial Sequence Synthetic peptide
VMGFPGPKGA

SEQ ID NO: 80 - protein, 10 aa, Artificial Sequence
  Description of Artificial Sequence Synthetic peptide
VMGFPGPKGA

SEQ ID NO: 81 - protein, 10 aa, Artificial Sequence
  Description of Artificial Sequence Synthetic peptide
VMGFPGPKGA

SEQ ID NO: 82 - protein, 10 aa, Artificial Sequence
  Description of Artificial Sequence Synthetic peptide
VMGFPGPKGA

SEQ ID NO: 83 - protein, 10 aa, Artificial Sequence
  Description of Artificial Sequence Synthetic peptide
GARGQAGVMG

SEQ ID NO: 84 - protein, 27 aa, Artificial Sequence
  Description of Artificial Sequence Synthetic peptide
KGAAGEPGKAGERGVPGPPGAVGPAGK

SEQ ID NO: 85 - protein, 26 aa, Artificial Sequence
  Description of Artificial Sequence Synthetic peptide
PGPKGAAGEPGKAGERGVPGPPGAVG

SEQ ID NO: 86 - protein, 26 aa, Artificial Sequence
  Description of Artificial Sequence Synthetic peptide
GPKGAAGEPGKAGERGVPGPPGAVGP

SEQ ID NO: 87 - protein, 26 aa, Artificial Sequence
  Description of Artificial Sequence Synthetic peptide
PKGAAGEPGKAGERGVPGPPGAVGPA

SEQ ID NO: 88 - protein, 26 aa, Artificial Sequence
  Description of Artificial Sequence Synthetic peptide
AAGEPGKAGERGVPGPPGAVGPAGKD

SEQ ID NO: 89 - protein, 25 aa, Artificial Sequence
  Description of Artificial Sequence Synthetic peptide
DGRPGPPGPPGARGQAGVMGFPGPK

SEQ ID NO: 90 - protein, 27 aa, Artificial Sequence
  Description of Artificial Sequence Synthetic peptide
GAVGPAGKDGEAGAQGPPGPAGPAGER

SEQ ID NO: 91 - protein, 29 aa, Artificial Sequence
  Description of Artificial Sequence Synthetic peptide
QAGVMGFPGPKGAAGEPGKAGERGVPGPP

SEQ ID NO: 92 - protein, 30 aa, Artificial Sequence
  Description of Artificial Sequence Synthetic peptide
TGSPGSPGPDGKTGPPGPAGQDGRPGPPGP

SEQ ID NO: 93 - protein, 29 aa, Artificial Sequence
  Description of Artificial Sequence Synthetic peptide
PPGARGQAGVMGFPGPKGAAGEPGKAGER

SEQ ID NO: 94 - protein, 42 aa, Artificial Sequence
  Description of Artificial Sequence Synthetic peptide
KGAAGEPGKAGERGVPGPPGAVGPAGKDGEAGAQGPPGPAGP

SEQ ID NO: 95 - protein, 41 aa, Artificial Sequence
  Description of Artificial Sequence Synthetic peptide
FPGPKGAAGEPGKAGERGVPGPPGAVGPAGKDGEAGAQGPP

SEQ ID NO: 96 - protein, 41 aa, Artificial Sequence
  Description of Artificial Sequence Synthetic peptide
PGPKGAAGEPGKAGERGVPGPPGAVGPAGKDGEAGAQGPPG

SEQ ID NO: 97 - protein, 41 aa, Artificial Sequence
  Description of Artificial Sequence Synthetic peptide
GPKGAAGEPGKAGERGVPGPPGAVGPAGKDGEAGAQGPPGP

SEQ ID NO: 98 - protein, 41 aa, Artificial Sequence
  Description of Artificial Sequence Synthetic peptide
PKGAAGEPGKAGERGVPGPPGAVGPAGKDGEAGAQGPPGPA

SEQ ID NO: 99 - protein, 40 aa, Artificial Sequence
  Description of Artificial Sequence Synthetic peptide
TGPPGPAGQDGRPGPPGPPGARGQAGVMGFPGPKGAAGEP

SEQ ID NO: 100 - protein, 21 aa, Artificial Sequence
  Description of Artificial Sequence Synthetic peptide
PPGPAGQDGRPGPPGPPGARG

SEQ ID NO: 101 - protein, 19 aa, Artificial Sequence
  Description of Artificial Sequence Synthetic peptide
TGFPGAAGRVGPPGPSGNA

SEQ ID NO: 102 - protein, 18 aa, Artificial Sequence
  Description of Artificial Sequence Synthetic peptide
GPPGPAGPAGPPGPIGNV

SEQ ID NO: 103 - protein, 24 aa, Artificial Sequence
  Description of Artificial Sequence Synthetic peptide
GETGPAGPPGAPGAPGAPGPVGPA

SEQ ID NO: 104 - protein, 34 aa, Artificial Sequence
  Description of Artificial Sequence Synthetic peptide
AGPPGAPGAPGAPGPVGPAGKSGDRGETGPAGPA

SEQ ID NO: 105 - protein, 31 aa, Artificial Sequence
  Description of Artificial Sequence Synthetic peptide
PGAPGAPGAPGPVGPAGKSGDRGETGPAGPA

SEQ ID NO: 106 - protein, 19 aa, Artificial Sequence
  Description of Artificial Sequence Synthetic peptide
PGPAGAPGDKGESGPSGPA

SEQ ID NO: 107 - protein, 28 aa, Artificial Sequence
  Description of Artificial Sequence Synthetic peptide
PGAPGAPGPVGPAGKSGDRGETGPAGPA

SEQ ID NO: 108 - protein, 33 aa, Artificial Sequence
  Description of Artificial Sequence Synthetic peptide
GPPGADGQPGAKGEPGDAGAKGDAGPPGPAGPA

SEQ ID NO: 109 - protein, 26 aa, Artificial Sequence
  Description of Artificial Sequence Synthetic peptide
GPAGQDGRPGPPGPPGARGQAGVMGF

SEQ ID NO: 110 - protein, 28 aa, Artificial Sequence
  Description of Artificial Sequence Synthetic peptide
PGPSGEPGKQGPSGASGERGPPGPMGPP

SEQ ID NO: 111 - protein, 38 aa, Artificial Sequence
  Description of Artificial Sequence Synthetic peptide
PAGFAGPPGADGQPGAKGEPGDAGAKGDAGPPGPAGPA

SEQ ID NO: 112 - protein, 38 aa, Artificial Sequence
  Description of Artificial Sequence Synthetic peptide
ETGPAGPPGAPGAPGAPGPVGPAGKSGDRGETGPAGPA

SEQ ID NO: 113 - protein, 21 aa, Artificial Sequence
  Description of Artificial Sequence Synthetic peptide
LTGPIGPPGPAGAPGDKGESG

SEQ ID NO: 114 - protein, 26 aa, Artificial Sequence
  Description of Artificial Sequence Synthetic peptide
LTGPIGPPGPAGAPGDKGESGPSGPA

SEQ ID NO: 115 - protein, 23 aa, Artificial Sequence
  Description of Artificial Sequence Synthetic peptide
AKGDAGPPGPAGPAGPPGPIGNV

SEQ ID NO: 116 - protein, 32 aa, Artificial Sequence
  Description of Artificial Sequence Synthetic peptide
FPGLPGPSGEPGKQGPSGASGERGPPGPMGPP

SEQ ID NO: 117 - protein, 29 aa, Artificial Sequence
  Description of Artificial Sequence Synthetic peptide
PSGPAGPTGARGAPGDRGEPGPPGPAGFA

SEQ ID NO: 118 - protein, 31 aa, Artificial Sequence
  Description of Artificial Sequence Synthetic peptide
PGPPGPAGEKGSPGADGPAGAPGTPGPQGIA

SEQ ID NO: 119 - protein, 43 aa, Artificial Sequence
  Description of Artificial Sequence Synthetic peptide
PGPPGPAGFAGPPGADGQPGAKGEPGDAGAKGDAGPPGPAGPA

SEQ ID NO: 120 - protein, 25 aa, Artificial Sequence
  Description of Artificial Sequence Synthetic peptide
PGAVGPAGKDGEAGAQGPPGPAGPA

SEQ ID NO: 121 - protein, 43 aa, Artificial Sequence
  Description of Artificial Sequence Synthetic peptide
AGAAGPAGNPGADGQPGAKGANGAPGIAGAPGFPGARGPSGPQ

SEQ ID NO: 122 - protein, 27 aa, Artificial Sequence
  Description of Artificial Sequence Synthetic peptide
KGDAGPAGPKGEPGSPGENGAPGQMGP

SEQ ID NO: 123 - protein, 25 aa, Artificial Sequence
  Description of Artificial Sequence Synthetic peptide
RGETGPAGPPGAPGAPGAPGPVGPA

SEQ ID NO: 124 - protein, 28 aa, Artificial Sequence
  Description of Artificial Sequence Synthetic peptide
RGETGPAGPPGAPGAPGAPGPVGPAGKS

SEQ ID NO: 125 - protein, 40 aa, Artificial Sequence
  Description of Artificial Sequence Synthetic peptide
RGETGPAGPPGAPGAPGAPGPVGPAGKSGDRGETGPAGPA

SEQ ID NO: 126 - protein, 34 aa, Artificial Sequence
  Description of Artificial Sequence Synthetic peptide
QGLPGPAGPPGEAGKPGEQGVPGDLGAPGPSGAR

SEQ ID NO: 127 - protein, 28 aa, Artificial Sequence
  Description of Artificial Sequence Synthetic peptide
PGPTGPAGPPGFPGAVGAKGEAGPQGPR

SEQ ID NO: 128 - protein, 49 aa, Artificial Sequence
  Description of Artificial Sequence Synthetic peptide
PGPTGPAGPPGFPGAVGAKGEAGPQGPRGSEGPQGVRGEPGPPGPAGAA

SEQ ID NO: 129 - protein, 52 aa, Artificial Sequence
  Description of Artificial Sequence Synthetic peptide
PGPAGPAGPPGPIGNVGAPGAKGARGSAGPPGATGFPGAAGRVGPPGPSGNA

SEQ ID NO: 130 - protein, 31 aa, Artificial Sequence
  Description of Artificial Sequence Synthetic peptide
SGPSGPAGPTGARGAPGDRGEPGPPGPAGFA

SEQ ID NO: 131 - protein, 30 aa, Artificial Sequence
  Description of Artificial Sequence Synthetic peptide
TGPPGPAGQDGRPGPPGPPGARGQAGVMGF

SEQ ID NO: 132 - protein, 25 aa, Artificial Sequence
  Description of Artificial Sequence Synthetic peptide
RGETGPAGRPGEVGPPGPPGPAGEK

SEQ ID NO: 133 - protein, 31 aa, Artificial Sequence
  Description of Artificial Sequence Synthetic peptide
SGPQGPGGPPGPKGNSGEPGAPGSKGDTGAK

SEQ ID NO: 134 - protein, 18 aa, Artificial Sequence
  Description of Artificial Sequence Synthetic peptide
GPRGLPGPPGPAGPQGFQ

SEQ ID NO: 135 - protein, 22 aa, Artificial Sequence
  Description of Artificial Sequence Synthetic peptide
GRVGPPGPSGNAGPPGPPGPAG

SEQ ID NO: 136 - protein, 28 aa, Artificial Sequence
  Description of Artificial Sequence Synthetic peptide
RGLTGPIGPPGPAGAPGDKGESGPSGPA

SEQ ID NO: 137 - protein, 34 aa, Artificial Sequence
  Description of Artificial Sequence Synthetic peptide
MGFPGPKGAAGEPGKAGERGVPGPPGAVGPAGKD

SEQ ID NO: 138 - protein, 49 aa, Artificial Sequence
  Description of Artificial Sequence Synthetic peptide
MGFPGPKGAAGEPGKAGERGVPGPPGAVGPAGKDGEAGAQGPPGPAGPA

SEQ ID NO: 139 - protein, 37 aa, Artificial Sequence
  Description of Artificial Sequence Synthetic peptide
TGPAGPPGAPGAPGAPGPVGPAGKSGDRGETGPAGPA

SEQ ID NO: 140 - protein, 35 aa, Artificial Sequence
  Description of Artificial Sequence Synthetic peptide
PGPMGPPGLAGPPGESGREGAPGAEGSPGRDGSPG

SEQ ID NO: 141 - protein, 25 aa, Artificial Sequence
  Description of Artificial Sequence Synthetic peptide
TGPIGPPGPAGAPGDKGESGPSGPA

SEQ ID NO: 142 - protein, 28 aa, Artificial Sequence
  Description of Artificial Sequence Synthetic peptide
PGAPGPQGFQGPPGEPGEPGASGPMGPR

SEQ ID NO: 143 - protein, 22 aa, Artificial Sequence
  Description of Artificial Sequence Synthetic peptide
RGSEGPQGVRGEPGPPGPAGAA

SEQ ID NO: 144 - protein, 31 aa, Artificial Sequence
  Description of Artificial Sequence Synthetic peptide
SGPAGPRGPPGSAGAPGKDGLNGLPGPIGPP

SEQ ID NO: 145 - protein, 31 aa, Artificial Sequence
  Description of Artificial Sequence Synthetic peptide
PGLPGPSGEPGKQGPSGASGERGPPGPMGPP

SEQ ID NO: 146 - protein, 22 aa, Artificial Sequence
  Description of Artificial Sequence Synthetic peptide
VGPPGPSGNAGPPGPPGPAGKE

SEQ ID NO: 147 - protein, 28 aa, Artificial Sequence
  Description of Artificial Sequence Synthetic peptide
SGPAGPTGARGAPGDRGEPGPPGPAGFA

SEQ ID NO: 148 - protein, 20 aa, Artificial Sequence
  Description of Artificial Sequence Synthetic peptide
TGDAGPVGPPGPPGPPGPPG

SEQ ID NO: 149 - protein, 22 aa, Artificial Sequence
  Description of Artificial Sequence Synthetic peptide
TGDAGPVGPPGPPGPPGPPGPP

SEQ ID NO: 150 - protein, 19 aa, Artificial Sequence
  Description of Artificial Sequence Synthetic peptide
KGEPGPVGVQGPPGPAGEE

SEQ ID NO: 151 - protein, 19 aa, Artificial Sequence
  Description of Artificial Sequence Synthetic peptide
GDAGPVGPPGPPGPPGPPG

SEQ ID NO: 152 - protein, 6 aa, Artificial Sequence
  Description of Artificial Sequence Synthetic 6xHis tag
HHHHHH

SEQ ID NO: 153 - protein, 10 aa, Artificial Sequence
  Description of Artificial Sequence Synthetic peptide
PPGAVGPAGK

SEQ ID NO: 154 - protein, 10 aa, Artificial Sequence
  Description of Artificial Sequence Synthetic peptide
KTGPPGPAGQ

SEQ ID NO: 155 - protein, 10 aa, Artificial Sequence
  Description of Artificial Sequence Synthetic peptide
VMGFPGPKGA

SEQ ID NO: 156 - protein, 10 aa, Artificial Sequence
  Description of Artificial Sequence Synthetic peptide
VMGFPGPKGA

SEQ ID NO: 157 - protein, 10 aa, Artificial Sequence
  Description of Artificial Sequence Synthetic peptide
GFPGPKGAAG

SEQ ID NO: 158 - protein, 10 aa, Artificial Sequence
  Description of Artificial Sequence Synthetic peptide
FPGPKGAAGE

SEQ ID NO: 159 - protein, 10 aa, Artificial Sequence
  Description of Artificial Sequence Synthetic peptide
LTGSPGSPGP

SEQ ID NO: 160 - protein, 10 aa, Artificial Sequence
  Description of Artificial Sequence Synthetic peptide
KTGPPGPAGQ

SEQ ID NO: 161 - protein, 10 aa, Artificial Sequence
  Description of Artificial Sequence Synthetic peptide
VMGFPGPKGA

SEQ ID NO: 162 - protein, 10 aa, Artificial Sequence
  Description of Artificial Sequence Synthetic peptide
GLPGAKGLTG

SEQ ID NO: 163 - protein, 9 aa, Artificial Sequence
  Description of Artificial Sequence Synthetic peptide
MGFPGPKGA

SEQ ID NO: 164 - protein, 10 aa, Artificial Sequence
  Description of Artificial Sequence Synthetic peptide
PGPPGARGQA

SEQ ID NO: 165 - protein, 8 aa, Artificial Sequence
  Description of Artificial Sequence Synthetic peptide
PGARGQAG

SEQ ID NO: 166 - protein, 10 aa, Artificial Sequence
  Description of Artificial Sequence Synthetic peptide
GPPGPPGARG

SEQ ID NO: 167 - protein, 10 aa, Artificial Sequence
  Description of Artificial Sequence Synthetic peptide
PPGPPGARGQ

SEQ ID NO: 168 - protein, 10 aa, Artificial Sequence
  Description of Artificial Sequence Synthetic peptide
KTGPPGPAGQ

SEQ ID NO: 169 - protein, 10 aa, Artificial Sequence
  Description of Artificial Sequence Synthetic peptide
AQGPPGPAGP

SEQ ID NO: 170 - protein, 10 aa, Artificial Sequence
  Description of Artificial Sequence Synthetic peptide
SPGSPGPDGK

SEQ ID NO: 171 - protein, 10 aa, Artificial Sequence
  Description of Artificial Sequence Synthetic peptide
PPGPPGARGQ

SEQ ID NO: 172 - protein, 10 aa, Artificial Sequence
  Description of Artificial Sequence Synthetic peptide
PPGAVGPAGK

SEQ ID NO: 173 - protein, 10 aa, Artificial Sequence
  Description of Artificial Sequence Synthetic peptide
GEPGKAGERG

SEQ ID NO: 174 - protein, 10 aa, Artificial Sequence
  Description of Artificial Sequence Synthetic peptide
KTGPPGPAGQ

SEQ ID NO: 175 - protein, 10 aa, Artificial Sequence
  Description of Artificial Sequence Synthetic peptide
PPGAVGPAGK

SEQ ID NO: 176 - protein, 10 aa, Artificial Sequence
  Description of Artificial Sequence Synthetic peptide
PPGAVGPAGK

SEQ ID NO: 177 - protein, 10 aa, Artificial Sequence
  Description of Artificial Sequence Synthetic peptide
KTGPPGPAGQ

SEQ ID NO: 178 - protein, 10 aa, Artificial Sequence
  Description of Artificial Sequence Synthetic peptide
GPPGPPGARG

SEQ ID NO: 179 - protein, 10 aa, Artificial Sequence
  Description of Artificial Sequence Synthetic peptide
AGEPGKAGER

SEQ ID NO: 180 - protein, 10 aa, Artificial Sequence
  Description of Artificial Sequence Synthetic peptide
SPGSPGPDGK

SEQ ID NO: 181 - protein, 10 aa, Artificial Sequence
  Description of Artificial Sequence Synthetic peptide
AGEPGKAGER

SEQ ID NO: 182 - protein, 10 aa, Artificial Sequence
  Description of Artificial Sequence Synthetic peptide
VMGFPGPKGA

SEQ ID NO: 183 - protein, 10 aa, Artificial Sequence
  Description of Artificial Sequence Synthetic peptide
VMGFPGPKGA

SEQ ID NO: 184 - protein, 10 aa, Artificial Sequence
  Description of Artificial Sequence Synthetic peptide
VMGFPGPKGA

SEQ ID NO: 185 - protein, 10 aa, Artificial Sequence
  Description of Artificial Sequence Synthetic peptide
VMGFPGPKGA

SEQ ID NO: 186 - protein, 10 aa, Artificial Sequence
  Description of Artificial Sequence Synthetic peptide
GARGQAGVMG

SEQ ID NO: 187 - protein, 10 aa, Artificial Sequence
  Description of Artificial Sequence Synthetic peptide
RTGDAGPVGP

SEQ ID NO: 188 - protein, 10 aa, Artificial Sequence
  Description of Artificial Sequence Synthetic peptide
ASGPAGPRGP

SEQ ID NO: 189 - protein, 10 aa, Artificial Sequence
  Description of Artificial Sequence Synthetic peptide
DRGETGPAGP

SEQ ID NO: 190 - protein, 10 aa, Artificial Sequence
  Description of Artificial Sequence Synthetic peptide
DRGETGPAGP

SEQ ID NO: 191 - protein, 10 aa, Artificial Sequence
  Description of Artificial Sequence Synthetic peptide
EPGPPGPAGF

SEQ ID NO: 192 - protein, 10 aa, Artificial Sequence
  Description of Artificial Sequence Synthetic peptide
RTGDAGPVGP

SEQ ID NO: 193 - protein, 10 aa, Artificial Sequence
  Description of Artificial Sequence Synthetic peptide
GETGPAGPPG

SEQ ID NO: 194 - protein, 10 aa, Artificial Sequence
  Description of Artificial Sequence Synthetic peptide
QPSGPAGPTG

SEQ ID NO: 195 - protein, 10 aa, Artificial Sequence
  Description of Artificial Sequence Synthetic peptide
PSGPAGPTGA

SEQ ID NO: 196 - protein, 10 aa, Artificial Sequence
  Description of Artificial Sequence Synthetic peptide
PAGAAGPAGN

SEQ ID NO: 197 - protein, 10 aa, Artificial Sequence
  Description of Artificial Sequence Synthetic peptide
PAGPPGAPGA

SEQ ID NO: 198 - protein, 10 aa, Artificial Sequence
  Description of Artificial Sequence Synthetic peptide
FQGLPGPAGP

SEQ ID NO: 199 - protein, 9 aa, Artificial Sequence
  Description of Artificial Sequence Synthetic peptide
MGFPGPKGA

SEQ ID NO: 200 - protein, 10 aa, Artificial Sequence
  Description of Artificial Sequence Synthetic peptide
PPGPAGPAGP

SEQ ID NO: 201 - protein, 10 aa, Artificial Sequence
  Description of Artificial Sequence Synthetic peptide
RVGPPGPSGN

SEQ ID NO: 202 - protein, 10 aa, Artificial Sequence
  Description of Artificial Sequence Synthetic peptide
AGRVGPPGPS

SEQ ID NO: 203 - protein, 10 aa, Artificial Sequence
  Description of Artificial Sequence Synthetic peptide
PPGAPGPQGF

SEQ ID NO: 204 - protein, 10 aa, Artificial Sequence
  Description of Artificial Sequence Synthetic peptide
ETGPAGPPGA

SEQ ID NO: 205 - protein, 10 aa, Artificial Sequence
  Description of Artificial Sequence Synthetic peptide
PPGAPGAPGA

SEQ ID NO: 206 - protein, 10 aa, Artificial Sequence
  Description of Artificial Sequence Synthetic peptide
GLTGPIGPPG

SEQ ID NO: 207 - protein, 10 aa, Artificial Sequence
  Description of Artificial Sequence Synthetic peptide
DRGETGPAGP

SEQ ID NO: 208 - protein, 10 aa, Artificial Sequence
  Description of Artificial Sequence Synthetic peptide
ESGPSGPAGP

SEQ ID NO: 209 - protein, 10 aa, Artificial Sequence
  Description of Artificial Sequence Synthetic peptide
PRGETGPAGR

SEQ ID NO: 210 - protein, 10 aa, Artificial Sequence
  Description of Artificial Sequence Synthetic peptide
VRGLTGPIGP

SEQ ID NO: 211 - protein, 10 aa, Artificial Sequence
  Description of Artificial Sequence Synthetic peptide
PPGPTGPAGP

SEQ ID NO: 212 - protein, 10 aa, Artificial Sequence
  Description of Artificial Sequence Synthetic peptide
FPGLPGPSGE

SEQ ID NO: 213 - protein, 6 aa, Artificial Sequence
  Description of Artificial Sequence Synthetic peptide
PGPPGP

SEQ ID NO: 214 - protein, 10 aa, Artificial Sequence
  Description of Artificial Sequence Synthetic peptide
LNGLPGPIGP

SEQ ID NO: 215 - protein, 10 aa, Artificial Sequence
  Description of Artificial Sequence Synthetic peptide
APGAPGPVGP

SEQ ID NO: 216 - protein, 10 aa, Artificial Sequence
  Description of Artificial Sequence Synthetic peptide
DRGETGPAGP

SEQ ID NO: 217 - protein, 10 aa, Artificial Sequence
  Description of Artificial Sequence Synthetic peptide
DAGPPGPAGP

SEQ ID NO: 218 - protein, 10 aa, Artificial Sequence
  Description of Artificial Sequence Synthetic peptide
PGPPGPPGPP

SEQ ID NO: 219 - protein, 10 aa, Artificial Sequence
  Description of Artificial Sequence Synthetic peptide
DRGETGPAGP

SEQ ID NO: 220 - protein, 10 aa, Artificial Sequence
  Description of Artificial Sequence Synthetic peptide
EPGPPGPAGF

SEQ ID NO: 221 - protein, 10 aa, Artificial Sequence
  Description of Artificial Sequence Synthetic peptide
EPGPPGPAGF

SEQ ID NO: 222 - protein, 10 aa, Artificial Sequence
  Description of Artificial Sequence Synthetic peptide
FPGARGPSGP

SEQ ID NO: 223 - protein, 10 aa, Artificial Sequence
  Description of Artificial Sequence Synthetic peptide
DRGETGPAGP

SEQ ID NO: 224 - protein, 10 aa, Artificial Sequence
  Description of Artificial Sequence Synthetic peptide
DLGAPGPSGA

SEQ ID NO: 225 - protein, 10 aa, Artificial Sequence
  Description of Artificial Sequence Synthetic peptide
PPGAVGPAGK

SEQ ID NO: 226 - protein, 10 aa, Artificial Sequence
  Description of Artificial Sequence Synthetic peptide
RVGPPGPSGN

SEQ ID NO: 227 - protein, 10 aa, Artificial Sequence
  Description of Artificial Sequence Synthetic peptide
PPGPPGPAGK

SEQ ID NO: 228 - protein, 10 aa, Artificial Sequence
  Description of Artificial Sequence Synthetic peptide
AGPPGPPGPA

SEQ ID NO: 229 - protein, 10 aa, Artificial Sequence
  Description of Artificial Sequence Synthetic peptide
EPGASGPMGP

SEQ ID NO: 230 - protein, 10 aa, Artificial Sequence
  Description of Artificial Sequence Synthetic peptide
DRGETGPAGP

SEQ ID NO: 231 - protein, 10 aa, Artificial Sequence
  Description of Artificial Sequence Synthetic peptide
DRGETGPAGP

SEQ ID NO: 232 - protein, 10 aa, Artificial Sequence
  Description of Artificial Sequence Synthetic peptide
DKGESGPSGP

SEQ ID NO: 233 - protein, 10 aa, Artificial Sequence
  Description of Artificial Sequence Synthetic peptide
APGPVGPAGK

SEQ ID NO: 234 - protein, 10 aa, Artificial Sequence
  Description of Artificial Sequence Synthetic peptide
EPGPPGPAGF

SEQ ID NO: 235 - protein, 10 aa, Artificial Sequence
  Description of Artificial Sequence Synthetic peptide
PPGPPGPAGE

SEQ ID NO: 236 - protein, 10 aa, Artificial Sequence
  Description of Artificial Sequence Synthetic peptide
DKGESGPSGP

SEQ ID NO: 237 - protein, 10 aa, Artificial Sequence
  Description of Artificial Sequence Synthetic peptide
AKGEAGPQGP

SEQ ID NO: 238 - protein, 10 aa, Artificial Sequence
  Description of Artificial Sequence Synthetic peptide
ERGPPGPMGP

SEQ ID NO: 239 - protein, 10 aa, Artificial Sequence
  Description of Artificial Sequence Synthetic peptide
PPGPPGPAGE

SEQ ID NO: 240 - protein, 10 aa, Artificial Sequence
  Description of Artificial Sequence Synthetic peptide
LTGPIGPPGP

SEQ ID NO: 241 - protein, 10 aa, Artificial Sequence
  Description of Artificial Sequence Synthetic peptide
AKGDAGPAGP

SEQ ID NO: 242 - protein, 10 aa, Artificial Sequence
  Description of Artificial Sequence Synthetic peptide
VMGFPGPKGA

SEQ ID NO: 243 - protein, 9 aa, Artificial Sequence
  Description of Artificial Sequence Synthetic peptide
PPGPAGAPG

SEQ ID NO: 244 - protein, 10 aa, Artificial Sequence
  Description of Artificial Sequence Synthetic peptide
PPGPMGPPGL

SEQ ID NO: 245 - protein, 10 aa, Artificial Sequence
  Description of Artificial Sequence Synthetic peptide
GFPGLPGPSG

SEQ ID NO: 246 - protein, 10 aa, Artificial Sequence
  Description of Artificial Sequence Synthetic peptide
PPGPTGPAGP

SEQ ID NO: 247 - protein, 10 aa, Artificial Sequence
  Description of Artificial Sequence Synthetic peptide
APGAPGAPGP

SEQ ID NO: 248 - protein, 10 aa, Artificial Sequence
  Description of Artificial Sequence Synthetic peptide
PRGSEGPQGV

SEQ ID NO: 249 - protein, 9 aa, Artificial Sequence
  Description of Artificial Sequence Synthetic peptide
TGDAGPVGP

SEQ ID NO: 250 - protein, 10 aa, Artificial Sequence
  Description of Artificial Sequence Synthetic peptide
GPAGFAGPPG

SEQ ID NO: 251 - protein, 9 aa, Artificial Sequence
  Description of Artificial Sequence Synthetic peptide
AKGEPGPVG

SEQ ID NO: 252 - protein, 10 aa, Artificial Sequence
  Description of Artificial Sequence Synthetic peptide
AGPPGADGQP

SEQ ID NO: 253 - protein, 10 aa, Artificial Sequence
  Description of Artificial Sequence Synthetic peptide
LPGPSGEPGK

SEQ ID NO: 254 - protein, 10 aa, Artificial Sequence
  Description of Artificial Sequence Synthetic peptide
GLTGPIGPPG

SEQ ID NO: 255 - protein, 10 aa, Artificial Sequence
  Description of Artificial Sequence Synthetic peptide
KTGPPGPAGQ

SEQ ID NO: 256 - protein, 10 aa, Artificial Sequence
  Description of Artificial Sequence Synthetic peptide
GAKGDAGPPG

SEQ ID NO: 257 - protein, 10 aa, Artificial Sequence
  Description of Artificial Sequence Synthetic peptide
RGETGPAGPP

SEQ ID NO: 258 - protein, 9 aa, Artificial Sequence
  Description of Artificial Sequence Synthetic peptide
ATGFPGAAG

SEQ ID NO: 259 - protein, 10 aa, Artificial Sequence
  Description of Artificial Sequence Synthetic peptide
PGPAGQDGRP

SEQ ID NO: 260 - protein, 8 aa, Artificial Sequence
  Description of Artificial Sequence Synthetic peptide
SGPRGLPG

SEQ ID NO: 261 - protein, 10 aa, Artificial Sequence
  Description of Artificial Sequence Synthetic peptide
PPGAVGPAGK

SEQ ID NO: 262 - protein, 8 aa, Artificial Sequence
  Description of Artificial Sequence Synthetic peptide
AGPPGPAG

SEQ ID NO: 263 - protein, 10 aa, Artificial Sequence
  Description of Artificial Sequence Synthetic peptide
PSGPQGPGGP

SEQ ID NO: 264 - protein, 10 aa, Artificial Sequence
  Description of Artificial Sequence Synthetic peptide
APGTPGPQGI

SEQ ID NO: 265 - protein, 10 aa, Artificial Sequence
  Description of Artificial Sequence Synthetic peptide
DKGESGPSGP

SEQ ID NO: 266 - protein, 10 aa, Artificial Sequence
  Description of Artificial Sequence Synthetic peptide
GENGAPGQMG

SEQ ID NO: 267 - protein, 10 aa, Artificial Sequence
  Description of Artificial Sequence Synthetic peptide
AQGPPGPAGP

SEQ ID NO: 268 - protein, 10 aa, Artificial Sequence
  Description of Artificial Sequence Synthetic peptide
DKGESGPSGP

SEQ ID NO: 269 - protein, 10 aa, Artificial Sequence
  Description of Artificial Sequence Synthetic peptide
EGSPGRDGSP

SEQ ID NO: 270 - protein, 10 aa, Artificial Sequence
  Description of Artificial Sequence Synthetic peptide
ERGPPGPMGP

SEQ ID NO: 271 - protein, 10 aa, Artificial Sequence
  Description of Artificial Sequence Synthetic peptide
EPGPPGPAGA

SEQ ID NO: 272 - protein, 10 aa, Artificial Sequence
  Description of Artificial Sequence Synthetic peptide
DRGETGPAGP

SEQ ID NO: 273 - protein, 10 aa, Artificial Sequence
  Description of Artificial Sequence Synthetic peptide
EPGPPGPAGA

SEQ ID NO: 274 - protein, 10 aa, Artificial Sequence
  Description of Artificial Sequence Synthetic peptide
PGPPGPPGPP

SEQ ID NO: 275 - protein, 10 aa, Artificial Sequence
  Description of Artificial Sequence Synthetic peptide
DAGPPGPAGP

SEQ ID NO: 276 - protein, 10 aa, Artificial Sequence
  Description of Artificial Sequence Synthetic peptide
VQGPPGPAGE

SEQ ID NO: 277 - protein, 10 aa, Artificial Sequence
  Description of Artificial Sequence Synthetic peptide
DAGPPGPAGP

SEQ ID NO: 278 - protein, 10 aa, Artificial Sequence
  Description of Artificial Sequence Synthetic peptide
ERGPPGPMGP

SEQ ID NO: 279 - protein, 10 aa, Artificial Sequence
  Description of Artificial Sequence Synthetic peptide
AGAPGDKGES

SEQ ID NO: 280 - protein, 10 aa, Artificial Sequence
  Description of Artificial Sequence Synthetic peptide
GARGQAGVMG

SEQ ID NO: 281 - protein, 10 aa, Artificial Sequence
  Description of Artificial Sequence Synthetic peptide
PAGPPGPIGN

SEQ ID NO: 282 - protein, 10 aa, Artificial Sequence
  Description of Artificial Sequence Synthetic peptide
APGAPGPVGP

SEQ ID NO: 283 - protein, 10 aa, Artificial Sequence
  Description of Artificial Sequence Synthetic peptide
RVGPPGPSGN

SEQ ID NO: 284 - protein, 10 aa, Artificial Sequence
  Description of Artificial Sequence Synthetic peptide
GARGQAGVMG

SEQ ID NO: 285 - protein, 10 aa, Artificial Sequence
  Description of Artificial Sequence Synthetic peptide
PPGAPGPQGF

SEQ ID NO: 286 - protein, 10 aa, Artificial Sequence
  Description of Artificial Sequence Synthetic peptide
AQGPPGPAGP

SEQ ID NO: 287 - protein, 10 aa, Artificial Sequence
  Description of Artificial Sequence Synthetic peptide
PAGPPGPIGN

SEQ ID NO: 288 - protein, 10 aa, Artificial Sequence
  Description of Artificial Sequence Synthetic peptide
APGSKGDTGA

SEQ ID NO: 289 - protein, 5 aa, Artificial Sequence
  Description of Artificial Sequence Synthetic 5xHis tag
HHHHH

SEQ ID NO: 290 - protein, 4 aa, Artificial Sequence
  Description of Artificial Sequence Synthetic peptide
PAGP

SEQ ID NO: 291 - protein, 6 aa, Artificial Sequence
  Description of Artificial Sequence Synthetic peptide
VGPAGK

SEQ ID NO: 292 - protein, 6 aa, Artificial Sequence
  Description of Artificial Sequence Synthetic peptide
PGPAGQ

SEQ ID NO: 293 - protein, 6 aa, Artificial Sequence
  Description of Artificial Sequence Synthetic peptide
VGPAGK

SEQ ID NO: 294 - protein, 6 aa, Artificial Sequence
  Description of Artificial Sequence Synthetic peptide
PGPKGA

SEQ ID NO: 295 - protein, 6 aa, Artificial Sequence
  Description of Artificial Sequence Synthetic peptide
GAVGPA

SEQ ID NO: 296 - protein, 6 aa, Artificial Sequence
  Description of Artificial Sequence Synthetic peptide
GARGQA

SEQ ID NO: 297 - protein, 4 aa, Artificial Sequence
  Description of Artificial Sequence Synthetic peptide
PAGP

SEQ ID NO: 298 - protein, 6 aa, Artificial Sequence
  Description of Artificial Sequence Synthetic peptide
KGAAGE

SEQ ID NO: 299 - protein, 6 aa, Artificial Sequence
  Description of Artificial Sequence Synthetic peptide
PGKAGE

SEQ ID NO: 300 - protein, 8 aa, Artificial Sequence
  Description of Artificial Sequence Synthetic peptide
ERGETGPA

SEQ ID NO: 301 - protein, 8 aa, Artificial Sequence
  Description of Artificial Sequence Synthetic peptide
ASGPAGPA

SEQ ID NO: 302 - protein, 8 aa, Artificial Sequence
  Description of Artificial Sequence Synthetic peptide
EPGPPGPA

SEQ ID NO: 303 - protein, 8 aa, Artificial Sequence
  Description of Artificial Sequence Synthetic peptide
EKGESGPA

SEQ ID NO: 304 - protein, 8 aa, Artificial Sequence
  Description of Artificial Sequence Synthetic peptide
APGSKGEA

SEQ ID NO: 305 - protein, 9 aa, Artificial Sequence
  Description of Artificial Sequence Synthetic peptide
ERGETGPAG

SEQ ID NO: 306 - protein, 10 aa, Artificial Sequence
  Description of Artificial Sequence Synthetic peptide
ERGETGPAGG

SEQ ID NO: 307 - protein, 9 aa, Artificial Sequence
  Description of Artificial Sequence Synthetic peptide
ERGETGPSG

SEQ ID NO: 308 - protein, 4 aa, Artificial Sequence
  Description of Artificial Sequence Synthetic peptide
PAGP

SEQ ID NO: 309 - protein, 6 aa, Artificial Sequence
  Description of Artificial Sequence Synthetic peptide
GARGQA

(19) United States
(12) Patent Application Publication
(10) Pub. No.: US 2010/0047170 A1
(43) Pub. Date: Feb. 25, 2010

(54) Peptide Prodrugs

(76) Inventors: Samuel R. Denmeade, Ellicot City, MD (US); Saurabh Aggarwal, Baltimore, MD (US)

Correspondence Address: EDWARDS ANGELL PALMER & DODGE LLP, P.O. BOX 55874, BOSTON, MA, 02205, US

(21) Appl. No.: 12/087,398

(22) PCT Filed: Jan. 5, 2007
(86) PCT No.: PCT/US2007/000185

§ 371 Date: Mar. 27, 2009

Related U.S. Application Data
(60) Provisional application No. 60/756,358, filed on Jan. 5, 2006.

(51) Int. Cl. A61K 49/00; G01N 33/53; C12P 21/06; C12N 5/10; A61K 38/08; C07K 7/06

(52) U.S. Cl. 424/9.1; 435/7.1; 435/69.1; 435/325; 514/16; 514/17; 530/328; 530/329; 530/330; 530/387.3; 536/23.1

(57) ABSTRACT

Provided herein are a novel class of oligopeptides and prodrugs that include amino acid sequences containing cleavage sites for fibroblast activation protein (FAP). Also provided herein are methods of treating FAP related disorders, including cancer.

### RELATED APPLICATIONS

This application claims the benefit of U.S. Provisional Application No. 60/756,358, filed on Jan. 5, 2006, which is incorporated herein by reference in its entirety.

### BACKGROUND

Fibroblast activation protein (FAP, also known as seprase) is a cell surface serine protease expressed at sites of tissue remodeling in embryonic development. FAP is not expressed by mature somatic tissues except activated melanocytes and fibroblasts in wound healing or tumor stroma. FAP expression is specifically silenced in proliferating melanocytic cells during malignant transformation (Ramirez-Montagut et al (2004) Oncogene 23(32):5435-5446). FAP belongs to the prolyl peptidase family, which comprises serine proteases that cleave peptide substrates after a proline residue (Rosenblum et al (2003) Current Opinion in Chemical Biology 7(4):496-504; Sedo et al (2001) Biochimica et biophysica acta 1550(2): 107-116; Busek et al (2004) Intl. Jour. of Biochem. & Cell Biol. 36:408-421). The prolyl peptidase family also includes dipeptidyl peptidase IV (DPP IV; also termed CD26), DPP7 (DPP II; quiescent cell proline dipeptidase), DPP8, DPP9, and prolyl caiboxypeptidase (PCP; angiotensinase C). More distant members include prolyl oligopeptidase (POP or prolyl endopeptidase (PEP); post-proline cleaving enzyme; Ito, K. et al (2004) Editor(s): Barrett, Rawlings, Woessner, Handbook of Proteolytic Enzymes (2nd Edition) 2:1897-1900, Elsevier, London, UK; Polgar, L. (2002) Cellular and Molecular Life Sciences 59, 349-362) and acylaminoacylpeptidase (AAP; acylpeptide hydrolase (APH)). Proline peptidases and related proteins contain both membrane-bound and soluble members and span a broad range of expression patterns, tissue distributions and compartmentalization. These proteins have important roles in regulation of signaling by peptide hormones, and are emerging targets for diabetes, oncology, and other indications.

Metastatic epithelial cancers are composed of heterogeneous populations of cells that can have variable response to antitumor agents. Currently utilized standard antiproliferative chemotherapies can produce modest improvement in survival in select cancer types. However, for the most part, epithelial cancers remain largely incurable once they have escaped their organ of origin. Novel therapies for metastatic cancer, therefore, are needed. Thus, there is a need in the art for compounds targeting FAP for treatment of serine protease related disorders, e.g., epithelial cancers and inflammatory conditions (e.g. rheumatoid arthritis Bauer S et al. Arthritis Res Ther 2006; 8; R171).

### SUMMARY

The present invention provides a novel class of oligopeptides that include amino acid sequences containing cleavage sites for fibroblast activation protein (FAP). These cleavage sites are derived from an FAP specific cleavage map of human collagen and from FAP cleavable peptides isolated from a random peptide library. These oligo-peptides are useful in assays that can determine the free FAP protease activity. Furthermore, the invention also provides a therapeutic prodrug composition, comprising a therapeutic drug linked to a peptide, which is specifically cleaved by FAP. The linkage substantially inhibits the non-specific toxicity of the drug, and cleavage of the peptide releases the drug, activating it or restoring its non-specific toxicity. Furthermore, the invention also provides a therapeutic protoxin composition, comprising a protein or peptide toxin in which a peptide sequence that is selectively cleaved by FAP is incorporated into the sequence of the protein/peptide. The incorporation of the peptide into the protein sequence inhibits non-specific toxicity of the toxin and cleavage of the peptide by FAP releases an inhibitory portion of the protein, leading to activation and restoration of the toxicity of the protein/peptide.

The invention also provides a method for treating cell proliferative disorders, including those involving the production of FAP, in subjects having or at risk of having such disorders. The method involves administering to the subject a therapeutically effective amount of the composition of the invention.

The invention also provides a method of producing the prodrug and protoxin composition of the invention. In another embodiment, the invention provides a method of detecting FAP activity in tissue. In yet another embodiment, the invention provides a method of selecting appropriate prodrugs and protoxins for use in treating cell proliferative disorders involving FAP-production.

In one aspect the invention features a peptide containing an amino acid sequence that includes a cleavage site specific for FAP or an enzyme having a proteolytic activity of FAP. The peptides of the invention are preferably not more than 20 amino acids in length, more preferably to more than ten amino acids in length, and even more preferably about 6 amino acids in length. The preferred amino acid sequences of the invention are linear. In an embodiment of the invention the amino acid sequence may be cyclical such that the cyclical form of the sequence is an inactive drug that can become an activated drug upon cleavage by FAP and linearization.

Provided herein, according to one aspect are peptides comprising an amino acid sequence having a cleavage site specific for an enzyme having a proteolytic activity of fibroblast activation protein (FAP), wherein the peptide comprises the sequence of any one of SEQ ID NO. 1-44 and peptide sequences listed in Tables 1, 2, and 3 and FIGS. 12 and 14.

In one embodiment, the peptides further comprise a nitrotyrosine quencher at the amino terminus of the peptide.

In one embodiment, the peptides further comprise a capping group attached to the N-terminus of the peptide, wherein the capping group inhibits endopeptidase activity on the peptide.

In another embodiment, the capping comprises one or more of acetyl, morpholinocarbonyl, benzyloxycarbonyl, glutaryl or succinyl substituents.

In one embodiment, the peptides further comprise an added substituent that renders the peptide water-soluble.

In one embodiment, the added substituent is a polymer. In a related embodiment, the polymer is selected from the group consisting of polylysine, polyethylene glycol (PEG), and a polysaccharide. In another related embodiment, the polysaccharide is selected from the group consisting of modified or unmodified dextran, cyclodextrin, and starch.

In one embodiment, the peptides further comprise one or more of an antibody or a peptide toxin attached to the amino terminus and/or the carboxy terminus of the peptide.

In one embodiment, the peptides further comprise a peptide toxin attached to the peptide.

In another embodiment, the peptide toxin comprise one or more of melittin, toxin cecropin B, bombolittin, magainin, samafotoxin, pardaxins, defensins and amphipathic synthetic toxins comprising combinations of the amino acids Lys (I), Leu (L) or Ala (A).

In another embodiment, the peptide is incorporated into the amino acid structure of a protein toxin such that hydrolysis of the peptide by FAP converts the toxin from an inactive to active state. Examples of such protein toxins include proaerolysin, produced by the bacteria Aeromonas hydrophilia, alpha toxin produced by Clostridium septicum, delta toxin produced by Bacillus thuringiensis, and n α-hemolysin produced by Staph aureus.

Provided herein, according to one aspect are peptide compositions comprising a plurality of peptides, each peptide comprising an amino acid sequence having a cleavage site specific for an enzyme having a proteolytic activity of FAP (FAP), wherein each peptide comprises (D/E)RG(E/A)(T/S)GPA or peptide sequences with Proline in P1 but having either nothing in P′1, Ala, Ser, Val in P′1, or Ala, Ser Val in P′1 and Gly in P′2.

Provided herein, according to one aspect polynucleotides encoding the peptides comprising the sequence of any one of SEQ ID NO. 1-44 and peptide sequences listed in Tables 1, 2, and 3 and FIGS. 12 and 14.

Provided herein, according to one aspect are compositions comprising a prodrug, the prodrug comprising a therapeutically active drug, and a peptide comprising an amino acid sequence having a cleavage site specific for an enzyme having a proteolytic activity FAP, wherein the peptide is 20 or fewer amino acids in length, and wherein the peptide is linked to the therapeutically active drug to inhibit the therapeutic activity of the drug, and wherein the therapeutically active drug is cleaved from the peptide upon proteolysis by an enzyme having a proteolytic activity of FAP.

In one embodiment, the peptide is linked directly to the therapeutic drug.

In another embodiment, the peptide is linked directly to a primary amine group on the drug.

In another embodiment, the peptide is linked to the therapeutic drug via a linker.

In a related embodiment, the linker is an amino acid sequence. In another related embodiment, the linker comprises a leucine residue.

In one embodiment, the therapeutically active drug is an anthracycline, a taxane, a vinca alkaloid, an antiandrogen, an antifolate, a nucleoside analog, a topoisomerase inhibitor, an alklating agent, a primary amine containing thapsigargins and thapsigargin derivatives or a targeted radiation sensitizer. In a related embodiment, the anthracycline is selected from the group consisting of doxorubicin, daunorubicin, epirubicin, and idarubicin. In another related embodiment, the taxane comprises one or more of paclitaxel or docetaxel. In yet another related embodiment, the vinca alkaloid comprises one or more of vincristine, vinblastine, or etoposide. In a related embodiment, the antiandrogen comprises one or more of biscalutamide, flutamide, nilutamide, or cyproterone acetate. In a related embodiment, the antifolate comprises methotrexate. In a related embodiment, the nucleoside analog comprises one or more of 5-Fluorouracil, gemcitabine, or 5-azacytidine. In another related embodiment, the topoisomerase inhibitor comprises one or more of Topotecan or irinotecan. In another related embodiment, the alkylating agent comprises one or more of cyclophosphamide, Cisplatinum, carboplatinum, or ifosfamide. In a related embodiment, the targeted radiation sensitizer comprises one or more of 5-fluorouracil, gemcitabine, topoisomerase inhibitors, or cisplatinum. In one embodiment, the therapeutically active drug inhibits a sarcoplasmic reticulum and endoplasmic reticulum Ca²⁺-ATPase (SERCA) pump. In one embodiment, the thapsigargin derivative is 8-O-(12-[L-leucinoylamino]dodecanoyl)-8-O-debutanoylthapsigargin (L12ADT).

In one embodiment, the therapeutically active drug has an LC₅₀ toward FAP-producing tissue of at most 20 μM. In a related embodiment, the therapeutically active drug has an LC₅₀ toward FAP-producing tissue of less than or equal to 2.0 μM.

Provided herein, according to one aspect are methods of producing a prodrug, the method comprising the step of linking a therapeutically active drug and a peptide comprising an amino acid sequence having a cleavage site specific for an enzyme having a proteolytic activity FAP, wherein the peptide is 20 or fewer amino acids in length, and wherein the peptide is linked to the therapeutically active drug to inhibit the therapeutic activity of the drug, and wherein the therapeutically active drug is cleaved from the peptide upon proteolysis by an enzyme having a proteolytic activity of FAP.

In one embodiment, the therapeutically active drug has a primary amine.

In another embodiment, the prodrug contains a linker between the peptide and the drag.

In one embodiment, the linker is an amino acid sequence comprising leucine.

In one embodiment, the peptides further comprise a capping group attached to the N-terminus of the peptide, the capping group inhibiting endopeptidase activity on the peptide.

In another embodiment, the capping group is selected from the group consisting of acetyl, morpholinocarbonyl, benzyloxycarbonyl, glutaryl, and succinyl substituents.

Provided herein, according to one aspect are methods of treating a FAP related disorder, comprising administering the compositions described herein in a therapeutically effective amount to a subject having the cell proliferative disorder.

In one embodiment, the disorder is benign. In a related embodiment, the disorder is malignant. In another related embodiment, the malignant disorder an epithelial cancer. In another related embodiment, the malignant disorder is one or more of epithelial cancers and inflammatory conditions (rheumatoid arthritis).

In one embodiment, the composition is administered as a single dose comprising at least about 7 mg/kg peptide. In a related embodiment, the composition is administered as a single dose comprising at least about 17.5 mg/kg peptide. In one embodiment, the composition is administered in doses of at least about 7 mg/kg peptide per day for at least 4 days.

Provided herein, according to one aspect are methods of detecting FAP-producing tissue comprising: contacting the tissue with a composition comprising a detectably labeled peptide of claim 1 for a period of time sufficient to allow cleavage of the peptide; and detecting the detectable label.

In one embodiment, the detectable label is a fluorescent label.

In another embodiment, the fluorescent label is selected from the group consisting of 7-amino-4-methyl coumarin, 7-amino-4-trifluoromethyl coumarin, rhodamine 110, and 6-aminoquinoline.

In one embodiment, the detectable label is a radioactive label.

In another embodiment, the radioactive label comprises one or more of tritium, carbon-14, or iodine-125.

In one embodiment, the detectable label is a chromophoric label. In a related embodiment, the detectable label is a chemiluminescent label.

Provided herein, according to one aspect are methods of selecting a fibroblast activation protein (FAP) activatable prodrug wherein the prodrug is substantially specific for target tissue comprising FAP-producing cells, comprising: a) contacting cells of a target tissue with a candidate prodrug composition with; b) contacting non-target tissue with the prodrug composition; and c) selecting a candidate prodrug composition that is substantially toxic towards target tissue cells, and not substantially toxic towards non-target tissue cells.

Provided herein, according to one aspect are methods of determining the activity of FAP in a comprising: a) contacting the sample with a composition comprising a detectably labeled peptide of any one of claim 1 for a period of time sufficient to allow cleavage of the peptide; b) detecting the detectable label; c) comparing a detection level with a standard.

Provided herein, according to one aspect are methods of imaging FAP-producing tissue, the method comprising: a) administering a peptide of claim 1 liked to a lipophilic imaging label to a subject having or suspected of having an FAP producing associated cell-proliferative disorder, b) allowing a sufficient period of time to pass to allow cleavage of the peptide by FAP and to allow clearance of uncleaved peptide from the subject to provide a reliable imaging of the imaging label; and c) imaging the subject.

Provided herein, according to one aspect are methods of identifying a FAP substate comprising a) incubating a random peptide library with FAP; b) detecting a peptide cleaved by FAP; and c) determining the sequence of the cleaved peptide, wherein the peptides comprise a label which is detectable only after cleavage by FAP.

Provided herein, according to one aspect are recombinant polynuclietides encoding the sequence of any one of SEQ ID NO. 1-44 and peptide sequences listed in Tables 1, 2, and 3 and FIGS. 12 and 14.

Provided herein, according to one aspect are cells transformed with a recombinant polynucleotide encoding the sequence of any one of SEQ ED NO. 1-44 and peptide sequences listed in Tables 1, 2, and 3 and FIGS. 12 and 14.

Provided herein, according to one aspect are transgenic organisms comprising a recombinant encoding the sequence of any one of SEQ ID NO. 1-44 and peptide sequences listed in Tables 1, 2, and 3 and FIGS. 12 and 14.

Provided herein, according to one aspect are methods method of producing a polypeptide of any one of SEQ ID NO. 1-44 and peptide sequences listed in Tables 1, 2, and 3 and FIGS. 12 and 14, the method comprising: a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and b) recovering the polypeptide. Other embodiments of the invention are disclosed infra.

### BRIEF DESCRIPTION OF THE DRAWINGS

FIG. 1(a) shows the digestion of quenched gelatin and (b) shows digestion of quenched Collagen I.

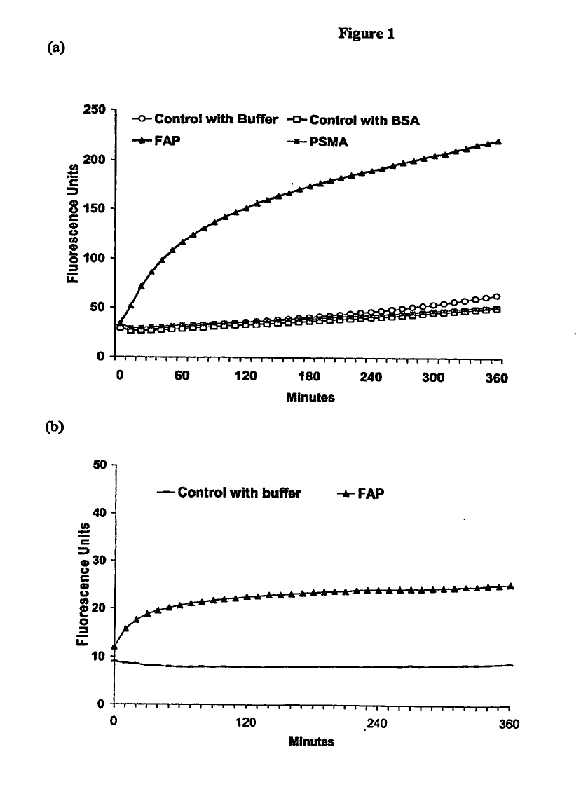

FIG. 2 shows MALDI spectra for FAP digestion of human collagen I and positive and negative controls.

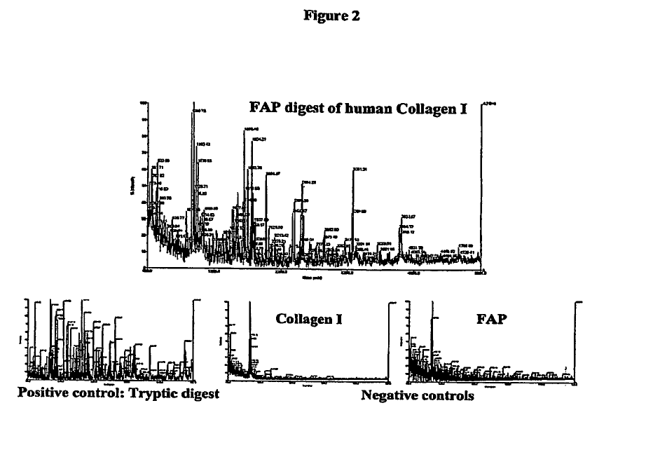

FIG. 3 depicts FAP digestion of recombinant human gelatin of size 100 Kda.

FIG. 4 depicts base peak chromatogram for FAP digest of 100 KDa Gelatin.

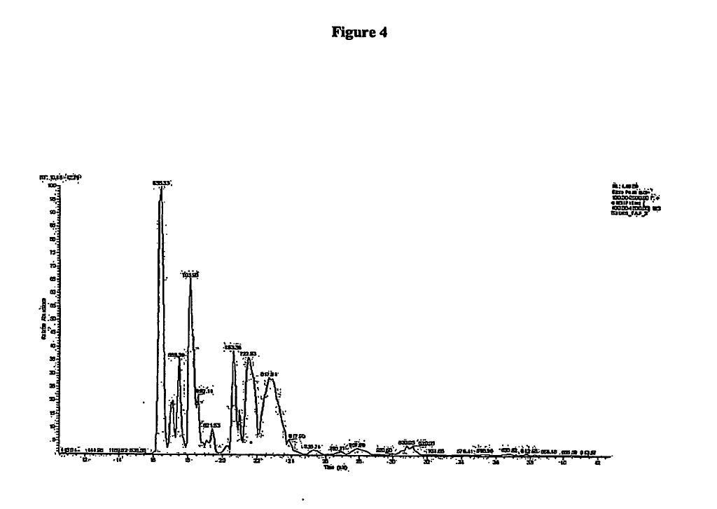

FIG. 5 depicts Collision Induced Decay (CID) sample spectra for AGKDGEAGAQGPPGP.

FIG. 6 shows antitumor effect of the FAP activated prodrug [consisting of the FAP selective peptide sequence Mu-SGEAGPA (where Mu is morpholinocarbonyl protecting group) coupled to 12 ADT (12-aminododecanoyl thapsigargin) a potent cytotoxic analog of the natural product thapsigargin] against human MDA-MB231 breast cancer xenografts growing in nude mice.

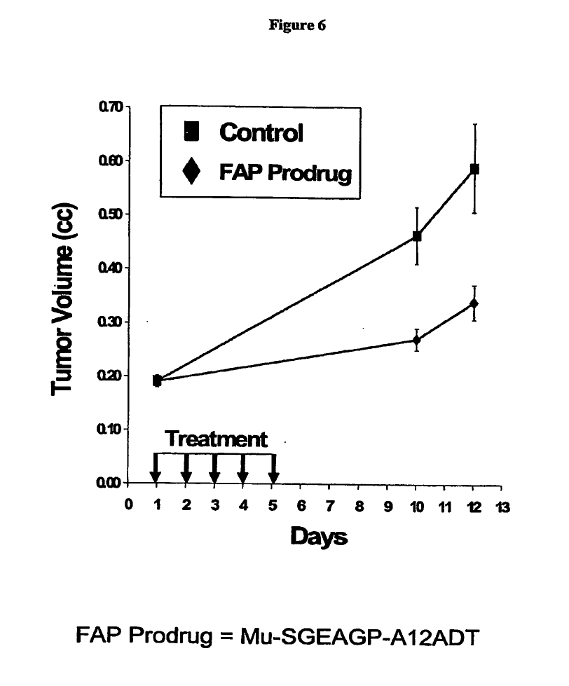

FIG. 7 shows Levels of FAP prodrug and free A-12ADT in MDA-MB231 tumor tissue and plasma following five daily intravenous injections of 7 mg/kg prodrug.

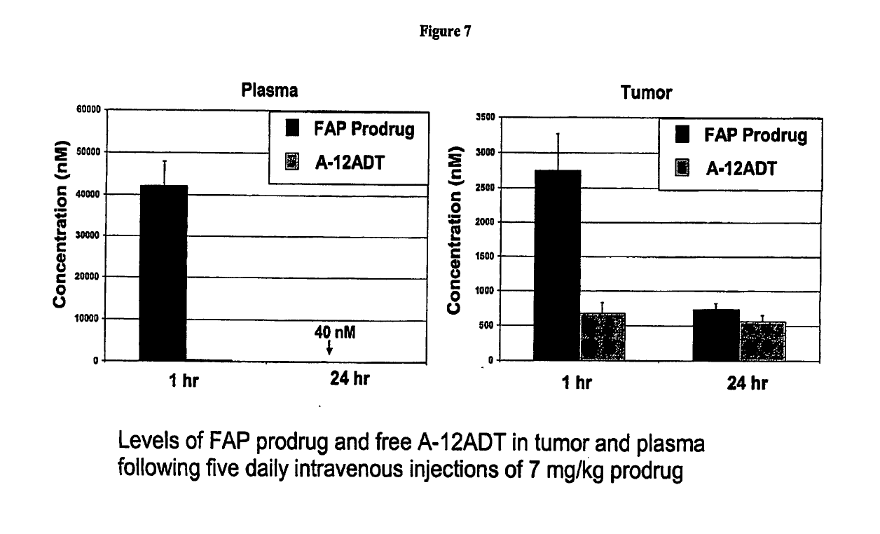

FIG. 8 depicts FAP expression in (A) Stroma from series of epithelial and non-epithelial cancers; (B) Stroma from breast cancer samples compared to breast cancer epithelial cells.

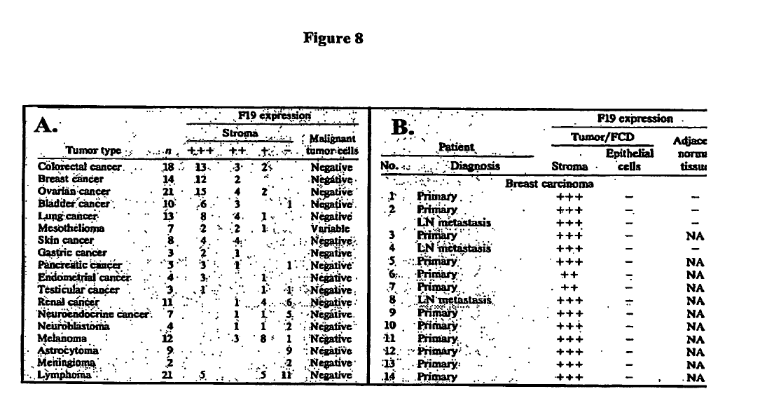

FIG. 9 depicts a model of TG analog containing long hydrophobic side chain coupled to amino acid showing hydrophobic side chain in channel and amino acid interacting with the cytoplasm outside of the channel.

FIG. 10 depicts the chemical structure of thapsigargin analog modified in O-8 position with 12-aminododecanoyl side chain coupled to carboxyl-group of an amino acid.

FIG. 11 depicts fluorescence quenched Collagen I labeled with the fluorophore FITC was incubated with purified FAP or Trypsin as positive control. Protein hydrolysis releases FITC labeled peptide fragments resulting in increased fluorescence intensity over time. Inset shows Western blot analysis demonstrating single band of His-tagged FAP after Ni-resin purification

FIG. 12 depicts the complete map of FAP cleavage sites within an 8.5 kDa fragment of recombinant human gelatin prepared from human collagen I.

FIG. 13 depicts (A) FAP Hydrolysis rates of fluorescently quenched peptide with indicated peptide sequences assayed at concentration of 30 μM. Relative change in fluorescence measured in 96 well fluorescent plate reader (Fluoroscan II). (B) Michaelis Menten plots of PGP//AGQ and VGP//AGK with kinetic parameters calculated using Enzyme Kinetics Module from Sigma Plot 8.0 software.

FIG. 14 depicts the complete map of FAP cleavage sites within 100 kDa recombinant human gelatin prepared from human collagen I.

FIG. 15 depicts the positional analysis of amino acids from FAP cleavage sites within 100 kDa recombinant human gelatin. (Blue column represents percent of each amino acid in positions P7-P′1 for all cleavage sites; Purple column indicates percent of each amino acid in positions P7-P′1 in only those sequences having Proline at cleavage site in the P1 position.

FIG. 16 shows hydrolysis by purified FAP of peptide substrates derived from the 100 kDa gelatin cleavage map at various concentrations.

FIG. 17 shows the flow cytometric traces of individual FAP-transfected and empty vector transfected controls demonstrating positive expression of FAP in both cell lines.

FIG. 18 shows the hydrolysis of fluorescently quenched FAP peptide substrates in conditioned media from FAP-transfected MDA-MB-231 cells and control cells transfected with PSMA.

### DETAILED DESCRIPTION

The present invention is based, in part, on a highly consistent trait of tumor stromal fibroblasts is the induction of fibroblast-activation protein-alpha (FAP). FAP was demonstrated to be a membrane bound serine protease that has both prolyl dipeptidase as well as gelatinase and collagenase activity (reviewed in 17). FAP was also demonstrated to be selectively expressed by reactive stromal fibroblasts in >90% of epithelial cancers studied with little to no expression in normal or cancerous epithelial cells or normal stromal fibroblasts (16). Reactive stromal expression of FAP, therefore, represents a target for selective activation of prodrugs within the tumor microenvironment.

As used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with various embodiments of the invention. Nothing herein is to be construed as an admission that the invention is not entitled to antedate such disclosure by virtue of prior invention.

As used herein the term “fibroblast-activation protein-alpha” (FAP) refers to fibroblast-activation protein-alpha as well as other proteases that have the same or substantially the same proteolytic cleavage specificity as FAP. As used herein, the term “naturally occurring amino acid side chain” refers to the side chains of amino acids known in the art as occurring in proteins, including those produced by post-translational modifications of amino acid side chains.

The term “contacting” refers to exposing tissue to the peptides, therapeutic drugs or prodrugs of the invention so that they can effectively inhibit cellular processes, or kill cells. Contacting may be in vitro, for example by adding the peptide, drug or prodrug to a tissue culture to test for susceptibility of the tissue to the peptide, drug or prodrug. Contacting may be in vivo, for example administering the peptide, drug, or prodrug to a subject with a cell or in vitro

By “peptide” or “polypeptide” is meant any chain of amino acids, regardless of length or post-translational modification (e.g., glycosylation or phosphoxylation). As written herein, amino acid sequences are presented according to the standard convention, namely that the amino-terminus of the peptide is on the left, and the carboxy terminus on the right.

The term “antibody” refers to intact immunoglobulin molecules as well as to fragments thereof, such as Fab, F(ab′)₂, and Fv fragments, which are capable of binding an epitopic determinant. Antibodies that bind FAP polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “aptamer” refers to a nucleic acid or oligonucleotide molecule that binds to a specific molecular target. Aptamers may be derived from an in vitro evolutionary process (e.g., SELEX (Systematic Evolution of Ligands by EXponential Enrichment), described in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules.

The term “antisense” refers to any composition capable of base-pairing with the “sense” (coding) strand of a polynucleotide having a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic FAP, or of any oligopeptide thereof, to induce a specific immune response in appropriate animals or cells and to bind with specific antibodies.

A “composition comprising a given polynucleotide” and a “composition comprising a given polypeptide” can refer to any composition containing the given polynucleotide or polypeptide. The composition may comprise a dry formulation or an aqueous solution. Compositions comprising polynucleotides encoding FAP or fragments of FAP may be employed as hybridization probes. “Conservative amino acid substitutions” are those substitutions that are predicted to least interfere with the properties of the original protein, e.g., the structure and especially the function of the protein is conserved and not significantly changed by such substitutions. The table below shows amino acids which may be substituted for an original amino acid in a protein and which are regarded as conservative amino acid substitutions Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “detectable label” refers to a reporter molecule or enzyme that is capable of generating a measurable signal and is covalently or noncovalently joined to a polynucleotide or polypeptide.

The terms “percent identity” and “% identity,” as applied to polynucleotide sequences, refer to the percentage of identical nucleotide matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity between polynucleotide sequences may be determined using one or more computer algorithms or programs known in the art or described herein. For example, percent identity can be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989; CABIOS 5:151-153) and in Higgins, D. G. et al. (1992; CABIOS 8:189-191). For pairwise alignments of polynucleotide sequences, the default parameters are set as follows: Ktuple=2, gap penalty=5, window=4, and “diagonals saved”=4. The “weighted” residue weight table is selected as the default.

Alternatively, a suite of commonly used and freely available sequence comparison algorithms which can be used is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nagov/gorf/b12.html. The “BLAST 2 Sequences” tool can be used for both blasts and blastp (discussed below). BLAST programs are commonly used with gap and other parameters set to default settings. For example, to compare two nucleotide sequences, one may use blastn with the “BLAST 2 Sequences” tool Version 2.0.12 (Apr. 21, 2000) set at default parameters. Such default parameters may be, for example:
Matrix: BLOSUM62
Reward for match: 1
Penalty for mismatch: −2
Open Gap: 5 and Extension Gap: 2 penalties
Gap x drop-off: 50
Expect: 10
Word Size: 11
Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured. Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of identical residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. The phrases “percent similarity” and “% similarity,” as applied to polypeptide sequences, refer to the percentage of residue matches, including identical residue matches and conservative substitutions, between at least two polypeptide sequences aligned using a standardized algorithm. In contrast, conservative substitutions are not included in the calculation of percent identity between polypeptide sequences.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, windows=5, and “diagonals saved”=5. The PAM250 matrix is selected as the default residue weight table.

Alternatively the NCBI BLAST software suite may be used. For example, for a pairwise comparison of two polypeptide sequences, one may use the “BLAST 2 Sequences” tool Version 2.0.12 (Apr. 21, 2000) with blastp set at default parameters. Such default parameters may be, for example:
Matrix: BLOSUM62
Open Gap: 11 and Extension Gap: 1 penalties
Gap x drop-off: 50
Expect 10
Word Size: 3
Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The term “modulate” refers to a change in the activity of FAP. For example, modulation may cause an increase or a decrease in protein activity, binding characteristics, or any other biological, functional, or immunological properties of FAP.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense stand, to peptide nucleic acid (PNA), or to any DNA-like or RNA-like material.

The term “substantially purified” refers to nucleic acid or amino acid sequences that are removed from their natural environment and are isolated or separated, and are at least about 60% free, preferably at least about 75% free, and most preferably at least about 90% flee from other components with which they are naturally associated.

A “substitution” refers to the replacement of one or more amino acid residues or nucleotides by different amino acid residues or nucleotides, respectively.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. A “variant” of a particular nucleic acid sequence is defined as a nucleic acid sequence having at least 40% sequence identity to the particular nucleic acid sequence over a certain length of one of the nucleic acid sequences using blasts with the “BLAST 2 Sequences” tool Version 2.0.9 (May 7, 1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing during mRNA processing. The corresponding polypeptide may possess additional functional domains or lack domains that awe present in the reference molecule. Species variants are polynucleotides that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity or sequence similarity to the particular polypeptide sequence over a certain length of one of the polypeptide sequences using blastp with the “BLAST 2 Sequences” tool Version 2.0.9 (May 7, 1999) set at default parameters. Such a pair of polypeptides may show, for example, at least 50%, at least 60%, at least 70%, at least 80%, at least 85%, at least 90%, at least 91%, at least 92%, at least 93%, at least 94%, at least 95%, at least 96%, at least97%, at least 98%, or at least 99% or greater sequence identity or sequence similarity over a certain defined length of one of the polypeptides.

The terms “Seat” or “treatment” refer to both therapeutic treatment and prophylactic or preventative measures, wherein the object is to prevent or slow down (lessen) an undesired physiological change or disorder, such as the development or spread of cancer. For purposes of this invention, beneficial or desired clinical results include, but are not limited to, alleviation of symptoms, diminishment of extent of disease, stabilized (e.g., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented. The terms “treating”, “treat”, or “treatment” embrace both preventative, e.g., prophylactic, and palliative treatment.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (e.g., slow to some extent and preferably stop) cancer cell infiltration into peripheral organs; inhibit (e.g., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can, for example, be measured by assessing the time to disease progression (TTP) and/or determining the response rate (RR).

The term “bioavailability” refers to the systemic availability (e.g., blood/plasma levels) of a given amount of drug administered to a patient. Bioavailability is an absolute term that indicates measurement of both the time (rate) and total amount (extent) of drug that reaches the general circulation from an administered dosage form.

An “FAP related disorder,” as used herein includes disorders wherein FAP is expressed, e.g., epithelial cancer and other disorders described infra.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include epithelial cancers and other cancers described infra.

The term “prodrug” as used in this application refers to a precursor or derivative form of a pharmaceutically active substance that is less cytotoxic to tumor cells compared to the parent drug and is capable of being enzymatically or hydrolytically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, .beta.-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described herein.

The term “protoxin” are sued herein refer, for example, to peptide toxins linked to the FAP substrate peptides of the invention. Protein toxins, include, for example, the 26 amino acid toxin melittin and the 35 amino acid toxin cecropin B. Both of these peptide toxins have shown toxicity against cancer cell lines. The N-terminal amino acid of the peptide may also be attached to the C-terminal amino acid either via an amide bond formed by the N-terminal amine and the C-terminal carboxyl, or via coupling of side chains on the N-terminal and C-terminal amino acids or via disulfide bond formed when the N-terminal and C-terminal amino acids both consist of the amino acid cysteine. Further, it is envisioned that the peptides described herein can be coupled, via the carboxy terminus, to a variety of peptide toxins (for example, melittin and cecropin are examples of insect toxins. Other examples include, for example, toxin cecropin B, bombolittin, magainin, sarafotoxin, pardaxins, defensins and amphipathic synthetic toxins comprising combinations of the amino acids Lys (K), Leu (L) or Ala (A).

Peptide toxins are incorporated into the amino acid structure of a protein toxin such that hydrolysis of the peptide by FAP converts the toxin from an inactive to active state. Examples of such protein toxins include proaerolysin, produced by the bacteria Aeromonas hydrophilia, alpha toxin produced by Clostridium septicum, delta toxin produced by Bacillus thuringiensis, and n α-chemolysin produced by Staph aureus.

A “liposome” is a small vesicle composed of various types of lipids, phospholipids and/or surfactant that is useful for delivery of a drug (such as the compositions disclosed herein and, optionally, a chemotherapeutic agent) to a mammal. The components of the liposome are commonly arranged in a bilayer formation, similar to the lipid arrangement of biological membranes.

The term “package insert” is used to refer to instructions customarily included in commercial packages of therapeutic products, that contain information about the indications, usage, dosage, administration, contraindications and/or warnings concerning the use of such therapeutic products.

The phrase “pharmaceutically acceptable salt,” as used herein, refers to pharmaceutically acceptable organic or inorganic salts of a compound of the invention. Exemplary salts include, but are not limited, to sulfate, citrate, acetate, trifluoroacetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascotbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (e.g., 1,1′-methylene-bis-(2-hydroxy-3-naphthoate)) salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ions. A “solvate” refers to an association or complex of one or more solvent molecules and a compound of the invention. Examples of solvents that form solvates include, but are not limited to, water, isopropanol, ethanol, methanol, DMSO, ethyl acetate, acetic acid, and ethanolamine. The term “hydrate” refers to the complex where the solvent molecule is water.

The term “protecting group” or “Pg” refers to a substituent that is commonly employed to block or protect a particular functionality while reacting other functional groups on the compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBz) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include —CH.sub.2CH.sub.2SO.sub.2Ph, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see T. W. Greene, Protective Groups in Organic Synthesis, John Wiley & Sons, New York, 1991.

The term “animal” refers to humans (male or female), companion animals (e.g., dogs, cats and horses), food-source animals, zoo animals, marine animals, birds and other similar animal species. “Edible animals” refers to food-source gals such as cows, pigs, sheep and poultry.

The phrase “pharmaceutically acceptable” indicates that the substance or composition must be compatible chemically and/or toxicologically, with the other ingredients comprising a formulation, and/or the mammal being treated therewith.

The term “non-naturally occurring amino acid” refers to amino acids that are not normally found in living organisms.

The terms “isolated” and the term “purified” in the context of “isolated and purified peptide sequences” refer to the separation of the desired peptide sequence(s) from non-desired peptide sequences and other contaminants (e.g. lipids, carbohydrates, nuclei acids, etc.). The terms “isolated” and “purified” do not necessarily mean isolated and purified to 100% homogeneity, although this is also contemplated. Rather, the terms mean isolated and purified to at least 50% homogeneity. In a preferred embodiment, the peptide sequences are isolated and purified to at lest 75% homogeneity. In a more preferred embodiment, the peptide sequences are isolated and purified to at least 90% homogeneity. After isolation and purification, the peptide sequences can then be mixed with or added to other compounds or molecules.

The term “at least one symptom is reduced” means that, after treatment at least one of any number of symptoms is reduced. The reduction need not be complete. That is, a partial reduction in the symptom is contemplated. Additionally, the symptom need not be reduced permanently. A temporary reduction in at least one symptom is contemplated by the present invention.

The term “subject at risk for cancer” is a person or patient having an increased chance of cancer (relative to the general population). Such subjects may, for example, be from families with a history of cancer. Additionally, subjects at risk may be individuals in which there is a genetic history of a particular cancer associated with race, nationality or heritage or exposure to an environmental trigger.

### FAP

FAP is a member of the enzyme class known as post-prolyl peptidases that are uniquely capable of cleaving the Pro-X amino acid bond. These enzymes have been demonstrated to play a role in cancer biology and are capable of modifying bioactive peptides (17). This group of proteases includes the well characterized dipeptidyl peptidase IV (DPPIV) and FAP as well as DPP6, DPP7, DPP8, DPP9, DPP10, prolyl carboxypeptidase, and prolyl oligopeptidase, table 1 (17). The substrate preferences for many of these prolyl peptidases are not entirely known but, like DPPIV most have dipeptidase functionality (DPP6 and DPP10 are inactive due to an amino acid substitution in the catalytic triad). FAP is highly homologous to DPPIV (17). Of the known prolyl peptidases only DPPIV and FAP are integral membrane proteins (18). However, FAP differs from DPPIV in that it also has gelatinase and collagenase activity (17,19). This additional gelatinase/collagenase activity may be unique to FAP among the family of prolyl proteases. Unlike DPPIV, FAP is also not widely expressed in most normal tissues (17).

FAP was originally reported to be a cell-surface antigen recognized by the F19 monoclonal antibody (MAb) on human astrocytes and sarcoma cell lines in vitro (20). In one series using frozen sections of human tissues, FAP was detected in the stroma of over 90% of malignant breast, colorectal, skin and pancreatic tumors (16,21), Table 2 (Appendix B). In a small study, FAP was also detected in the stroma of 7/7 prostate cancers (22). FAP is also expressed by a proportion of soft tissue and bone sarcomas (16). FAP positive fibroblasts also accompany newly formed tumor blood vessels (21). FAP is also expressed in reactive fibroblasts in healing wounds, rheumatoid arthritis, liver cirrhosis and in some fetal mesenchymal tissues (16). In contrast, most normal adult tissues demonstrate no detectable FAP protein expression (16).

Studies to date suggest that FAP's role in tumor growth may be highly contextual and in some cases, FAP expression may itself be growth inhibitory to tumors. Unlike these inhibitory strategies, the prodrug strategy described here takes advantage of FAP's enzymatic activity to selectively activate a highly potent cytotoxin in the peritumoral fluid. Because the TG analog is highly lipophilic, release from the water soluble peptide leads to accumulation of the toxin in the tumor tissue over time. Such activation and drug accumulation will lead to death of tumor stromal cells, but will also generate a significant bystander effect leading to death of tumor cells and endothelial cells within the stromal compartment.

It was demonstrated that FAP has both dipeptidyl peptidase and collagenolytic activity capable of degrading gelatin and type I collagen. The expression and enzyme activity of FAP in benign and malignant melanocytic skin tumors has been established, indicating a possible role for FAP in the control of tumor cell growth and proliferation during melanoma carcinogenesis (Huber et al (2003) Jour. of Investigative Dermatology 120(2):182-188), colorectal cancer (Satoshi et al (2003) Cancer letters 199(1):91-98), and breast cancer (Goodman et al (2003) Clinical & Exp. Metastasis 20(5):459-470), as well as all of breast, colon, and lung cancer (Park et al (1999) J. Biol. Chem. 274:36505-36512). Furthermore, FAP seems to upregulated in cirrhosis (Levi, M T et al (1999) Hepatology 29:1768-1778), fibromatosis (Skabitz, K M et al J. Clin. Lab. Med. (2004) 143(2):89-98), and rheumatoid arthritis.

Maturation of blood cells via hematopoiesis involves cytokines and their regulation by the seine proteases CD26/dipeptidyl-peptidase IV (DPP-IV), as well as FAP (McIntyre et al (2004) Drugs of the Future 29(9):882-886; Ajamni et al (2003) Biochemistry 42(3):694-701). The human fibroblast activation protein (FAPα) is a M.sub.f 95,000 cell surface molecule originally identified with monoclonal antibody (mAb) F19 (Rettig et al. (1988) Proc. Natl. Acad. Sci. USA 85, 3110-3114; Rettig et al. (1993) Cancer Res. 53, 3327-3335; Rettig et al (1994) Intl. Jour. of Cancer 58(3):385-392). The FAP gene, localized to chromosome 2 in humans (Mathew et al (1995) Genomics 25(1):335-337) is a 2812 nt sequence with an open reading frame of 2277 bp conserved throughout a variety of species including mouse, hamster, and Xenopus laevis (Scanlan et al (1994) Proc. Natl. Acad. Sci. USA 91:5657-5661; Park et al (1999) J. Biol. Chem. 274:36505-36512; Niedermeyer et al (1998) Eur. J. Biochem. 254:650-654). The corresponding FAP protein product contains 759 or 760 amino acids and has a calculated molecular weight of about 88 kDa. The primary amino acid sequence is homologous to type II integral membrane proteins, which are characterized by a carboxy-terminal end that is large and corresponds to the extra-cellular domain (ECD), a hydrophobic transmembrane segment, and a short cytoplasmic tail. FAP is highly homologous to dipeptidyl peptidase IV (DDPIV) in various species, with 61% nucleotide sequence identity and 48% amino acid sequence identity to DPPIV. Although both FAP and DDPIV have peptidase (protease) activity, biochemical and serological studies show that these proteins are significantly different in their enzymatic activity with synthetic substrates as well as their functional activation of T lymphocytes (DDPIV induction) or reactive stromal fibroblasts (FAP induction (Mathew et al (1995) Genomics w5:335-337). The FAPα cDNA codes for a type II integral membrane protein with a large extracellular domain, trans-membrane segment, and short cytoplasmic tail (Scanlan et al. (1994) Proc. Natl. Acad. Sci. USA 91, 5657-5661; U.S. Pat. No. 6,846,910; WO 97/34927; U.S. Pat. No. 5,767,242; U.S. Pat. No. 5,587,299; U.S. Pat. No. 5,965,373). FAPα shows 48% anino acid sequence identity to the T-ell activation antigen CD26, also known as dipeptidyl peptidase IV (DPPIV; EC 3.4.14.5), a membrane-bound protein with dipeptidyl peptidase activity. FAP.alpha. has enzymatic activity and is a member of the serine protease family, with serine 624 being critical for enzymatic function WO 97/34927; U.S. Pat. No. 5,965,373). FAPα is selectively expressed in reactive stromal fibroblasts of many histological types of human epithelial cancers, granulation tissue of healing wounds, and malignant cells of certain bone and soft tissue sarcomas. Normal adult tissues are generally devoid of detectable FAP.alpha.: (Chen et al (2003) Adv. Exp. Med. Biol. 524:197-203), but some fetal mesenchymal tissues transiently express the molecule. In contrast, most of the common types of epithelial cancers, including >90% of breast, non-small-cell lung, and colorectal carcinomas, contain FAPα-reactive stromal fibroblasts. These FAPα⁺ fibroblasts accompany newly formed tumor blood vessels, forming a distinct cellular compartment interposed between the tumor capillary endothelium and the basal aspect of malignant epithelial cell clusters (Welt et al. (1994) J. Clin. Oncol. 12(6), 1193-1203). While FAPα⁺ stromal fibroblasts are found in both primary and metastatic carcinomas, the benign and premalignant epithelial lesions tested, such as fibroadenomas of the breast and colorectal adenomas, only rarely contain FAPα⁺ stromal cells. Based on the restricted distribution pattern of FAPα in normal tissues and its uniform expression in the supporting stroma of many malignant tumors, the disclosed prodrugs were designed to exploit the expression of FAP for clinical efficacy.

The treatment of epithelial carcinomas including breast, lung, colorectal, head and neck, pancreatic, ovarian, bladder, gastric, skin, endometrial, ovarian, testicular, esophageal, prostatic and renal origin; 2) Bone and soft-tissue sarcomas: Osteosarcoma, chondrosarcoma, fibrosarcoma, malignant fibrous histiocytoma (MFH), leiomyosarcoma; 3) Hematopoietic malignancies: Hodgkin's and non-Hodgkin's lymphomas; 4) Neuroectodermal tumors: Peripheral nerve tumors, astrocytomas, melanomas; 5) Mesotheliomas.

A high-resolution X-ray crystal structure of the extracellular domain of FAPα revealed a difference from DPP-IV in their active sites. Kinetic analysis of an active site mutant of FAPα, A657D, with dipeptide substrates showed an increase in the rate of cleavage for a free amino terminus substrate but a decrease for the corresponding N-benzyloxycarbonyl substrate, relative to wild type FAPα (Aertgeerts et al (2005) J. Biol. Chem., April; 10. 1074/jbc.C500092200).

### Peptides and Substrates

In one embodiment, the agents that substrates of FAP comprise a peptide that comprises the sequence VGPAGK [SEQ ID NO.: 1]; GARGQA [SEQ ID NO.: 2]; PPGPPGPA [SEQ ID NO.: 3]; (D/E)RG(E/A)(T/S)GPA [SEQ ID NO: 4]; DRGETGPA [SEQ ID NO: 5]; RTGDAGPA [SEQ ID NO: 6); ASGPAGPA [SEQ ID NO: 7]; DRGETGPA (SEQ ID NO: 8]; DKGESGPA [SEQ ID NO: 9]; AKGEAGPA [SEQ ID NO: 10]; PPGPPGPA [SEQ ID NO: 11]; EPGPPGPA [SEQ ID NO: 12]; DAGPPGPA [SEQ ID NO: 13]; GETGPAGA [SEQ ID NO: 14]; QPSGPAGA [SEQ ID NO: 15]; ERGETGPA [SEQ ID NO: 16]; DRGATGPA [SEQ ID NO: 17]; DRGESGPA [SEQ ID NO: 18]; DPGETGPA [SEQ ID NO: 19]; LNGLPGA [SEQ ID NO: 20]; PSGPAGPA [SEQ ID NO: 21]; PAGAAGPA [SEQ ID NO: 22]; FPGARGPA [SEQ ID NO: 23]; FQGLPGPA [SEQ ID NO: 24]; PLGAPGPA [SEQ ID NO: 25]; PPGAVGPA [SEQ ID NO: 26]; MGFPGPA [SEQ ID NO: 27]; RVGPPGPA [SEQ ID NO: 28]; AGPVGPPA [SEQ ID NO: 29]; AGPPGPPA [SEQ ID NO: 30]; EPGASGPA [SEQ ID NO: 31]; ETGPAGPA [SEQ ID NO: 32]; PPGAVGPA [SEQ ID NO: 33]; AQGPPGPA [SEQ ID NO: 34]; KTGPPGPA [SEQ ID NO: 35]; VMGFPGPA [SEQ ID NO: 36]; SGEAGPA [SEQ ID NO: 37] and portions and variants thereof.

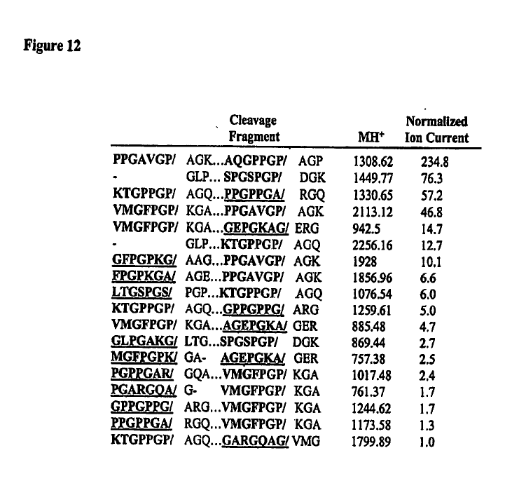

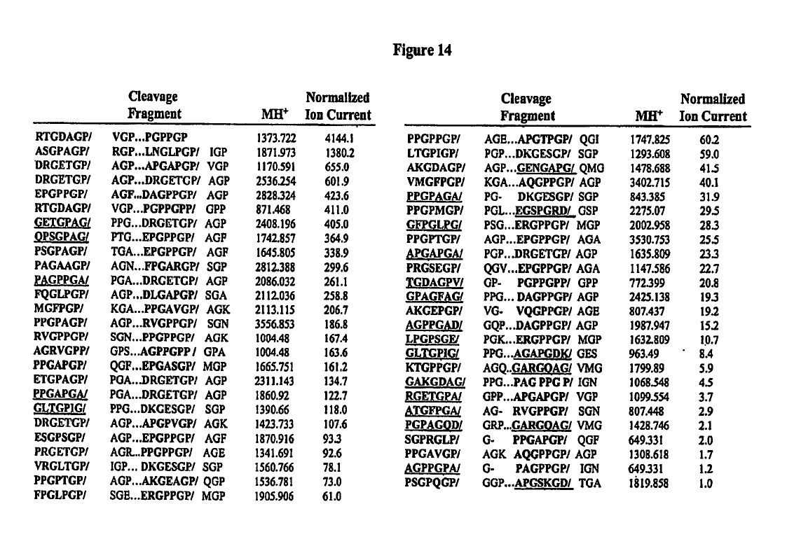

In other embodiments, substrates of FAP comprise a peptide that comprises the sequence of a peptides in the cleavage maps in Tables 1, 2 and 3 and FIGS. 12 and 14. In another embodiment, the substrates of FAP comprise a peptide that comprises the sequence XXXXX-A [SEQ ID NO: 38]; XXXX-AG [SEQ ID NO: 39]; XXXX-AGG [SEQ ID NO: 40]; XXXX-S [SEQ ID NO: 41]; XXXX-SG [SEQ ID NO: 42]; XXXX-V [SEQ ID NO: 43]; XXXXVG [SEQ ID NO: 44], wherein X is any amino acid, and portions and variants thereof.

Other peptide substrates of FAP falling within the scope of the invention include peptides with Prolines are most cleavage was found after Pro. Other peptides may contain the following amino acids as FAP was found to cleave after: Ala (e.g. A/A, A/G, A/P, A/R), Asp (e.g., D/G, D/T), Gly (e.g., G/A, G/E, G/L, G/Q, G/P, G/V), Glu (e.g., E/P), Lys (e.g., K/A, K/G), Ser (e.g., S/P) and Val (e.g., V/G).

Other embodiments include FAP substrate peptides with varying lengths in the P′ positions (e.g., P′1-P′3). That is, sequences with Proline in P1 but having either nothing in P′1, Ala, Ser, Val in P′1, or Ala, Ser Val in P′1 and Gly in P′2.

Other peptides would have the following sequences for FAP showed a preference for Asp or Glu, Arg or Ala residues in A7, Arg or Lys in P6, Ala, Asp or Glu in P4, Ala, Ser or Thr in P3 and Ala, Ser or Val in P′1 and Gly in P′2.

The peptides of the present invention may be synthesized by methods known in the art. For example, peptides may be synthesized by the methods of U.S. Pat. Nos. 6,632,922; 6,649,136; 6,310,180; 4,749,742. Peptides may also be synthesized on automated peptide synthesizing machines (e.g., the Symphony/Multiplex.™. automated peptide synthesizer (Protein Technologies, Inc, Tucson, Ariz.) or the Perkin-Elmer (Applied Biosystems, Foster City, Calif.) Model 433A automated peptide synthesizer).

Further, deletion of one or more amino acids can also result in a modification of the structure of the resultant molecule without significantly altering its biological activity. This can lead to the development of a smaller active molecule without significantly altering its biological activity. This can lead to the development of a smaller active molecule that would also have utility. For example, amino or carboxy-terminal amino acids which may not be required for biological activity of the particular peptide can be removed. Peptides of the invention include any analog, homolog, mutant or isomer or derivative of the peptides disclosed in the present invention, as long as bioactivity described herein remains. The peptides described in one embodiment have sequences comprised of L-amino acids; however, D-forms of the amino acids can be synthetically produced and used in the peptides described herein. In yet another embodiment, the amino acids are non-naturally occurring amino acids, which are known to one of skill in the art

The peptides of the invention include peptides that are conservative variations of those peptides specifically exemplified herein. The term “conservative variation” as used herein denotes the replacement of an amino acid residue by another, biologically similar residue. Examples of conserved variations include the substitution of one hydrophobic residue such as isoleucine, valine, leucine, alanine, cysteine, glycine, phenylalanine, proline, tryptophan, tyrosine, norleucine or methionine for another or the substitution of one polar residue for another such as the substitution of arginine for lysine or histidine, glutamic for aspartic acids or glutamine for asparagine, and the like. Neutral hydrophilic amino acids that can be substituted for one another include asparagine, glutamine, serine, and threonine. Such conservative substitutions are within the definitions of the classes of peptides of the invention. The peptides that are produced by such conservative variation can be screened for suitability of use in the prodrugs of the invention according to the methods for selecting prodrugs provided herein.

A wide variety of groups can be linked to the carboxy terminus of the peptides. Notably, therapeutic drugs can be linked to this position. In this way advantage is taken of the FAP specificity of the cleavage site, as well as other functional characteristics of the peptides of the invention. Preferably, the therapeutic drugs are linked to the carboxy terminus of the peptides, either directly or through a linker group. The direct linkage is preferably through an amide bond, in order to utilize the proteolytic activity and specificity of FAP. If the connection between the therapeutic drug and the amino acid sequence is made through a linker, this connection is also preferably made through an amide bond, for the same reason. This linker may be connected to the therapeutic drug through any of the bond types and chemical groups known to those skilled in the art. The linker may remain on the therapeutic drug, or may be removed soon thereafter, either by further reactions or in a self-cleaving step. Self-cleaving linkers are those linkers that can intramolecularly cyclize and release the drug or undergo spontaneous S_{N1} solvolysis and release the drug upon peptide cleavage.

Other materials such as detectable labels or imaging compounds can be linked to the peptide. Groups can be linked to the amino terminus of the peptides, including such moieties as antibodies, and peptide toxins, including the 26 amino acid toxin melittin and the 35 amino acid toxin cecropin B for example. Both of these peptide toxins have shown toxicity against cancer cell lines. The N-terminal amino acid of the peptide may also be attached to the C-terminal amino acid either via an amide bond formed by the N-terminal amine and the C-terminal carboxyl, or via coupling of side chains on the N-terminal and C-terminal amino acids or via disulfide bond formed when the N-terminal and C-terminal amino acids both consist of the amino acid cysteine. Further, it is envisioned that the peptides described herein can be coupled, via the carboxy terminus, to a variety of peptide toxins (for example, melittin and cecropin are examples of insect toxins), so that cleavage by FAP liberates an active toxin. Additionally, the peptide could be coupled to a protein such that the protein is connected at the carboxy terminal amino acid of the peptide. This coupling can be used to create an inactive proenzyme so that cleavage by FAP would cause a conformational change in the protein to activate it. For example, Pseudomonas toxin has a leader peptide sequence that is cleaved to activate the protein. Additionally the peptide could be incorporated into the Amino acid sequence of a protein toxin so that cleavage by FAP would liberate an inhibitory piece which would cause a conformational change in the protein to activate it. For example, proaerolysin, produced by Aeromonas hydrophila, is a protein toxin containing a binding domain, a toxin domain, an activation domain and an inhibitory domain. Incorporation of the FAP peptide sequence into the activation domain would generate a proaerolysin toxin that must be hydrolyzed by FAP to release the inhibitory domain to become activated. Additionally, the peptide sequence could be used to couple a drug to an antibody. The antibody could be coupled to the N-terminus of the peptide sequence, and the drug coupled to the carboxy terminus. The antibody would bind to a cell surface protein and tissue-specific protease present in the extracellular fluid could cleave the drug from the peptide linker.

The preferred amino acid sequence can be constructed to be highly specific for cleavage by FAP. In addition the peptide sequence can be constructed to be highly selective towards cleavage by FAP as compared to purified extracellular and intracellular proteases. Highly-specific FAP sequences can also be constructed that are also stable toward cleavage in human sera. Methods of selecting FAP substrates are disclosed infra.

In one embodiment, the present invention contemplates that the peptide sequences of the present invention (other than the cyclic peptides) are terminated with a CONH₂ group at the carboxy terminus. Although the present invention is not limited to any particular theory, it is believed that the CONH₂ group at the carboxy terminus aids in preventing the degradation of the peptide. In another embodiment, it is contemplated that the sequences of the present invention (other than the cyclic peptides) are terminated with a COOH group at the carboxy terminal. In yet another embodiment, it is contemplated that the sequences of the present invention (other than the cyclic peptides) are terminated with an NH₂ group at the N-terminus. In another embodiment, it is contemplated that the peptide sequences of the present invention may additionally comprise carbohydrate groups.

In one embodiment, the present invention contemplates that the peptides of the present invention are protease-resistant. In one embodiment, such protease-resistant peptides are peptides comprising protecting groups. In a preferred embodiment, endoprotease-resistance is achieved using peptides that comprise at least one D-amino acid.

As noted above, the present invention contemplates peptides that are protease-resistant. In one embodiment, such protease-resistant peptides are peptides comprising protecting groups. In a preferred embodiment, the present invention contemplates a peptide protected from exoproteinase degradation by N-terminal acetylation (“Ac”) and C-terminal amidation (—NH₂). The peptide is useful for in vivo administration because of its resistance to proteolysis.

In another embodiment, the present invention also contemplates peptides protected from endoprotease degradation by the substitution of L-amino acids in said peptides with their corresponding D-isomers. It is not intended that the present invention be limited to particular amino acids and particular D-isomers. In another embodiment, any of the amino acids may be substituted with the D form of the amino acid. In yet another embodiment of the present invention, more than one amino acid may be substituted with the D form of the amino acid. In still yet another embodiment, any of peptides contemplated by the present invention may have one or more amino acids substituted with the D form of the amino acid.

In one embodiment, the polypeptide is further modified to resist proteolytic degradation (e.g., upon in vivo delivery). For example, the polypeptide may be modified with protecting groups (e.g., wherein the amino acid sequence are N-terminally acetylated and C-terminally amidated).

### Labeling of Peptides and Substrates

Labeling of a peptide FAP substrate is typically conducted by mixing an appropriate reactive dye and the peptide to be conjugated in a suitable solvent in which both are soluble, using methods well-known in the art (Hermanson, Bioconjugate Techniques, (1996) Academic Press, San Diego, Calif.), followed by separation of the conjugate from any unconjugated starting materials or unwanted by-products. The dye conjugate can be stored dry or in solution for later use. The dyes may include a reactive linking group at one of the substituent positions for covalent attachment of the dye to another molecule. Reactive linking groups capable of forming a covalent bond are typically electrophilic functional groups capable of reacting with nucleophilic molecules, such as alcohols, alkoxides, amines, hydroxylamines, and thiols. Examples of reactive linking groups include succinimidyl ester, isothiocyanate, sulfonyl chloride, sulfonate ester, silyl halide, 2,6-dichlorotriazinyl, pentafluorophenyl ester, phosphoramidite, maleimide, haloacetyl, epoxide, alkylhalide, allyl halide, aldehyde, ketone, acylazide, anhydride, and iodoacetamide. An exemplary reactive linking group is N-hydroxysuccinimidyl ester (NHS) of a carboxyl group substituent of the dye. The NHS ester of the dye may be preformed, isolated, purified, and/or characterized, or it may be formed in situ and reacted with a nucleophilic group of a peptide, or the like.

Typically, the carboxyl form of the dye is activated by reacting with some combination of a carbodiimide reagent, e.g. dicyclohexylcarbodiimide, diisopropylcarbodiitnide, or a uronium reagent, e.g. EDC (N-ethyl-N′-(3-dimethylaminopropyl)carbodiimide), TSTU (O—(N-succinnimdyl)-N,N,N′,N′-tetramethyluronium tetrafluoroborate, HBTU (O-benzotriazol-1-yl)-N,N,N′,N′-tetramethyluronium hexafluorophosphate), or HATU (O-(7-azabenzotriazol-1-yl)-N,N,N′,N′-tetramethyluronium hexafluorophosphate), an activator, such as 1-hydroxybenzotriazole (HOBt), and N-hydroxysuccinimide to give the NHS ester of the dye. Other activating and coupling reagents include TBTU (2-(1H-benzotriazo-1-yl)-1-1,3,3-tetramethyluronium hexafluorophosphate), TFHH(N,N′,N″,N′″-tetramethyluronium 2-fluoro-hexafluorophosphate), PyBOP (benzotriazole-1-yl-oxy-tris-pyrrolidino-phosphonium hexafluorophosphate, EEDQ (2-ethoxy-1-ethoxycarbonyl-1,2-dihydro-quinoline), DCC (dicyclohexylcarbodiimide); DIPCDI (diisopropylcarbodiimide), MSNT (1-(mesitylene-2-sulfonyl)-3-nitro-1H-1,2,4-triazole, and aryl sulfonyl halides, e.g. triisopropylbenzenesulfonyl chloride.

Energy transfer dyes of a FRET pair include a donor dye which absorbs light at a first wavelength and emits excitation energy in response, an acceptor dye which is capable of absorbing the excitation energy emitted by the donor dye and fluorescing at a second wavelength in response. Dyes may be of any extended conjugation structure, such as a fluorescein, a rhodamine, a diazodiaryl-type, or a cyanine, many of which are commercially available (Molecular Probes Inc., Eugene Oreg.; Sigma Chemical Co., St. Louis, Mo.). A peptide may be labeled with a donor dye and an acceptor dye on opposite sides of the cleavage site of the peptide. Peptides can be labeled at the carboxyl terminus, the amino terminus, or an internal amino acid, e.g. cysteine or lysine side chain (U.S. Pat. No. 5,605,809).

The peptide may be substantially purified by preparative high performance liquid chromatography (Chiez, R. M. and F. Z. Regnier (1990) Methods Enzymol. 182:392-421). The composition of the synthetic peptides may be confirmed by amino acid analysis or by sequencing (Creighton, supra, pp. 28-53).

Methods which are well known to those skilled in the art may be used to construct expression vectors containing polynucleotides encoding the peptides of the invention and appropriate transcriptional and translational control elements. These methods include in vitro recombinant DNA techniques, synthetic techniques, and in vivo genetic recombination (Sambrook and Russell, supra, ch. 14, and 8; Ausubel et al., supra, ch. 1, 3, and 15).

### Substrate Specificity of FAP

Cleavage by FAP of peptide substrates can be detected and quantitated, for example, where the peptide is labeled with two moieties, a fluorescent reporter and quencher, which together undergo fluorescence resonance energy transfer (FRET). Cleavage of the FRET peptide releases fluorescence, e.g. ceases quenching which may be detected and quantitated. The fluorescence of the reporter may be partially or significantly quenched by the quencher moiety in an intact peptide. Upon cleavage of the peptide by a peptidase or protease, a detectable increase in fluorescence may be measured (Knight, C. (1995) “Fluorimetric Assays of Proteolytic Enzymes”, Methods in Enzymology, Academic Press, 248:18-34).

The substrate specificity of FAP may be measured, for example, with labeled peptide substrates (Edosada et al (2006) Jour. Biological Chem. 281(11):7437-7444). The degree of FAP enzymatic activity in tumors may be determined by an immunocapture assay with coumarin labelled substrates (Cheng et al (2005) Mol. Cancer Ther. 4(3):351-60; Cheng et al (2002) Cancer Res. 62:4767-4772).

Substrate specificity is demonstrated below in Tables 1, 2, and 3 and FIGS. 12 and 14.

### Prodrug Compositions

The invention also features prodrug compositions that consist of a therapeutic drug linked to a peptide containing a cleavage site that is specific for FAP or any enzyme that has the enzymatic activity of FAP. As noted above, the peptides of the invention can be used to target therapeutic drugs for activation within FAP producing tissue. The peptides that are useful in the prodrugs of the invention are those described above.

Peptidic prodrugs which are FAP cleavage substrates have been reported to be converted to cytotoxic or cytostatic metabolites by the sequence selective cleavage of FAP (U.S. Pat. No. 6,613,879; US 2003/021979; US 2003/0232742; US 2003/0055052; US 2002/0155565). Peptide proline-boronate protease inhibitors have been reported (Bachovchin et al (1990) Jour. Biol. Chem. 265(7):3738-3743; Flentke et al (1991) Proc. Natl. Acad. Sci. 88:1556-1559; Snow et al (1994) J. Amer. Chem. Soc. 116(24):10860-10869; Coutts et al (1996) J. Med. Chem. 39:2087-2094; U.S. Pat. No. 4,935,493; U.S. Pat. No. 5,288,707; U.S. Pat. No. 5,462,928; U.S. Pat. No. 6,825,169; WO 2003/092605; US 200410229820; WO 2005/047297). Cyclic boro-proline compounds are reported to be useful for oral administration (U.S. Pat. No. 6,355,614). An N-acetyl lysine proline boronate compound has been proposed as an antibacterial agent (U.S. Pat. No. 5,574,017).

The therapeutic drugs that may be used in the prodrugs of the invention include any drug that can be directly or indirectly linked to the FAP-specifically cleavable peptides of the invention. Preferred drugs are those containing a primary amine. The presence of the primary amine allows for formation of an amide bond between the drug and the peptide and this bond serves as the cleavage site for FAP. The primary amines may be found in the drugs as commonly provided, or they may be added to the drugs by chemical synthesis.

Certain therapeutic drugs contain primary amines and are among the preferred agents. These include the anthracycline family of drugs, vinca drugs (e.g., vinca alkloids such as vincristine, vinblastine, and etoposide), mitomycins, bleomycins, cytotoxic nucleoside analogs (e.g., 5-flurouracil, gemcitabine, and 5-azacytidine), the pteridine family of drugs, diynenes, podophyllotoxins, antiandrogens (e.g., biscalutamide, flutamide, nilutamide, and cyproterone acetate), antifolates (e.g., methotrexate), topoisomerase inhibitors (e.g., Topotecan and irinotecan), alkylating agents (e.g., cyclophosphamide, Cisplatinum, carboplatinum, and ifosfamide), taxanes (e.g., paclitaxel and docetaxel), and compounds which are useful as targeted radiation sensitizers (e.g., 5-flurouracil, gemcitabine, topoisomerase inhibitors, and cisplatinum). Additional particularly useful members of these classes include, for example, doxorubicin, daunorubicin, carminomycin, idarubicin, epirubicin, aminopterin, methopterin, mitomycin C, porfromycin, cytosine arabinoside, melphalan, vindesine, 6-mercaptopurine, and the like, including any therapeutic drug (e.g., any therapeutic drug used in the treatment of cancer, including prostate and/or breast cancer) known to those of skill in art.

Other therapeutic drugs are required to have primary amines introduced by chemical or biochemical synthesis, for example sesquiteipene-lactones such as thapsigargin, and thapsigargicin and many others know to those skilled in the art. The thapsigargins are a group of natural products isolated from species of the umbelliferous genus Thapsia. The term thapsigargins has been defined by Christensen, et al., Prog. Chem. Nat Prod., 71 (1997) 130-165. These derivatives contain a means of linking the therapeutic drug to carrier moieties, including peptides and antibodies. The peptides and antibodies can include those that specifically interact with antigens including FAP. The interactions can involve cleavage of the peptide to release the therapeutic analogs of sesquiterpene-γ-lactones. Particular therapeutic analogs of sesquitepene-γ-lactones, such as thapsigargins, are disclosed in U.S. Pat. Nos. 6,265,540 and 6,410,514, both of which are incorporated herein in their entireties.

Thapsigargin is a sesquiterpene-γ-lactone having the structure disclosed in International Publication No. WO 98/52966. Primary amines can be placed in substituent groups pendant from either C-2 or C-8 carbon (carbons are numbered as described in International Publication No. WO 98/52966). Preferred primary amine containing thapsigargin analogs that can be coupled to the peptides described above include those described previously by the inventors (“Tissue Specific Prodrug” International Patent Application PCT/US98/10285, published as International Publication No. WO 98/52966, corresponding to U.S. Ser. Nos. 60/047,070 and 60/080,046, filed May 19, 1997 and Mar. 30, 1998). These primary amine-containing analogs have non-specific toxicity toward cells. This toxicity is measured as the toxicity needed to kill 50% of clonogenic cells (LC₅₀). The LC50 of the analogs of this invention is desirably at most 10 μM, preferably at most 2 μM and more preferably at most 200 nM of analog.

For example, thapsigargins with alkanoyl, alkenoyl, and arenoyl groups at carbon 8 or carbon 2, can be employed in the practice of the invention disclosed herein. Groups such as CO—(CH═CH)ₙ₁—(CH2)ₙ₂-Ar—NH₂, CO—(CH₂)ₙ₂—(CH═CH)ₙ₁—Ar—NH₂, CO—(CH2)ₙ₂-(CH═CH)ₙ₁—CO—NH—Ar—NH₂ and CO—(CH═CH)ₙ₁—(CH2)ₙ₂-CO—NH—Ar—NH₂ and substituted variations thereof can be used as carbon 8 substituents, where n1 and n2 are from 0 to 5, and Ar is any substituted or unsubstituted aryl group. Substituents which may be present on Ar include short and medium chain alkyl, alkanoxy, aryl, aryloxy, and alkenoxy groups, nitro, halo, and primary secondary or tertiary amino groups, as well as such groups connected to Ar by ester or amide linkages.

In other embodiments of thapsigargin analogs, these substituent groups are represented by unsubstituted, or alkyl-, aryl-, halo-, alkoxy-, alkenyl-, amino-, or amino-substituted CO—CH2)n3-NH2, where n3 is from 0 to 15, preferably 3-15, and also preferably 6-12. Particularly preferred substituent groups within this class are 6-aminohexanoyl, 7-aminoheptanoyl, 8-aminooctanoyl, 9-aminononanoyl, 10-aminodecanoyl, 11-aminoundecanoyl, and 12-aminododecanoyl. These substituents are generally synthesized from the corresponding amino acids, 6-aminohexanoic acid, and so forth. The amino acids are N-terminal protected by standard methods, for example Boc protection. Dicyclohexylcarbodiimide (DCCI)-promoted coupling of the N-terminal protected substituent to thapsigargin, followed by standard deprotection reactions produces primary amine-containing thapsigargin analogs.

The substituents can also carry primary amines in the form of an amino amide group attached to the alkanoyl-, alkenoyl-, or arenoyl substituents. For example, C-terminal protection of a first amino acid such as 6-aminohexanoic acid and the like, by standard C-terminal protection techniques such as methyl ester formation by treatment with methanol and thionyl chloride, can be followed by coupling the N-terminal of the first amino acid with an N-protected second amino acid of any type.

In a preferred embodiment, the thapsigargin analog or derivative is 8-O-(12-[LP-leucinoylamino]dodecanoyl)8-O-debutanoylthapsigargin, also referred to herein as “L12ADT”.

The peptide and therapeutic drug are linked directly or indirectly (by a linker) through the carboxy terminus of the peptide. The site of attachment on the therapeutic drug must be such that, when coupled to the peptide, the non-specific toxicity of the drug is substantially inhibited. Thus the prodrugs should not be significantly toxic.

The peptides and prodrugs of the invention may also comprise groups which provide solubility to the peptide or prodrug as a whole in the solvent in which the peptide or prodrug is to be used. Most often the solvent is water. This feature of the invention is important in the event that neither the peptide nor the therapeutic drug is soluble enough to provide overall solubility to the peptide or prodrug. These groups include polysaccharides or other polyhydroxylated moieties. For example, dextan, cyclodextrin, starch and derivatives of such groups may be included in the peptide or prodrug of the invention. In a preferred embodiment, the group which provides solubility to the peptide or prodrug is a polymer, e.g., polylysine or polyethylene glycol (PEG).

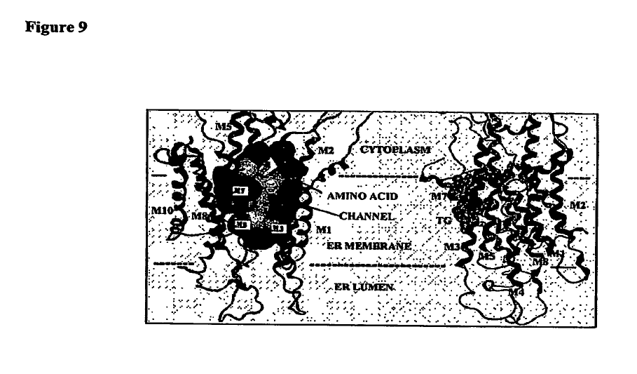

FIG. 9 shows a model of TG analog containing long hydrophobic side chain coupled to amino acid showing hydrophobic side chain in channel and amino acid interacting with the cytoplasm outside of the channel.

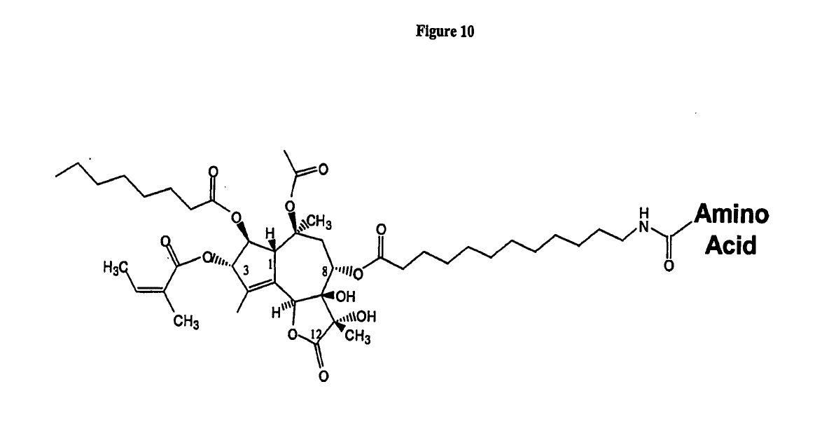

FIG. 10 shows a chemical structure of thapsigargin analog modified in O-8 position with 12-aminododecanoyl side chain coupled to carboxyl-group of an amino acid.

Advantages of these agents for targeting cells disclosed herein within the stroma because they are able to kill cells via a proliferation independent mechanism.

An example of a compound portion of the prodrug is thapsigargin, a non-specific highly potent cytotoxin with an average IC₅₀ of 10⁻¹⁰ M. In comparison, the commonly used antiproliferative chemotherapeutic agent paclitaxel had an average IC₅₀ of 10⁻⁸ M in this same assay. Thapsigargin is highly potent killer of all cell lines tested whether they were malignant or normal (e.g., fibroblasts, osteoblasts, endothelial cells, etc.).

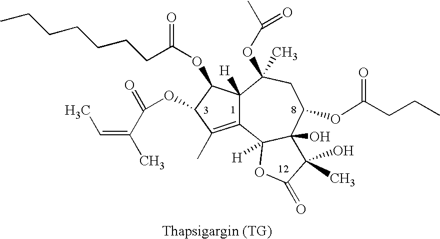

Thapsigargin, however, has a unique mechanism of cytotoxicity. Without wishing to be bound by an particular scientific theory, it is a potent inhibitor of the Sarcoplasmic/Endoplasrnic Reticulum Calcium ATPase pump which is a critical intracellular protein required by all cells to maintain metabolic viability (33). Inhibition of the SERCA pump by thapsigargin leads to sustained elevation of intracellular calcium which activates both ER-stress and mitochondrial apoptotic pathways (33)

### Pharmaceutical Formulations

Compounds of the present invention are useful for treating diseases, conditions and/or disorders modulated/influenced/exacerbated by FAP. Therefore, an embodiment of the present invention is a pharmaceutical composition, e.g. formulation, comprising a therapeutically effective amount of a compound of the present invention and a pharmaceutically acceptable excipient, diluent or carrier.

A typical formulation is prepared by mixing a compound of the present invention and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (e.g., a compound of the present invention or pharmaceutical composition thereof) or aid in the manufacturing of the pharmaceutical product (e.g., medicament).

The formulations may be prepared using conventional dissolution and mixing procedures. For example, the bulk drug substance (e.g., compound of the present invention or stabilized form of the compound (e.g., complex with a cyclodextrin derivative or other known complexation agent)) is dissolved in a suitable solvent in the presence of one or more of the excipients described above. The compound of the present invention is typically formulated into pharmaceutical dosage forms to provide an easily controllable dosage of the drug and to enable patient compliance with the prescribed regimen.

The pharmaceutical composition (or formulation) for application may be packaged in a variety of ways depending upon the method used for administering the drug. Generally, a kit or article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Pharmaceutical, formulations of therapeutic compounds of the invention may be prepared for various routes and types of administration. A compound having the desired degree of purity is optionally mixed with pharmaceutically acceptable diluents, carriers, excipients or stabilizers (Remington's Pharmaceutical Sciences (1980) 16th edition, Osol, A. Ed.), in the form of a lyophilized formulation, milled powder, or an aqueous solution. Formulation may be conducted by mixing at ambient temperature at the appropriate pH, and at the desired degree of purity, with physiologically acceptable carriers, e.g., carriers that are non-toxic to recipients at the dosages and concentrations employed. The pH of the formulation depends mainly on the particular use and the concentration of compound, but may range from about 3 to about 8. Formulation in an acetate buffer at pH 5 is a suitable embodiment.

The compound for use herein is preferably sterile. The compound ordinarily will be stored as a solid composition, although lyophilized formulations or aqueous solutions are acceptable.

The pharmaceutical compositions of the invention will be formulated, dosed, and administered in a fashion, e.g. amounts, concentrations, schedules, course, vehicles, and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the compound to be administered will be governed by such considerations, and is the minimum amount necessary to prevent, ameliorate, or treat the coagulation factor mediated disorder. Such amount is preferably below the amount that is toxic to the host or renders the host significantly more susceptible to bleeding.

Acceptable diluents, carriers, excipients, and stabilizers are nontoxic to recipients at the dosages and concentrations employed, and include buffers such as phosphate, citrate, an other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-resol); low molecular weight (less than about 10 residues) polypeptides; proteins, such as serum album gelatin, or immunoglobulins; hydrophilic polymers such as polyvinylpyrrolidone; amino acids such as glycine, glutamine, asparagine, histidine, arginine, or lysine; monosaccharides disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). The active pharmaceutical ingredients may also be entrapped in microcapsules prepared, for example, coacervation techniques or by interfacial polymerization, for example, hydroxymethylcellulose or gelatin-microcapsules and poly-(methylmethacylate)microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the compound of the invention, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gama-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations include those suitable for the administration routes detailed herein. The formulations may be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier that constitutes one or more accessory ingredients. In general the formulations are prepared by uniformly and intimately bringing into association the active ingredient with liquid carriers or finely divided solid carriers or both, and then, if necessary, shaping the product.

Compressed tablets may be prepared by compressing in a suitable machine the active ingredient in a fee-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Tablets, troches, lozenges, aqueous or oil suspensions, dispersible powders or granules, emulsions, hard or soft capsules, e.g. gelatin capsules, syrups or elixirs may be prepared for oral use. Formulations of a compounds intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipients suitable for manufacture of tablets are acceptable. Excipients may include, for example, calcium carbonate, sodium carbonate, lactose, calcium phosphate, sodium phosphate, mannitol, crospovidone, polysorbate 80, hydroxypropyl methylcellulose, colloidal silicon dioxide, microcrystalline cellulose, sodium starch glycolate, simethicone, polyethylene glycol 6000, sucrose, magnesium carbonate, titanium dioxide, methylparaben, and polyvinyl alcohol. Excipients may also include granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

For use in the eye or other external tissues e.g. mouth and skin, the formulations are preferably applied as a topical ointment or cream containing the active ingredient(s) in an amount of, for example, 0.075 to 20% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include a polyhydric alcohol, e.g. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound that enhances absorption or penetration of the active ingredient through the skin or other affected areas. Examples of such dermal penetration enhancers include dimethyl sulfoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier that acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base that forms the oily dispersed phase of the cream formulations. Emulgents and emulsion stabilizers suitable for use in the formulation of the invention include Tween™ 60, Span™ 80, cetostearyl alcohol, benzyl alcohol, myristyl alcohol, glyceryl mono-stearate and sodium lauryl sulfate.

Aqueous suspensions of the invention contain the active materials in admixture with excipients suitable for the manufacture of aqueous suspensions. Such excipients include a suspending agent, such as sodium carboxymethylcellulose, croscaemellose, povidone, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

The pharmaceutical composition of the compounds may be in the form of a sterile injectable preparation, such as a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10% particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient Formulations suitable for aerosol or dry powder administration may be prepared according to conventional methods and may be delivered with other therapeutic agents such as compounds heretofore used in the treatment or prophylaxis of HIV infections as described below.

Formulations suitable for vaginal administration may be presented as pessaries, tampons, creams, gels, pastes, foams or spray formulations containing in addition to the active ingredient such carriers as are known in the art to be appropriate.

The formulations may be packaged in unit-dose or multi-dose containers, for example pills, sealed ampoules, vials, and blister packs. Formulations may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The invention further provides veterinary compositions comprising at least one active ingredient as above defined together with a veterinary carrier therefore. Veterinary carriers are materials useful for the purpose of administering the composition and may be solid, liquid or gaseous materials that are otherwise inert or acceptable in the veterinary art and are compatible with the active ingredient. These veterinary compositions may be administered parenterally, orally or by any other desired route.

Other delivery systems can include timed release, delayed release or sustained release delivery systems. Such systems can avoid repeated administrations of the agent of the invention, increasing convenience to the subject and the physician. Many types of release delivery systems are available and known to those of ordinary skill in the art. They include the above-described polymeric systems, as well as polymer base systems such as poly(lactide-glycolide), copolyoxalates, polycaprolactones, polyestermides, polyorhoesters, polybydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, for example: (a) erosional systems in which the agent is contained in a form within a matrix such as those described in U.S. Pat. Nos. 4,452,775, 4,675,189 and 5,736,152 and (b) diffusional systems in which an active component permeates at a controlled rate from a polymer such as described in U.S. Pat Nos. 3,854,480, 5,133,974 and 5,407,686. In addition, pump-based hardware delivery systems can be used, some of which are adapted for implantation.

In still other embodiments, the agent is targeted to a site of abnormal cell proliferation, such as, a tumor, through the use of a targeting compound specific for a particular tissue or tumor type. The agents of the invention may be targeted to primary or in some instances, secondary (e.g., metastatic) lesions through the use of targeting compounds that preferentially recognize a cell surface marker. The targeting compound may be directly conjugated to the agents of the invention via a covalent linkage. The agent may be indirectly conjugated to a targeting compound via a linker. Alternatively, the targeting compound may be conjugated or associated with an intermediary compound such as, for example, a liposome within which the agent is encapsulated. Liposomes are artificial membrane vessels that are useful as a delivery vector in vivo or in vitro. It has been shown that large unilamellar vessels (LUV), which range in size from 0.2-4.0 μm can encapsulate large macromolecules. Liposomes may be targeted to a particular tissue, such as the vascular cell wall, by coupling the liposome to a specific ligand such as a monoclonal antibody, sugar, glycolipid, or protein. Liposomes are commercially available from Gibco BRL, for example, as LIPOFECIIN.™. and LlPOFECTACE™, which are formed of cationic lipids such as N-[1,2,3 dioleyloxy)-propyl]-N,N,N-trimethylammonium chloride (DOTMA) and dimethyl dioctadecylanmonium bromide (DDAB). Methods for making liposomes are well known in the art and have been described in many publications. Liposomes also have been reviewed by Gregoriadis, G. in Trends in Biotechnology, V. 3, p. 235-241 (1985). In still other embodiments, the targeting compound may be loosely associated with the agents of the invention, such as within a microparticle comprising a polymer, the agent of the invention and the targeting compound.

### Metabolites

Also falling within the scope of this invention are the in vivo metabolic products of the compounds described herein, to the extent such products are novel and unobvious over the prior art. Such products may result for example from the oxidation, reduction, hydrolysis, amidation, deamidation, esterification, deesterification, enzymatic cleavage, and the like, of the administered compound. Accordingly, the invention includes novel and unobvious compounds produced by a process comprising contacting a compound of this invention with a mammal for a period of time sufficient to yield a metabolic product thereof.

Metabolite products typically are identified by preparing a radiolabelled (e.g. ¹⁴C or ³H) isotope of a compound of the invention, administering it parenterally in a detectable dose (e.g. greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g. by MS, LC/MS or NMR analysis. In general, analysis of metabolites is done in the same way as conventional drug metabolism studies well known to those skilled in the art. The conversion products, so long as they are not otherwise found in vivo, are useful in diagnostic assays for therapeutic dosing of compounds of the invention.

### Dosages

The prodrugs of the invention, or compositions thereof, will generally be used in an amount effective to achieve the intended purpose. Of course, it is to be understood that the amount used will depend on the particular application.

For use to treat or prevent tumor or target cell growth or diseases related thereto, the prodrugs of the invention, or compositions thereof, are administered or applied in a therapeutically effective amount By therapeutically effective amount is meant an amount effective to ameliorate the symptoms of, or ameliorate, treat or prevent tumor or target cell growth or diseases related thereto. Determination of a therapeutically effective amount is well within the capabilities of those skilled in the art, especially in light of the detailed disclosure provided herein.

For systemic administration, a therapeutically effective dose can be estimated initially from in vitro assays. For example, a dose can be formulated in animal models to achieve a circulating prodrug concentration range that includes the 150 as determined in cell culture (e.g., the concentration of test compound that is lethal to 50% of a cell culture), the MIC, as determined in cell culture (e.g., the minimal innaubitory concentration for growth) or the I.sub.100 as determined in cell culture (e.g., the concentration of peptide that is lethal to 100% of a cell culture). Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

The amount of prodrug administered will, of course, be dependent on the subject being treated, on the subject's weight, the severity of the affliction, the manner of administration and the judgment of the prescribing physician.

The antitumoral therapy may be repeated intermittently. The therapy may be provided alone or in combination with other drugs, such as for example other antineoplastic entities or other pharmaceutically effective entities.

### Toxicity

Preferably, a therapeutically effective dose of the prodrugs described herein will provide therapeutic benefit without causing substantial toxicity.

Toxicity of the prodrugs described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. Compounds which exhibit high therapeutic indices are preferred. The data obtained from these cell culture assays and animal studies can be used in formulating a dosage range that is not toxic for use in human. The dosage of the prodrugs described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition. (See, e.g., Fingi et al., 1975, In: The Pharmacological Basis of Therapeutics, Ch.1, p.1).

A variety of expression vector/host systems may be utilized to contain and express polynucleotides encoding the polypeptides and prodrugs of the invention. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems (Sambrook and Russell, supra; Ausubel et al., supra; Van Heeke, G. and S. M. Schuster (1989) J. Biol. Chem. 264:5503-5509; Engelhard, E. K et al. (1994) Proc. Natl. Acad Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology 1 (1992) McGraw Hill, New York N.Y., pp. 191-196; Logan, J. and T. Shenk (1984) Proc. Natl. Acad. Sci. USA 81:3655-3659; Harrington, J. J. et al. (1997) Nat. Genet. 15:345-355). Expression vectors derived from retroviruses, adenoviruses, or herpes or vaccinia viruses, or from various bacterial plasmids, may be used for delivery of polynucleotides to the targeted organ, tissue, or cell population (Di Nicola, M. et al. (1998) Cancer Gen. Ther. 5:350-356; Yu, M. et al. (1993) Proc. Natl. Acad. Sci. USA 90:6340-6344; Buller, R. M. et al. (1985) Nature 317:813-815; McGregor, D. P. et al. (1994) Mol. Immunol. 31:219-226; Verma, I. M. and N. Somia (1997) Nature 389:239-242). The invention is not limited by the host cell employed. Expression systems include, for example, bacterial systems (Van Heeke, G. and S. M. Schuster (1989) J. Biol. Chem. 264:5503-5509); yeast expression systems (Ausubel et al., supra; Bitter, G. A. et al. (1987) Methods Enzymol. 153:516-544; Scorer, C. A. et al. (1994) Bio/Technology 12:181-184); plant systems (Takamatsu, N. (1987) EMBO J. 6:307-311; Coruzzi, G. et al. (1984) EMBO J. 3:1671-1680; Broglie, R. et al. (1984) Science 224:838-843; Winter, J. et al. (1991) Results Probl. Cell Differ. 17:85-105); mammalian cells (Logan, J. and T. Shenk (1984) Proc. Natl. Acad. Sci. USA 81:3655-3659); and human artificial chromosomes (HACs) (Harrington, J. J. et al. (1997) Nat Genet. 15:345-355).

### Articles of Manufacture

In another embodiment of the invention, an article of manufacture, or “kit”, containing materials useful for the treatment of the disorders described above is provided. The article of manufacture comprises a container and a label or package insert on or associated with the container. Suitable containers include, for example, bottles, vials, syringes, blister pack, etc. The containers may be formed from a variety of materials such as glass or plastic. The container holds a compound or formulation thereof effective for treating the condition and may have a sterile access port (for example the container may be an intravenous solution bag or a vial having a stopper pierceable by a hypodermic injection needle). At least one active agent in the composition is a compound of the invention. The label or package insert indicates that the composition is used for treating the condition of choice, such as cancer. In one embodiment, the label or package insert includes instructions for use and indicates that the composition comprising a compound of the invention and can be used to treat a hyperproliferative disorder.

The article of manufacture may comprise (a) a first container with a compound of the invention contained therein; and (b) a second container with a second pharmaceutical formulation contained therein, wherein the second pharmaceutical formulation comprises a second compound with anti-hyperproliferative activity. The article of manufacture in this embodiment of the invention may further comprise a package insert indicating that the first and second compounds can be used to treat patients a hyperproliferative disorder, such as cancer. Alternatively, or additionally, the article of manufacture may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

According to another aspect of the invention, a kit is provided. The kit is a package which houses a container which contains an agent of the invention and also houses instructions for administering the agent of the invention to a subject having a condition characterized by an abnormal mammalian cell proliferation. The kit may optionally also contain one or more other anti-proliferative compounds or one or more anti-angiogenic compounds for use in combination therapies as describes herein.

In still another aspect of the invention, kits for administration of an agent of the invention to a subject is provided. The kits include a container containing a composition which includes at least one agent of the invention, and instructions for administering the at least one agent to a subject having a condition characterized by an abnormal mammalian cell proliferation in an amount effective to inhibit proliferation. In certain embodiments, the container is a container for intravenous administration. In other embodiments the agent is provided in an inhaler. In still other embodiments, the agent is provided in a polymeric matrix or in the form of a liposome. In yet other embodiments, kits are provided for the administration of an agent of the invention to a subject having an abnormal mammalian cell mass for the purpose of inhibiting angiogenesis in the cell mass. In these latter kits, the agent is provided in an amount effective to inhibit angiogenesis along with instructions for use in subjects in need of such treatment.

### Methods of Treating

The invention also provides methods of treatment of treating FAP-producing cell proliferative disorders of the invention with the prodrugs of the invention. The prodrugs of the invention and/or analogs or derivatives thereof can be administered to any host, including a human or non-human animal, in an amount effective to treat a disorder.

The prodrugs of the invention can be administered parenterally by injection or by gradual infusion over time. The prodrugs can be administered intravenously, intraperitoneally, intramuscularly, subcutaneously, intracavity, or transdermally. Preferred methods for delivery of the prodrug include intravenous or subcutaneous administration. Other methods of administration, as well as dosing regimens, will be known to those skilled in the art.

According to one aspect of the invention, a method for treating a subject having a condition characterized by an abnormal mammalian cell proliferation is provided. As used herein, subject means a mammal including humans, nonhuman primates, dogs, cats, sheep, goats, horses, cows, pigs and rodents. An abnormal mammalian cell proliferation disorder or condition, as used herein, refers to a localized region of cells (e.g., a tumor) that exhibit an abnormal (e.g., increased) rate of division as compared to their normal tissue counterparts.

Conditions characterized by an abnormal mammalian cell proliferation, as used herein, include, for example, to conditions involving solid tumor masses of benign, pre-malignant or malignant character. Although not wishing to be bound by a particular theory or mechanism, some of these solid tumor masses arise from at least one genetic mutation, some may display an increased rate of cellular proliferation as compared to the normal tissue counterpart, and still others may display factor independent cellular proliferation. Factor independent cellular proliferation is an example of a manifestation of loss of growth control signals that some, if not all, tumors or cancers undergo.

In one aspect, the invention provides a method for treating subjects having a condition characterized by an abnormal epithelial cell proliferation. Epithelial cells are cells occurring in one or more layers which cover the entire surface of the body and which line most of the hollow structures of the body, excluding the blood vessels, lymph vessels, and the heart interior which are lined with endothelium, and the chest and abdominal cavities which are lined with mesothelium.

Another category of conditions characterized by abnormal epithelial cell proliferation is tumors of epithelial origin. FAP-α has been observed in tumors of epithelial origin. Thus, in one aspect, the invention provides a method for treating subjects having epithelial tumors. Epithelial tumors are known to those of ordinary skill in the art and include, for example, benign and premalignant epithelial tumors, such as breast fibroadenoma and colon adenoma, and malignant epithelial tumors. Malignant epithelial tumors include primary tumors, also referred to as carcinomas, and secondary tumors, also referred to as metastases of epithelial origin. Carcinomas intended for treatment with the methods of the invention include, for example, acinar carcinoma, acinous carcinoma, alveolar adenocarcinoma (also called adenocystic carcinoma, adenomyoepithelioma, cribriform carcinoma and cylindroma), carcinoma adenomatosum, adenocarcinoma, carcinoma of adrenal cortex, alveolar carcinoma, alveolar cell carcinoma (also called bronchiolar carcinoma, alveolar cell tumor and pulmonary adenomatosis), basal cell carcinoma, carcinoma basocellulare (also called basaloma, or basiloma, and hair matrix carcinoma), basaloid carcinoma, basosquamous cell carcinoma, breast carcinoma, bronchioalveolar carcinoma, bronchiolar carcinoma, bronchogenic carcinoma, cerebriform carcinoma, cholangiocellular carcinoma (also called cholangioma and cholangiocarcinoma), chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliale adenoides, carcinoma exulcere, carcinoma fibrosum, gelatiniform carcinoma, gelatinous carcinoma, giant cell carcinoma, gigantocellulare, glandular carcinoma, granulosa cell carcinoma, hair-matix carcinoma, hematoid carcinoma, hepatocellular carcinoma (also called hepatoma, malignant hepatoma and hepatocarcinoma), Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, lenticular carcinoma, carcinoma lenticulare, lipomatous carcinoma, lymphoepithelial carcinoma, carcinoma mastitoides, carcinoma medullare, medullary carcinoma, carcinoma melanodes, melanotic carcinoma, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, carcinoma nigrum, oat cell carcinoma, carcinoma ossificans, osteoid carcinoma, ovarian carcinoma, papillary carcinoma, periportal carcinoma, preinvasive carcinoma, prostate carcinoma, renal cell carcinoma of kidney (also called adenocarcinoma of kidney and hypernephoroid carcinoma), reserve cell carcinoma, carcinoma sarcomatodes, scheinderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squanous cell carcinoma, string carcinoma, carcinoma telangiectaticum, carcinoma telangiectodes, transitional cell carcinoma, carcinoma tuberosum, tuberous carcinoma, verrucous carcinoma, carcinoma vilosum. In preferred embodiments, the methods of the invention are used to treat subjects having cancer of the breast, cervix, ovary, prostate, lung, colon and rectum, pancreas, stomach or kidney.

Other conditions characterized by an abnormal mammalian cell proliferation to be treated by the methods of the invention include sarcomas. Sarcomas are rare mesenchymal neoplasms that arise in bone and soft tissues. Different types of sarcomas are recognized and these include: liposarcomas (including myxoid liposarcomas and pleiomorphic liposarcomas), leiomyosarcomas, rhabdomyosarcomas, malignant peripheral nerve sheath tumors (also called malignant schwannomas, neurofibrosarcomas, or neurogenic sarcomas), Ewing's tumors (including Ewing's sarcoma of bone, extraskeletal [not bone] Ewing's sarcoma, and primitive neuroectodermal tumor [PNET]), synovial sarcoma, angiosarcomas, hemangiosarcomas, lymphangiosarcomas, Kaposi's sarcoma, hemangioendothelioma, fibrosarcoma, desmoid tumor (also called aggressive fibromatosis), dermatofibrosarcoma protuberans (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) (also known as GI stromal sarcoma), osteosarcoma (also known as osteogenic sarcoma)-skeletal and extraskeletal, and chondrosarcoma.

The methods of the invention are also directed towards the treatment of subjects with melanoma. Melanomas are tumors arising from the melanocytic system of the skin and other organs. Examples of melanoma include lentigo maligna melanoma, superficial spreading melanoma, nodular melanoma, and acral lentiginous melanoma.

Other conditions characterized by an abnormal mammalian cell proliferation are cancers including, for example, biliary tract cancer, endometrial cancer, esophageal cancer, gastric cancer, intraepithelial neoplasms, including Bowen's disease and Paget's disease, liver cancer, oral cancer, including squamous cell carcinoma, sarcomas, including fibrosarcoma and osteosarcoma, skin cancer, including melanoma, Kaposi's sarcoma, testicular cancer, including germinal tumors (seminoma, non-seminoma (teratomas, choriocarcinomas)), stromal tumors and germ cell tumors, thyroid cancer, including thyroid adenocarcinoma and medullar carcinoma, and renal cancer including adenocarcinoma and Wilms tumor.

According to other aspects of the invention, a method is provided for treating a subject having an abnormal proliferation originating in bone, muscle or connective tissue. Exemplary conditions intended for treatment by the method of the invention include primary tumors (e.g., sarcomas) of bone and connective tissue.

The methods of the invention are also directed towards the treatment of subjects with metastatic tumors. In some embodiments, the metastatic tumors are of epithelial origin. Carcinomas may metastasize to bone, as has been observed with breast cancer, and liver, as is sometimes the case with colon cancer. The methods of the invention are intended to treat metastatic tumors regardless of the site of the metastasis and/or the site of the primary tumor. In preferred embodiments, the metastases are of epithelial origin.

### Combination Therapy Methods

Compounds of the invention may be combined in a pharmaceutical combination formulation, or dosing regimen as combination therapy, with a second compound that has anti-hyperproliferative properties or that is useful for treating a hyperproliferative disorder (e.g. cancer). The second compound of the pharmaceutical combination formulation or dosing regimen preferably has complementary activities to the compounds of the invention such that they do not adversely affect the other(s). Such molecules are suitably present in combination in amounts that are effective for the purpose intended.

The combination therapy may be administered as a simultaneous or sequential regimen. When administered sequentially, the combination may be administered in two or more administrations. The combined administration includes coadministration, using separate formulations or a single pharmaceutical formulation, and consecutive administration in either order, wherein preferably there is a time period while both (or all) active agents simultaneously exert their biological activities. Suitable dosages for any of the above coadministered agents are those presently used and may be lowered due to the combined action (synergy) of the newly identified agent and other chemotherapeutic agents or treatments.

The combination therapy may provide “synergy” and prove “synergistic”, e.g. the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g. by different injections in separate syringes. In general, during alternation therapy, an effective dosage of each active ingredient is administered sequentially, e.g. serially, whereas in combination therapy, effective dosages of two or more active ingredients are administered together.

As an example, the agent may be administered in combination with surgery to remove an abnormal proliferative cell mass. As used herein, “in combination with surgery” means that the agent may be administered prior to, during or after the surgical procedure. Surgical methods for treating epithelial tumor conditions include intra-abdominal surgeries such as right or left hemicolectomy, sigmoid, subtotal or total colectomy and gastrectomy, radical or partial mastectomy, prostatectomy and hysterectomy. In these embodiments, the agent may be administered either by continuous infusion or in a single bolus. Administration during or immediately after surgery may include a lavage, soak or perfusion of the tumor excision site with a pharmaceutical preparation of the agent in a pharmaceutically acceptable carrier. In some embodiments, the agent is administered at the time of surgery as well as following surgery in order to inhibit the formation and development of metastatic lesions. The administration of the agent may continue for several hours, several days, several weeks, or in some instances, several months following a surgical procedure to remove a tumor mass.

The subjects can also be administered the agent in combination with non-surgical anti-proliferative (e.g., anti-cancer) drug therapy. In one embodiment, the agent may be administered in combination with an anti-cancer compound such as a cytostatic compound. A cytostatic compound is a compound (e.g., a nucleic acid, a protein) that suppresses cell growth and/or proliferation. In some embodiments, the cytostatic compound is directed towards the malignant cells of a tumor. In yet other embodiments, the cytostatic compound is one that inhibits the growth and/or proliferation of vascular smooth muscle cells or fibroblasts.

Suitable anti-proliferative drugs or cytostatic compounds to be used in combination with the agents of the invention include anti-cancer drugs. Anti-cancer drugs are well known and include: Acivicin; Aclarubicin; Acodazole Hydrochloride; Acronine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfin; Cactinomycin; Calusterone; Caracemide; Carbetimer; Carboplatin; Canmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatreate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-Ia; Interferon Gamma-Ib; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicainycin; Plomestane; Porfimer Sodium; Porfiromycin; Prednimustine; Procarbazine Hydrochloride; Puromycin; Puromycin Hydrochloride; Pyrzofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Taxotere; Tecogalan Sodium; Tegafur; Teloxantrne Hydrochloride; Temoporfin; Teniposide; Teroxirone; Testolactone; Thiamiprine; Thioguanine; Thiotepa; Tiazofuirin; Tirapazamine; Topotecan Hydrochloride; Torermifene Citrate; Trestolone Acetate; Tricintbine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

According to the methods of the invention, the agents of the invention may be administered prior to, concurrent with, or following the other anti-cancer compounds. The administration schedule may involve administering the different agents in an alternating fashion. In other embodiments, the agent may be delivered before and during, or during and after, or before and after treatment with other therapies. In some cases, the agent is administered more than 24 hours before the administration of the other anti-proliferative treatment. In other embodiments, more than one anti-proliferative therapy may be administered to a subject. For example, the subject may receive the agents of the invention, in combination with both surgery and at least one other anti-proliferative compound. Alternatively, the agent may be administered in combination with more than one anti-cancer drug.

### Method of Screening Tissue and Determining FAP Activity

In another aspect the invention provides a method of detecting FAP-producing tissue using peptides of the invention, as described above. The method is carried out by contacting a detectably labeled peptide of the invention with target tissue for a period of time sufficient to allow FAP to cleave the peptide and release the detectable label. The detectable label is then detected. The level of detection is compared to that of a control sample not contacted with the target tissue. Many varieties of detectable labels are available, including optically based labels such as chromophoric, chemiluminescent, fluoresecent or phosphorescent labels and radioactive labels, such as alpha, beta, or gamma emitting labels. In addition a peptide label consisting of an amino acid sequence can be utilized for detection such that release of the peptide label by FAP proteolysis can be detected by high pressure liquid chromatography. The peptide sequences of the invention can also be incorporated into the protein sequence of a fluorescent protein such that cleavage of the incorporated FAP specific sequence by FAP results in either an increased or decreased fluorescent signal that can be measured using the appropriate fluorometric measuring instrument. In a preferred embodiment, the peptide comprises a fluorescent label at its carboxy terminus (e.g., L(ABZ)), and a quencher at its amino terminus (e.g., a nitrotyrosine residue), such that the label is quenched when the peptide is intact, and fluorescent when the peptide is cleaved.

The invention provides a method for detecting a cell proliferative disorder that comprises contacting a FAP-specific peptide with a cell suspected of producing FAP. The FAP reactive peptide is labeled by a compound so that cleavage by FAP can be detected. For purposes of the invention, a peptide specific for FAP may be used to detect the level of enzymatically active FAP in biological tissues such as saliva, blood, urine, and tissue culture media. In an embodiment of the method a specific FAP inhibitor is used to confirm that the activity being measured is solely due to peptide cleavage by FAP and not secondary to non-specific cleavage by other proteases present in the biological tissue being assayed. Examples of FAP inhibitors that can be employed in the method include the addition of zinc ions, or the addition of FAP specific antibodies that bind to the catalytic site of FAP thereby inhibiting enzymatic activity of FAP.

Methods also include the use of synthetic or recombinant produces collagen I and gelatins. The advantage of using synthetic or recombinant proteins is discussed infra.

### Method of Screening Prodrugs

The invention also provides a method of selecting potential prodrugs for use in the invention. The method generally comprises contacting prodrugs of the invention with FAP-producing tissue and non-FAP producing tissue in a parallel experiment. The prodrugs which exert toxic effects in the presence of FAP-producing tissue, but not in the presence of non-FAP producing tissue are suitable for the uses of the invention.

### Method of Identifying Substrates for FAP

The invention also provides a method for identifying peptide sequences which are substrates for FAP. The method generally comprises generating a library of random peptides, incubating the peptides with FAP, detecting the peptides that are cleaved by FAP, and determining the sequence of the cleaved peptides. In a preferred embodiment, the peptides comprise a label that is undetectable when the peptides are intact, but detectable when they are cleaved. In a further preferred embodiment, the peptides are attached to a mechanical support (e.g., a bead), and the cleaved peptides can be separated manually from the intact peptides. More specific details for performing the method may be found in the Examples below.

### Methods of Making Prodrug Compounds

The invention provides a method of producing the prodrugs of the invention. This method involves linking a therapeutically active drug to a peptide of the invention described above. In certain embodiments the peptide is linked directly to the drug; in other embodiments the peptide is indirectly linked to the drug via a linker. In certain embodiments, the carboxy terminus of the peptide is used for linking. The therapeutic drug contains a primary amine to facilitate the formation of an amide bond with the peptide. Many acceptable methods for coupling carboxyl and amino groups to form amide bonds are know to those skilled in the art.

The peptide may be coupled to the therapeutic drug via a linker. Suitable linkers include any chemical group that contains a primary amine and include amino acids, primary amine-containing alkyl, alkenyl or arenyl groups. The connection between the linker and the therapeutic drug may be of any type know in the art, preferably covalent bonding.

In certain embodiments, the linker comprises an amino acid or amino acid sequence. The sequence may be of any length, but is preferably between 1 and 10 amino acids, most preferably between 1 and 5 amino acids. Preferred amino acids are leucine or an amino acid sequence containing this amino acid, especially at their amino termini.

The prodrug compounds can be prepared according to standard synthetic or recombinant techniques known to those of skill in the art. For instance, peptide linking moieties can be synthesized by conventional solid phase or solution phase peptide chemistry. Biologically active entities and masking moieties can be obtained from commercial sources or from other well-known methods such as purification from natural sources, recombinant expression and other techniques. Dual polarity linkers and spacer moieties can be synthesized or obtained from commercial sources or from other well-known methods.

Typically, the prodrugs are prepared synthetically by condensing the masking moiety and biologically active entity with the linking moiety. Well known protecting groups can be used advantageously in the preparation of prodrug compounds. If the linking moiety is a peptide and the biologically active entity is a polypeptide and a terminus of the linking moiety is linked to a complementary terminus of the biologically active entity via an amide bond, the prodrug, or a portion thereof, can conveniently be prepared by recombinant synthesis. A nucleic acid coding for the amino acid sequence of the linking moiety and the biologically active agent can be prepared and used to express the covalent linking moiety-biologically active agent complex by standard techniques (see, e.g., Ausubel et al., 1987, Current Protocols in Molecular Biology, John Wiley & Sons, Inc., New York). The masking moiety can then be linked, for instance, to the amino terminus of the linking moiety by standard solution phase peptide chemistry. If the masking moiety is also a peptide or polypeptide and a terminus of the masking moiety is also linked to a complementary terminus of the linking moiety via an amide bond, the entire prodrug can conveniently be prepared by recombinant synthetic techniques. The nucleic acid expressing the prodrug should encode the amino acid sequences of the masking moiety, the linking moiety and the biologically active entity in tandem. Prodrugs produced by recombinant synthesis can be expressed in any eukaryotic or prokaryotic system in which the linking moiety is not cleaved by proteases, peptidases or other factors.

### REFERENCES

1. Ranson, M. & Jayson, G. Targeted antitumour therapy-future perspectives. Br J Cancer 92 Suppl 1, S28-31 (2005).
2. Wohnan, S. R. & Heppner, G. H. Genetic heterogeneity in breast cancer. J Natl Cancer Inst 84, 469-470 (1992).
3. Reya, T., Morrison, S. J., Clarke, M. F. & Weissman, I. L. Stem cells, cancer, and cancer stem cells. Nature 414, 105-111 (2001).
4. Lengauer. C., Kinzler, K. W. & Vogelstein, B. Genetic instabilities in human cancers. Nature 396, 643-649 (1998).
5. Fidler, I. J. The pathogenesis of cancer metastasis: the ‘seed and soil’ hypothesis revisited. Nat Rev Cancer 3, 453-458 (2003).
6. Kitano, H. Cancer robustness: tumour tactics. Nature 426, 125 (2003).
7. Mueller, M. M. & Fusenig, N. E. Friends or foes—bipolar effects of the tumour stroma in cancer. Nat Rev Cancer 4, 839-849 (2004).
8. Cheng, J. D. & Weiner, L. M. Tumors and their microeavironments: tilling the soil. Commentary re: A. M. Scott et al., A Phase I dose-escalation study of sibrotuzumab in patients with advanced or metastatic fibroblast activation protein-positive cancer. Clin. Cancer Res., 9: 1639-1647, 2003. Clin Cancer Res 9, 1590-1595 (2003).
9. Folkman, J. Tumor angiogenesis: therapeutic implications. N Engl J Med 285, 1182-1186 (1971).
10. Dvorak, H. F. Tumors: wounds that do not heal. Similarities between tumor stroma generation and wound healing. N Engl J Med 315, 1650-1659 (1986).
11. Tuxhorn, J. A. et al. Reactive stoma in human prostate cancer: induction of myofibroblast phenotype and extracellular matrix remodeling. Clin Cancer Res 8, 2912-2923 (2002).
12. Schuler, T., Kornig, S. & Blankenstein, T. Tumor rejection by modulation of tumor stromal fibroblasts. J Exp Med 198, 1487-1493 (2003).
13. Kelly, T. Fibroblast activation protein-alpha and dipeptidyl peptidase IV (CD26): cell-surface proteases that activate cell signaling and are potential targets for cancer therapy. Drug Resist Updat 8, 51-58 (2005).
14. Rettig, W. J. et al. Cell-surface glycoproteins of human sarcomas: differential expression in normal and malignant tissues and culted cells. Proc Natl Acad Sci USA 85, 3110-3114 (1988).
15. Garin-Chesa, P., Old, L. J. & Rettig, W. J. Cell surface glycoprotein of reactive stromal fibroblasts as a potential antidody target in human epithelial cancers. Proc Natl Acad Sci USA 87, 7235-7239 (1990).
16. Welt, S. et al. Antibody targeting in metastatic colon cancer: a phase I study of monoclonal antibody F19 against a cell-surface protein of reactive tumor stromal fibroblasts. J Clin Oncol 12, 1193-1203 (1994).
17. Aertgeerts, K. et al. Structural and kinetic analysis of the substrate specificity of human fibroblast activation protein alpha. J Biol Chem 280, 19441-19444 (2005).
18. Park, J. E. et al. Fibroblast activation protein, a dual specificity serine protease expressed in reactive human tumor stromal fibroblasts. J Biol Chem 274, 36505-36512 (1999).
19. Pineiro-Sanchez, M. L. et al. Identification of the 170-kDa melanoma membrane-bound gelatinase (seprase) as a serine integral membrane protease. J Biol Chem 272, 7595-7601 (1997).
20. Tahtis, K. et al. Expression and targeting of human fibroblast activation protein in a human skin/severe combined immunodeficient mouse breast cancer xenograft model. Mol Cancer Ther 2, 729-737 (2003).
21. Cheng, J. D. et al. Promotion of tumor growth by murine fibroblast activation protein, a seine protease, in an animal model. Cancer Res 62, 4767-4772 (2002).
22. Ramirez-Montagut, T. et al. FAPalpha, a surface peptidase expressed during wound healing, is a tumor suppressor. Oncogene 23, 5435-5446 (2004).
23. Hofheinz, R. D. et al. Stromal antigen targeting by a humanised monoclonal antibody: an early phase II trial of sibrotuzumab in patients with metastatic colorectal cancer. Onkologie 26, 44-48 (2003).
24. Mersmann, M. et al. Human antibody derivatives against the fibroblast activation protein for tumor stroma targeting of carcinomas. Int J Cancer 92, 240-248 (2001).
25. Schmidt, A. et al. Generation of human high-affinity antibodies specific for the fibroblast activation protein by guided selection. Eur J Biochem 268, 1730-1738 (2001).
26. Scott, A. M. et al. A Phase I dose-escalation study of sibrotuzumab in patients with advanced or metastatic fibroblast activation protein-positive cancer. Clin Cancer Res 9, 1639-1647 (2003).
27. Tanswell, P. et al. Population pharmacokinetics of antifibroblast activation protein monoclonal antibody F19 in cancer patients. Br J Clin Pharmacol 51, 177-180 (2001).
28. Wuest, T., Moosmayer, D. & Pfizenmaier, K. Construction of a bispecific single chain antibody for recruitment of cytotoxic T cells to the tumour stroma associated antigen fibroblast activation proton J Biotechnol 92, 159-168 (2001).
29. Adams, S. et al PT-100, a small molecule dipeptidyl peptidase inhibitor, has potent antitumor effects and augments antibody-mediated cytotoxicity via a novel immune mechanism. Cancer Res 64, 5471-5480 (2004).
30. Cheng, J. D. et al. Abrogation of fibroblast activation protein enzymatic activity attenuates tumor growth. Mol Cancer Ther 4, 351-360 (2005).
31. Mhaka, A. et al. Use of methotrexate-based peptide substrates to characterize the substrate specificity of prostate-specific membrane antigen (PSMA). Cancer Biol Ther 3, 551-558 (2004).
32. de Groot, F. M. et al. Design, synthesis, and biological evaluation of a dual tumor-specific motive containing integrin-targeted plasmin-cleavable doxorubicin prodrug. Mol Cancer Ther 1, 901-911 (2002).
33. DeFeo-Jones, D. et al. A prostate-specific antigen (PSA)-activated vinblastine prodrug selectively kills PSA-secreting cells in vivo. Mol Cancer Ther 1, 451-459 (2002).
34. DeFeo-Jones, D. et al. A peptide-oxorubicin ‘prodrug’ activated by prostate-specific antigen selectively kills prostate tumor cells positive for prostate-specific antigen in vivo. Nat Med 6, 1248-1252 (2000).
35. Pan, C. et al. CD10 is a key enzyme involved in the activation of tumor-activated peptide prodrug CPI-0004Na and novel analogues: implications for the design of novel peptide prodrugs for the therapy of CD10+ tumors. Cancer Res 63, 5526-5531 (2003).
36. Dubois, V. et al. CPI-0004Na, a new extracellularly tumor-activated prodrug of doxorubicin: in vivo toxicity, activity, and tissue distribution confirm tumor cell selectivity. Cancer Res 62, 2327-2331 (2002).
37. Albright, C. F. et al. Matrix metalloproteinase-activated doxorubicin prodrugs inhibit HT1080 xenograft growth better than doxorubicin with less toxicity. Mol Cancer Ther 4, 751-760 (2005).
38. Mansour, A. M. et al. A new approach for the treatment of malignant melanoma:

enhanced antitumor efficacy of an albumin-binding doxorubicin prodrug that is cleaved by matrix metalloproteinase 2. Cancer Res 63, 4062-4066 (2003).
39. Demneade, S. R. et al. Prostate-specific antigen-activated thapsigargin prodrug as targeted therapy for prostate cancer. J Natl Cancer Inst 95, 990-1000 (2003).
40. Barinka, C. et al. Substrate specificity, inhibition and enzyimological analysis of recombinant human glutamate carboxypeptidase II. J Neurochem 80, 477-487 (2002).
41. Sun, S. et al. Expression, purification, and kinetic characterization of full-length human fibroblast activation protein. Protein Expr Purif 24, 274-281 (2002).
42. Vihinen, P., Ala-aho, R. & Kahari, V. M. Matrix metalloproteinases as theapeutic targets in cancer. Curr Cancer Drug Targets 5, 203-220 (2005).
43. Maggiora, L. L., Smith, C. W. & Zhang, Z. Y. A general method for the preparation of internally quenched fluorogenic protease substrates using solid-phase peptide synthesis. J Med Chem 35, 3727-3730 (1992).
44. Bulleid, N. J., John, D. C. & Kadler, K. E. Recombinant expression systems for the production of collagen. Biochem Soc Trans 28, 350-353 (2000).
45. Olsen, D. et al. Expression and characterization of a low molecular weight recombinant human gelatin: development of a substitute for animal-derived gelatin with superior features. Protein Expr Purif 40, 346-357 (2005).
46. Backes, B. J., Harris, J. L., Leonetti, F., Craik, C. S. & Ellman, J. A. Synthesis of positional-scanning libraries of fluorogenic peptide substrates to define the extended substrate specificity of plasmin and thrombin. Nat Biotechnol 18, 187-193 (2000).
47. Janssen, S. et al. Screening a combinatorial peptide library to develop a human glandular kallikrein 2-activated prodrug as targeted therapy for prostate cancer. Mol Cancer Ther 3, 1439-1450 (2004).
48. Deng, S. J. et al. Substrate specificity of human collagenase 3 assessed using a phage-displayed peptide library. J Biol Chem 275, 31422-31427 (2000).
49. Matthews, D. J. & Wells, J. A. Substrate phage: selection of protease substrates by monovalent phage display. Science 260, 1113-1117 (1993).
50. Knauper, V., Lopez-Otin, C., Smith, B., Knight, G. & Murphy, G. Biochemical characterization of human collagenase-3. J Biol Chem 271, 1544-1550 (1996).
51. McGeehan, G. M. et al. Characterization of the peptide substrate specificities of interstitial collagenase and 92-kDa gelatinase. Implications for substrate optimization. J Biol Chem 269, 32814-32820 (1994).
52. Matsushita, O., Yoshihara, K, Katayama, S., Minwi, J. & Okabe, A. Purification and characterization of Clostridium perfringens 120-kilodalton collagenase and nucleotide sequence of the corresponding gene. J Bacteriol 176, 149-156 (1994).
53. Hori, H. & Nagai, Y. Purification of tadpole collagenase and characterization using collagen and synthetic substrates. Biochim Biophys Acta 566, 211-221 (1979).
54. Tsu, C. A., Perona, J. J., Schellenberger, V., Turck, C. W. & Craik, C. S. The substrate specificity of Uca pugilator collagenolytic serine protease 1 correlates with the bovine type I collagen cleavage sites. J Biol Chem 269, 19565-19572 (1994).
55. Patterson, S. D. & Aebersold, R. Mass spectrometric approaches for the identification of gel-separated proteins. Electrophoresis 16, 1791-1814 (1995).
56. Firestone, e.a. FAP-activated anti-tumour compounds (U.S. Pat. No. 6,613,879). USPTO (2003).

All patents, patent applications, references and other documents identified herein are incorporated in their entirety herein by reference.

### EXAMPLES

It should be appreciated that the invention should not be construed to be limited to the examples now described; rather, the invention should be construed to include any and all applications provided herein and all equivalent variations within the skill of the ordinary artisan.

Materials—The Drosophila Expression System (DES)⁵⁰ was from Invitrogen Rockville, Md.). Peptide Ala-Pro-AFC was from Bachem (Heidelberg, Germany). Gly-Pro-AMC, MMP substrate sampler kit and all other peptide synthesis reagents were from Anaspec (San Jose, Calif.). Novatag Dnp resin, Mca-Osu, HOBt, NMP were from Novabiochem, San Diego, Calif. Unless otherwise indicated all the other reagents were from Sigma-Aldrich (St. Louis, Mo.).

FAP Cloning and Epression—A PCR approach was used to amplify and attach a His-6 tag to the amino terminus of the extra-cellular domain of FAP (Genbank accession number NM_{—}004460). Primers used were (forwardBg1II) 5′ GGAAGATCTCATCATCACCATCACCATCGCCCTTCAAG 3′ and (reverseXhoI) 5′ GGCCTCGAGTCATTAGTCTGACAAAGAGAAACACTGC 3′. Template amplification was performed using Pfu-polymerase (Promega, Madison) as per suggested protocol. A PCR reaction began with an initial denaturation stp (94° C. for 2 mins) followed by 3 cycles of amplification (94° C. for 30 s, 40° C. for 1 min, 72° C. for 2 mins), followed by 30 cycles of amplification (94° C. for 30 s, 58° C. for 1 min, 72° C. for 2 mins), and ended with a final extension step (72° C. for 10 mins). A 2 kb PCR fragment was purified by gel electrophoresis, digested with Bg1II/XhoI and cloned into pMT/BiP/V5-HisA (Invitrogen, Calif.) previously digested with same set of enzymes. Final construct was designated as pMT-His-FAP.

Transfection of insect cells and stable cell line generation—Schneider's S2 cells (invitrogen) were maintained in Drosophila Expression System (DES) medium (Gibco, Rockville, Md.) supplemented with 10% heat inactivated fetal bovine serum (FBS) at room temperature. Before transfection, the cells were seeded in a 35-mm dish and grown until they reached a density of 2-4 10⁶ cells/mL. The cells were cotransfected with 19 μg of pMT-His-FAP and 1 μg of a pCoHYGRO selection vector using a kit for calcium phosphate-mediated transfection (Invitrogen). The calcium phosphate solution was removed 16 h post-transfection and fresh DES medium supplemented with 10% FBS was added (a complete medium). The cells were grown for additional 2 days and then the medium was replaced with the complete medium containing 400 μg/NL HygromycinB (Invitrogen). The selection medium was changed every 3-4 days. Extensive cell death of non-transfected cells was evident after about 1 week and cells resistant to Hygromycin B started to grow out 2-3 weeks post-transfection.

His tagged FAP large scale expression and purification—The hygromycin-resistant cells were seeded in 10 T-150s at a density of 1 million/ml. When cells reached density of 2-3 million/ml, 500 μMCuSO₄ was used to induce FAP expression. Cells were grown until they reached a density of 10-15 million cells/ml (8-9 days). 2 ml of 200 mM L-glutamine was added to the cell suspension on days 2 and 6.

Conditioned media containing secreted FAP collected after 12-14 days, cells and debris were removed by centrifugation at 4000 g for 30 mins, followed by filtering through 0.22 μm pore size filter. Media was concentrated and excess CuSO₄ was removed by 3 rounds of ultrafiltration using Amicon 8480 membrane (Millipore) with 30,000 Kda cutoff. After each round of ultrafiltration, volume was made up using sterile water. Final purification was obtained by incubating the concentrate with Ni—NTA resin (Qiagen, Calif.) in manufacturer recommended salt and imidazole concentration. FAP was eluted from resin using 250 mM imidazole. Final 30 ml of eluate was diluted with water to 300 ml and imidazole was removed by 2 rounds of ultrafiltration. Purity was checked by SDS-PAGE Coomassie staining. Western Blot was probed with Anti-His tag [Penta-His-Horse Radish Peroxidase (HRP) Conjugate from Qiagen, Calif.). Overall, a yield of 1-2 mgs was obtained from a 700 ml culture. Final purified aliquots were stored in reaction buffer at −20° C.

FAP Dipeptidyl Peptidase Assay—Quantitative assay for dipeptidyl peptidase activity were developed using Ala-Pro-AFC as the substrate as described by Park et al¹⁸. Purified protein was mixed with 5-10-fold volumes of reaction buffer (100 mM NaCl, 100 mM Tris, pH 7.8), and added to an equal volume of 0.5 mM Ala-Pro-AFC in reaction buffer followed by incubation for 1 h at 37° C. Release of free AFC was measured in a DTX 880 Multimode Detector (Beckman, Fullerton, Calif.) using the 395 mn excitation/530 nm emission filter set.

FAP Gelatinase and Collagenase Assay—Quenched gelatin and collagen conjugates were used to detect and confirm FAP's gelatinase and collagenase activity. DQ™ Gelatin from pig skin, DQ™ collagen, type IV from human placenta, DQ™ collagen, type I from bovine skin, all as fluorescent conjugates (Invitrogen, Rockville, Md.) were digested with FAP and digestion monitored on a fluorescence plate reader. Protein substrates were dissolved in reaction buffer (100 mM NaCl, 100 mM Tris, pH 7.8) to a final concentration of 100 μgs/ml. Trypsin digestion was used as a positive control. As a negative control the His tagged extra cellular domain of Prostate Specific Membrane Antigen (PSMA), which was similarly purified from S2 cells under the same conditions as FAP. Fluorescent quenched DQ™ Bovine Serum Albumin was used as a negative control for FAP protease activity.

Profiling FAP substrate specificity with substrates for Matrix Metallo-Proteases (MMPs)—16 Fluorogenic MMP substrates were obtained from Anaspec (Calif.) as a EnzoLyte™ 520 MMP Substrate Sampler Kit. The proteolytic cleavage of fluorescence quenched substrates was monitored at 485/535 nm on a plate reader. Substrates are supplied in DMSO at a concentration of 100 uM. To reduce inhibition of FAP enzyme activity because of DMSO, all substrates were diluted 10 fold in reaction buffer to a final concentration of 10 uM. A high concentration of FAP was also used to compensate for inhibition by 10% DMSO. Controls were just the substrates either with BSA or buffer. Cleaved substrates were purified and prepared for Mass Spectrometry analysis as described below.

Digestion of human collagen I and recombinant gelatin with FAP for cleavage mapping—Human collagen I (Becton Dickinson, Franklin Lakes, N.J.) and recombinant human gelatins of 100 KDa and 8.5 KDa (Fibrogen, San Francisco, Calif.) were dissolved in reaction buffer to a final concentration of 100-300 μg/ml. 0.5-1 μg of FAP was added per 100 μgs of protein substrate. Digestion was done for 4-6 hrs at 37° C. As a positive control trypsin digestion was used. As a negative control, protein solutions were incubated either with BSA or buffer only. Peptide fragments of a size <30 KDa were purified using 30 KDa Microcon spin filter (Millipore, Billerica, Mass.). The fragments were further purified with C₁₈ spin tubes (Agilent, Palo Alto, Calif.) as per suggested protocol with the substitution of 0.5% Acetonitrile in place of 5% for binding and washing of the C₁₈ columnus. Samples were prepared for Matrix Assisted Laser Desorption/Ionization-Time of Flight (MALDI-TOF) analysis by dilution with 2,5-dihydroxybenzoic acid (DHB) as the matrix.

Nano-flow HPLC and Mass Spectrometry-Peptides obtained from FAP/gelatin digests were dried using a speedvac (Eppendorf), resuspended in LC/MS loading buffer (3% ACN, 0.1% formic acid), and analyzed using nano-flow LC/MS/MS on an Agilent 1100 series nano-LC system (Agilent) coupled to an LCQ Duo ion trap mass spectrometer (ThermoFinnigan). Peptides were pre-concentrated on a 5 mm Zorbax C18 trap column (Agilent) and then eluted onto a 100×0.075 mm custom-packed Biobasic C18 (ThermoElectron) reversed phase capillary column connected to a laser-pulled electrospray ionization emitter tip (New Objective) at a flowrate of 300 nl/min. Peptides were elated the nanospray source of the LCQ (Proxeon, Denmark) using the following gradient 0% B at 0 min, 5% B at 8 min, 45% B at 50 min, 90% B at 55 min, 90% B at 60 min (B=0.1% formic acid in acetonitrile) at a spray voltage of 2.5 kV. The LCQ was operated in data-dependent mode using the Xcalibur software (ThermoFinnigan) in which every MS scan (400-1800 m/z) was followed by MS/MS scans (400-1800 m/z) on the 3 most intense ions using an isolation window of ±1.5 Da. Ions selected for MS/MS fragmnentation were dynamically excluded for 30 s.

MS/MS data were searched against a collagen FASTA database using the SEQUEST search algorithm built into the Bioworks Browser (ThermoFinnigan) allowing for the variable modification of Methionine oxidation. Peptides were initially filtered in a charge dependent manner using an XCorr filter of 1.5, 2, and 2.5 for singly, doubly, and triply charged peptides. All MS/MS spectra used to identify peptides were manually inspected for validation of y and b-ion series. To quantify the relative abundance of each identified peptide we compared the ion current for each of the observed peptide parent ions firm the MS spectra. The contribution of each parent ion to the total ion current was extracted and integrated over the peptide elution peak

### FAP Digest of Recombinant Human Gelatins

As Collagen I and Gelatin are currently the only known protein substrates for FAP, we needed to develop an alternative method to identify FAP-selective cleavage sites within these proteins. To solve the problem of post-translational modification we identified a source of recombinant human gelatin and collagen from FibroGen (South San Francisco, Calif.) that have been prepared by cloning human Collagen I sequence in a strain of Pichia pastoris which lacks the enzyme Prolyl Hydroxylase (51). These gelatins are well characterized and have no post translational modifications. The gelatins are used to produce drug capsules and as vaccine adjuvants. Therefore, using these recombinant proteins, the above mentioned protocol for MALDI sample preparation and analysis was used to analyze FAP digestion of an 8.5 kDa fragment of this recombinant form of human gelatin. SDS PAGE and MALDI spectra showed that FAP digests the recombinant gelatin. The masses of fragments <3 KDa were purified for MALDI spectra and again analyzed by FindPept tool. However, once again multiple peptide sequences were obtained for each cleavage fragment (data not shown). Therefore, additional methodology had to be developed to resolve each particular mass fragment using Liquid Chromatograpy (LC) and Tandem mass spectroscopy (MS MS). An LC-MS-MS method was developed. Using this method, peptides obtained from the FAP gelatin digests were dried using a speedvac (Eppendorf), resuspended in LC/MS loading buffer (3% ACN, 0.1% formic acid), and analyzed using nano-flow LC/MS/MS on an Agilent 1100 series nano-LC system (Agilent) coupled to an LCQ Duo ion trap mass spectrometer (ThermoFinnigan). Peptides were eluted the nanospray source of the LCQ (Proxeon, Denmark) using the following gradient: 0% B at 0 min, 5% B at 8 min, 45% B at 50 min, 90% B at 55 min, 90% B at 60 min (B=0.1% formic acid in acetonitrile) at a spray voltage of 2.5 kV. The LCQ was operated in data-dependent mode using the Xcalibur software (ThermoFinnigan) in which every MS scan (400-1800 m/z) was followed by MS/MS scans (400-1800 m/z) on the 3 most intense ions using an isolation window of ±1.5 Da. Ions selected for MS/MS fragmentation were dynamically excluded for 30 s.

MS/MS data were searched against a collagen FASTA database using the SEQUEST search algorithm built into the Bioworks Browser (ThermoFinnigan) allowing for the variable modification of Methionine oxidation. Peptides were initially filtered in a charge dependent manner using an XCorr filter of 1.5, 2, and 2.5 for singly, doubly, and triply charged peptides. All MS/MS spectra used to identify peptides were manually inspected for validation of y and b-ion series. To quantify the relative abundance of each identified peptide we compared the ion current for each of the observed peptide parent ions from the MS spectra. In an attempt to identify the more preferred FAP cleavage sites within the 8.5 kDa fragment, the contribution of each parent ion to the total ion current was extracted and integrated over the peptide elution peak. These ion currents were normalized such that peak with lowest ion current was assigned a value of 1.0, Table 3.

TABLE 3
FAP cleavage sites (P7-P′3) within the 8.5 kDa
fragment of recombinant human gelatin

| | Cleavage | | | Normalized |
|---|---|---|---|---|
| | Fragment | | MH+ | Ion Current |
| PPGAVGP/ | AGK . . . AQGPPGP/ | AGP | 1308.62 | 234.8 |
| — | GLP . . . SPGSPGP/ | DGK | 1449.77 | 76.3 |
| KTGPPGP/ | AGQ . . . PPGPPGA/ | RGQ | 1330.65 | 57.2 |
| VMGFPGP/ | KGA . . . PPGAVGP/ | AGK | 2113.12 | 46.8 |
| VMGFPGP/ | KGA . . . GEPGKAG/ | ERG | 942.5 | 14.7 |
| — | GLP . . . KTGPPGP/ | AGQ | 2256.16 | 12.7 |
| GFPGPKG/ | AAG . . . PPGAVGP/ | AGK | 1928 | 10.1 |
| FPGPKGA/ | AGE . . . PPGAVGP/ | AGK | 1856.96 | 6.6 |
| LTGSPGS/ | PGP . . . KTGPPGP/ | AGQ | 1076.54 | 6.0 |
| KTGPPGP/ | AGQ . . . GPPGPPG/ | ARG | 1259.61 | 5.0 |
| VMGFPGP/ | KGA . . . AGEPGKA/ | GER | 885.48 | 4.7 |
| GLPGAKG/ | LTG . . . SPGSPGP/ | DGK | 869.44 | 2.7 |
| MGFPGPK/ | GA- AGEPGKA/ | GER | 757.38 | 2.5 |
| PGPPGAR/ | GQA . . . VMGFPGP | KGA | 1017.48 | 2.4 |
| PGARGQA/ | G- VMGFPGP/ | KGA | 761.37 | 1.7 |
| GPPGPPG/ | ARG . . . VMGFPGP/ | KGA | 1244.62 | 1.7 |
| PPGPPGA/ | RGQ . . . VMGFPGP/ | KGA | 1173.58 | 1.3 |
| KTGPPGP/ | AGQ . . . GARGQAG/ | VMG | 1799.89 | 1.0 |

Analysis of data in Table 3 demonstrate that FAP cleavage primarily occurred after proline (P), but FAP could also cleave after other amino acids including glycine (G), alanine (A), lysine (K) and arginine (R) (underlined sequences in Table 3). Based on normalized ion current, it appeared that more abundant ions consisted of those with Proline as cleavage site. In those sequences, G was the preferred amino acid in the P2 position.

Synthesis of substrates based on determined cleavage sites—Quenched substrates were prepared by using the MCA/DNP fluorophore/quencher pair. Synthesis of peptides was done using standard Fmoc solid phase coupling on NovaTag™ Dnp resin with a substitution level of 0.4 mmole/g (Novabiochem, San Diego, Calif.). N-terminal capping was done twice overnight with N-(7-methoxycoumarin-4-acetyloxy)succinimide (MCA-Osu) and 1-Hydroxybenzotriazole (HOBt) in N-Methyl 2 Pyrrolidone (NMP). Peptides were cleaved with 95% TFA, 2.5% TIS and 2.5% water. The purity and mass of each quenched peptide was confinned by Reversed Phase-HPLC and MALDI-TOF analysis.

Comparative analysis of FAP hydrolyses of quenched substrates—Quenched peptide substrates were weighed and dissolved in DMSO to obtain a final concentration of 50 mM and were stored at −20° C. until later use. Dilutions (e.g. 200 μM, 100 μM, 50 μM, 25 μM) were prepared in duplicates in the reaction buffer. His-tagged FAP was added to a final concentration of 5 nM. Release of free MCA was measured at excitation 355 nm and emission 460 nm in a 96-well fluorescence plate reader. Controls consisted of substrates in buffer±BSA.

### Hydrolysis of FAP Fluorescence Quenched Substrates

On the basis of the 8.5 kDa gelatin cleavage map a series of fluorescence quenched substrates were prepared (colored sequences in Table 3) by using the Methoxycoumarin (MCA)/Dinitrophenyl (DNP) FRET combination (52). Synthesis of peptides was done using standard Fmoc solid phase peptide synthesis coupling on a NovaTag™ Dnp resin with a substitution level of 0.4 mmole/g (Novabiochem, San Diego, Calif.). N-terminal capping was done twice overnight with N-(7-methoxycoumarin-4-acetyloxy)succinimide (MCA-Osu) and 1-Hydroxybenzotriazole (HOBt) in N-Methyl 2 Pyrrolidone (NMP). This synthetic method yields peptides with sequence MCA-AA1-AA2-AA3-AAx-DNP. These studies supported substrate ranking based on normalized ion current and confirmed that FAP prefers to cleave after proline but can also cleave after other amino acids in the P1 position, FIG. 10A. The results also suggest that FAP hydrolysis increases with increasing number of amino acids in the P′ positions with VGP//AGK cleavage>GAVGP//A>PAGP//, FIG. 10. Kinetic analysis performed on the best two substrates from this initial substrate screen demonstrated Kₘ values <100 μM that are lower than those reported for fluorescent dipeptide substrates AP-AMC and Z-GP-AMC, FIG. 10B.

In reference to FIG. 12, (A) shows FAP Hydrolysis rates of fluorescently quenched peptide with indicated peptide sequences assayed at concentration of 30 μM. Relative change in fluorescence measured in 96 well fluorescent plate reader (Fluoroscan II). FIG. 12(B) shows Michaelis Menten plots of PGP//AGQ and VGP//AGK with kinetic parameters calculated using Enzyme Kinetics Module from Sigma Plot 8.0 software.

The characterization of protease substrate specificity requires that the protease be pure and correctly folded to maintain enzymatic activity. Previously it had been shown that full length FAP, cloned and expressed in Drosophila S2 cells, yielded highly pure protein that was enzymatically similar to the human form⁴¹. Therefore, the extracellular domain of FAP was cloned with a His-6 tag at its N-terminus to generate a stable FAP-producing Drosophila S2 cell line. On induction with CuSO₄ FAP was secreted into the media which was conditioned and then concentrated by ultra filtration and purified using Ni—NTA beads. Purified FAP was demonstrated to be enzymatically active via its ability to cleave the dipeptide substrate Ala-Pro-AFC with the same kinetic parameters as previously described (REF). Coomassie stain and western blot analysis with an Anti-His tag mAb documented the correct protein size of ˜80 KDa.

Quenched forms of Gelatin and Collagen were used to confirm the gelatinase and collagenase activity of recombinant FAP. Quenching is achieved by labeling these proteins with the fluorophore FITC such that fluorescence signal from the intact protein is minimal due to is self-quenching by the fluorphore. Protein digestion releases FITC-labeled fragments that result in measurable increase in overall fluorescence signal from the reaction mixture. Previously, it had been demonstrated using gel zymography that FAP can cleave gelatin and collagen I but could not cleave collagen IV. To confirm these results with our recombinant His-tagged FAP we used the FITC-quenched proteins DQ™ Gelatin from pig skin, DQ™ collagen IV from human placenta, DQ™ collagen I from bovine skin and DQ™ BSA as a control. In this assay, Gelatin and Collagen I were readily hydrolyzed by FAP while Collagen IV and BSA (data not shown) were not. On a relative basis gelatin was digested ˜10-fold better by FAP than collagen I. MALDI analysis of digested fragments was performed to confirm hydrolysis. As a negative control, we demonstrated no digestion of any of the proteins using purified His-tagged human carboxypeptidase PSMA from Drosophila S2 cells under the same conditions. These results confirm that Gelatin and Collagen I hydrolysis was due to FAP and not due to the presence of some other protease contaminating our purification system.

### MALDI for FAP Digest of Human Collagen I

In an effort to elucidate the substrate specificity of FAP, we examined FAP digests of unlabeled human Collagen I using matrix assisted laser desorption ionization (MALDI) time of flight mass spectrometry. Digestion reactions were performed at a substrate to protease mass ratio of 200:1 using recombinant FAP or modified trypsin as a control. Two negative controls of Collagen alone and FAP alone were also included to identify any peptides due to autolysis/degradation of these proteins. SDS-PAGE analysis of FAP digested Collagen I (not shown) produced a smear of continuous size fragments suggesting the presence of many cleavage sites. To simplify cleavage mapping by MALDI, small fragments (<3 kDa) were isolated by ultra filtration and further purified using reversed-phase chromatography. MALDI was subsequently performed using serial dilutions of the isolated peptides spotted with DHB matrix.

Masses of singly charged ions (MH+) obtained from MALDI spectra were entered into the Findpept search tool at the Expasy Proteomics Server (http://www.expasy.org/tools/findpept.html) and used to perform a peptide mass fingerprint (PMF) search against the known collagen sequence. MALDI spectra suggest that human Collagen I is cleaved by FAP at numerous specific sites (Table 1). In most cases, multiple peptide sequences were matched for the same mass and in some instances more than 30 sequences were obtained for one particular mass. This result was most likely due to the fact that human collagen I is a heterotrimeric polymer made up of repeating sequences containing the (GXY)n motif (where X=Pro, Y=HydroxyPro). Human collagen I is also known to be glycosylated, and cross-linked randomly throughout its sequence (65). These post-translational modifications in human collagen have not been well characterized and, therefore, make it more difficult to determine the exact cleavage sites using MALDI or other proteomics tools. These results, while confirming that FAP cleaves human collagen I, demonstrate the difficulties in obtaining correct cleavage sequences by mass spectroscopy due to the use of this poorly defined human collagen I

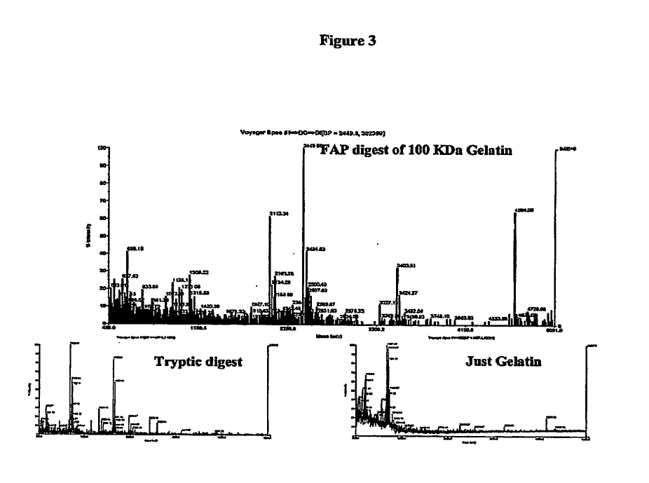

As Collagen I and Gelatin are currently the only known protein substrates for FAP, we developed an alternative method to identify FAP-selective cleavage sites within these proteins. To solve the problem of post-translational modification we identified a source of recombinant human gelatin and collagen from FibroGen (South San Francisco, Calif.) that have been prepared by cloning human Collagen I sequence in a strain of Pichia pastoris which lacks the enzyme Prolyl Hydroxylase (66). This human collagen I based gelatins is well characterized and has no post translational modifications. Therefore, using these recombinant proteins, the above mentioned protocol for MALDI sample preparation and analysis was used to analyze FAP digestion of the full size 100 kDa recombinant form of human gelatin and an 8.5 kDa fragment SDS PAGE separation and MALDI spectra showed that FAP readily digests the recombinant gelatins (FIG. 3 shows the spectra for 8.5 KDa Gelatin). The masses of fragments <3 KDa were purified for MALDI spectra and again analyzed using the FindPept tool. However, once again multiple peptide sequences were obtained for each cleavage fragment (Table 1).

### LC and Tandem MS/MS for FAP Digest of 8.5 KDa Gelatin Reveals the Cleavage Map

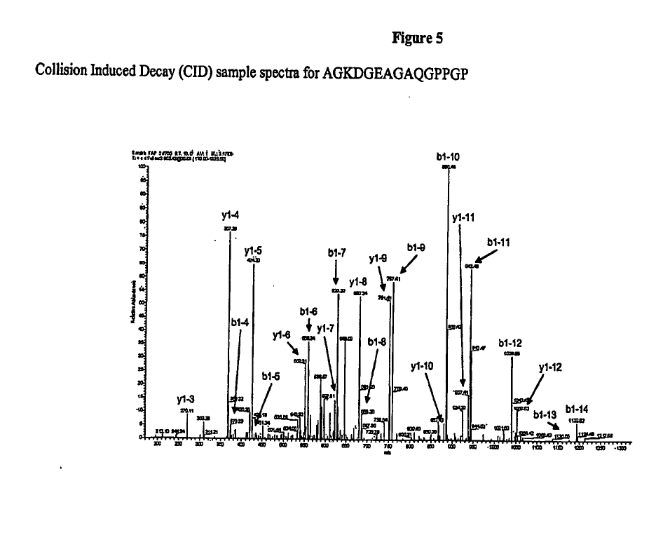

To resolve each particular mass fragment a Liquid Chromatograpy (LC) and Tandem mass spectroscopy (MS-MS) [LC-MS-MS] method was developed. Using this method, peptide fragments from FAP digests of the 8.5 kDa and 100 kDa recombinant gelatin were time-resolved by nano reverse phase LC (FIGS. 12 and 14) and then sequenced using an ion trap mass spectrometer operating in MS/MS mode. Initial methodological issues were worked out using the 8.5 kDa fragment before analyzing the entire 100 kDa protein. MS/MS spectra were searched against the 8.5 kDa and 100 kDa gelatin sequences using the SEQUEST algorithm with no cleavage specificity. Most of the identified cleavage sites in both sized gelatins occurred after Proline. As an example of MS/MS collision induced decay, the spectrum for P/AGKDGEAGAQGPPGP/A is shown in FIG. 5. However, FAP cleavage sites in these gelatins were not restricted to Proline alone. FAP was also found to cleave after Ala (e.g. A/A, A/G, A/P, A/R), Asp (e.g. D/G, D/T), Gly (e.g. G/A, G/E, G/L, G/Q, G/P, G/V), Glu (i.e., F/P), Lys (i.e., K/A, K/G), Ser (e.g. S/P) and Val (e.g. V/G).

### Mapping FAP Cleavage Sites in Human Collagen I

Previously it had been demonstrated using gel zymography that FAP could cleave Collagen I but not Collagen IV or fibronectin (18). However, other than dipeptide substrates, no other FAP substrate has been described. Therefore, we attempted to map FAP cleavage sites using unlabeled human Collagen I. For digestion a large ratio of 200:1 for substrate to protease was used. 100-200 μgs of human Collagen I were digested with 0.5-1 μgs of FAP. SDS-PAGE (not shown) revealed that the FAP digest of Collagen produced a smear of continuous size fragments. Absence of any distinct bands implied that peptide sequencing by Edman Degradation would not be possible. Therefore, mass Spectrometry was used to determine sequences of cleaved fragments. For this analysis Collagen and FAP alone were used as negative controls to identify any peaks due to autolysis/degradation of these proteins. As a positive control trypsin was used. To simplify mapping by MALDI, small size peptide fragments (<3 KDa) were isolated by ultra filtration using Microcon with 3 KDa cut off and further purified using C18 spin columns.

MALDI spectra revealed that human Collagen I was cleaved by FAP at certain specific sites. However, when masses (MH+) were put into the Findpept tool at Proteomics server Expasy (http://www.expasy.org/tools/findpept.html) multiple sequences were obtained for the same mass. In some cases more than 30 sequences were obtained for one particular mass. This result was most likely due to the fact that collagen I is a heterotrimeric polymer made up of repeating sequences containing the (GXY)n motif (X=Pro, Y=HydroxyPro). Human collagen I is also known to be glycosylated, and cross-linked (50). These post-translational modifications in human collagen make it even more difficult to determine exact cleavage sites by MALDI or other proteomics tools. These post-translational modifications have not been well characterized. These results, therefore, confirmed that FAP cleaves human collagen I but demonstrate the difficulties obtaining correct sequences by mass spec due to the use of poorly characterized human collagen I.

To identify the most preferentially cleaved sites, we relatively quantified the abundance of each of the identified peptides by integrating the ion current generated by each peptide throughout the chromatogram. After identification of a peptide from a MS/MS spectrum, we extracted the ion current for the parent mass of the ion from the total MS ion chromatogram using a 1.5 Da tolerance window, and then integrated under the peak (FIGS. 12 and 14). In the case of multiple peaks, we chose the peak nearest to the retention time of the MS/MS spectrum matched to the peptide sequence of interest. In the 8.5 kDa gelatin digest, P/AGKDGEAGAQGPPGP/A was the most abundantly identified fragment, whereas cleavage at the C-terminus of the protein generated the fragment P/VGPPGPPGPPGPPGPP as the most abundantly identified fragment in the 100 kDa gelatin digest.

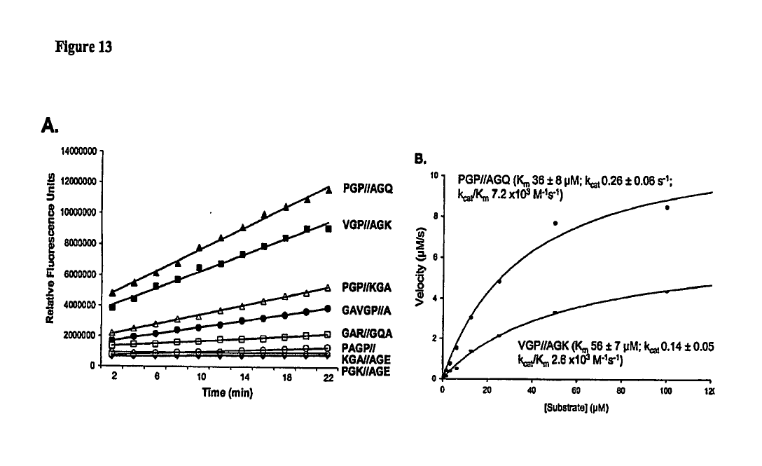

Three substrates were made using MCA/DNP fluorophore and quencher pair. Substrate PAGP has been previously used for prodrug design for FAP. Two of the new substrates made were VGPAGK and GARGQA, FIG. 13. All three substrates were hydrolyzed by FAP but for PAGP and GARGQA hydrolysis was seen only at concentration >200 μM. However, VGPAGK was rapidly hydrolyzed even at 25 μM.

We developed a method to identify substrates based on FAP's collagenase or gelatinase activity. Knowledge of substrate specificity was and can be used to elucidate FAP's biological role as well as for therapeutic targeting using prodrugs. Substrate specificity can be defined by either using high throughput methods like Positional Scanning Synthetic Combinatorial Library (PS-SCL)⁴⁶, One Bead One Peptide library⁴⁷ or by phage display^{48,49}. However these methods have the disadvantage of an artificial scaffold that can alter the physiological substrate specificity of a protease. Here we have described an approach to take the known protein substrates for a protease and map its cleavage site using proteomics. For the case of FAP, the problem was more complicated because of complex structure, sequence and many post translational modifications in collagen.

We generated a stable cell line of Drosophila S2 cells which secrets His tagged extra cellular domain of FAP. This allowed us to obtain highly pure FAP. It was confirmed to be active by its dipeptidase activity. It was further confirmed that it has gelatinase and collagenase activity by digesting quenched forms of these proteins. A specificity control of quenched BSA and collagen IV was not all digested by FAP. This implies that FAP's substrate specificity is more than its already known dipeptide substrates Ala-Pro-AFC, Lys-Pro-AFC and Gly-Pro-AFC. As already indicated, FAP's dipeptidase activity has a high Km of 500 μM-1 mM¹⁸. We wanted to find substrates that are better and specific than these dipeptides.

A large number of collagenases and gelatinases have been previously reported in literature. However for most of them, their substrate characterization was done using combinatorial libraries or synthetic model substrates^{48,50-53}. To our knowledge there is only one study in which Collagen was digested with a new type of Collagenase and sites were determined by Edman sequencing of specific bands isolated on SDSPAGE⁵⁴. However in that case only 10-12 cleavage fragments were obtained so, it was possible to easily separate them on SDS PAGE⁵⁴. In contrast, for FAP digest of collagen or gelatin, we saw more than 100 fragments, which looked like a smear. This implied that FAP is cleaving at many sites and sequencing by Edman might not be practical. So, we developed a mass spectrometry based approach to map these sites.

Human collagen I was digested with FAP and small size fragments (<3 KDa) were purified for MALDI-TOF analysis. Previously MADLI-TOF has been used to identify proteins by analysis of tryptic digests⁵⁵. Mass Spectra revealed that FAP was cleaving collagen I at specific sites. However, sites could not be identified by MALDI because each mass matched more than 10 sequences. An attempt was also made to do an LC tandem MS/MS on these fragments. However the data could not be solved because of collagen's several post translational modifications (PTMs) like hydroxylation, glycosylation and cross linking. Many of these PTMs have not been characterized so that meant human collagen cannot be easily used for such an analysis.

A recombinant gelatin form of human collagen I without any PTMs was used for cleavage mapping⁴⁵. The role of PTMs will remain to be investigated and it might be easier to study that based on data from recombinant gelatin. In this case gelatins of sizes 100 KDa and 8.5 KDa were used. MALDI spectra showed that both forms were hydrolyzed by FAP. The large sized gelatin again gave too many fragments that made mass spectrometry analysis difficult. So, the small sized gelatin digest was purified and analyzed by LC tandem MS/MS. The final map revealed many of the FAP cleavage sites, FIG. 12. Most of them are after Proline, though many novel cleavage sites like G/L, G/K, S/P, A/R, G/V, G/A, A/A, A/G, G/E were also found. This shows that the heterogeneity of collagen sequences can be exploited to extend the substrate specificity of a gelatinase or a collagenase. As a test eight sequences were synthesized as quenched substrates, FIG. 13. First was the sequence PAGP which has also been used by Bohringer Mannheim to design a prodrug for FAP⁵⁶. Other two sequences were VGPAGK and GARGQA. The hydrolysis of GARGQA confirmed that FAP can also cleave non proline based substrates. Though the concentration needed for GARGQA hydrolysis was >200 uM. The substrate PAGP also needed concentration >200 uM to detect any hydrolysis. In contrast, the substrate VGPAGK was rapidly hydrolyzed even at 25 uM. Other substrates based on cleavage map need to be tested and compared for their specificity and kinetic parameters.

Overall it was shown that this proteomics approach can be efficiently used to identify novel cleavage sites for FAP. This approach can also be easily used to map and extend the substrate specificity of large number of MMPs and other gelatinases which are already being targeted for designing drugs. Here we also showed that one of the test substrates based on cleavage mapping was more than 10-20 fold better than already known substrates. These substrates will be used as part of a prodrug or a protoxin.

TABLE 1

FindPept alignment of MALDI masses for digest of 8.5 KDa Gelatin

| | | | | | |
|---|---|---|---|---|---|
| | DB | Δmass | | | missed |
| User mass | mass | (daltons) | peptide | position | cleavages |

| | | | | | |
|---|---|---|---|---|---|
| 2114.530 | 2113.07 | −1.451 | (K)GAAGEPGKAGERGVPGPPGA VGPAG(K) | 55-79 | 0 |
| | 8 | | | | |
| 2114.530 | 2113.11 | −1.415 | (P)GPKGAAGEPGKAGERGVPGP PGAV(G) | 52-75 | 0 |
| | 5 | | | | |
| 2114.530 | 2113.11 | −1.415 | (G)PKGAAGEPGKAGERGVPGPP GAVG(P) | 53-76 | 0 |
| | 5 | | | | |
| 2114.530 | 2113.11 | −1.415 | (P)KGAAGEPGKAGERGVPGPPG AVGP(A) | 54-77 | 0 |
| | 5 | | | | |
| 2114.530 | 2113.11 | −1.415 | (A)AGEPGKAGERGVPGPPGAVG PAGK(D) | 57-80 | 0 |
| | 5 | | | | |
| 2114.530 | 2114.07 | −0.458 | (D)GRPGPPGPPGARGQAGVMGF PGP(K) | 31-53 | 0 |
| | 1 | | | | |
| 2114.530 | 2115.01 | 0.480 | (G)AVGPAGKDGEAGAQGPPGPA GPAGE(R) | 74-98 | 0 |
| | 0 | | | | |
| 2449.780 | 2448.24 | −1.534 | (Q)AGVMGFPGPKGAAGEPGKAG | 45-71 | 0 |
| | 5 | | ERGVPGP(P) | | |
| 2449.780 | 2451.16 | 1.384 | (T)GSPGSPGPDGKTGPPGPAGQ | 10-37 | 0 |
| | 5 | | DGRPGPPG(P) | | |
| 2449.780 | 2451.22 | 1.439 | (P)PGARGQAGVMGFPGPKGAAG | 39-65 | 0 |
| | 0 | | EPGKAGE(R) | | |
| 3402.320 | 3402.67 | 0.358 | (K)GAAGEPGKAGERGVPGPPGA | 55-94 | 0 |
| | 8 | | VGPAGKDGEAGAQGPPGPAG(P) | | |
| 3402.320 | 3402.71 | 0.394 | (F)PGPKGAAGEPGKAGERGVPG | 51-89 | 0 |
| | 5 | | PPGAVGPAGKDGEAGAQGP(P) | | |
| 3402.320 | 3402.71 | 0.394 | (P)GPKGAAGEPGKAGERGVPGP | 52-90 | 0 |
| | 5 | | PGAVGPAGKDGEAGAQGPP(G) | | |
| 3402.320 | 3402.71 | 0.394 | (G)PKGAAGEPGKAGERGVPGPP | 53-91 | 0 |
| | 5 | | GAVGPAGKDGEAGAQGPPG(P) | | |
| 3402.320 | 3402.71 | 0.394 | (P)KGAAGEPGKAGERGVPGPPG | 54-92 | 0 |
| | 5 | | AVGPAGKDGEAGAQGPPGP(A) | | |
| 3402.320 | 3403.67 | 1.351 | (T)GPPGPAGQDGRPGPPGPPGA | 22-59 | 0 |
| | 1 | | RGQAGVMGFPGPKGAAGE(P) | | |
| 3402.320 | 3403.67 | 1.351 | (P)PGPAGQDGRPGPPGPPGARG | 24-61 | 0 |

TABLE 2

Full map of FAP cleavage sites within 100 kDa human gelatinase

| | | | | | |
|---|---|---|---|---|---|
| P6-P2 | Sequence | P′2-P′4 | Mass | Occurences | Percentage |

| | | | | | |
|---|---|---|---|---|---|
| TGFPG | A.AGRVGPPGP.S | GNA | 807.448 | 2 | 0.95 |
| GPPGP | A.GPAGPPGP.I | GNV | 649.331 | 1 | 0.48 |
| GETGP | A.GPPGAPGAPGAPGP.V | GPA | 1099.554 | 1 | 0.48 |
| AGPPG | A.PGAPGAPGPVGPAGKSGDRGETGP.A | GPA | 2086.032 | 4 | 1.90 |
| PGAPG | A.PGAPGPVGPAGKSGDRGETGP.A | GPA | 1860.92 | 2 | 0.95 |
| PGPAG | A.PGDKGESGP.S | GPA | 843.385 | 2 | 0.95 |
| PGAPG | A.PGPVGPAGKSGDRGETGP.A | GPA | 1635.809 | 1 | 0.48 |
| GPPGA | D.GQPGAKGEPGDAGAKGDAGPPGP.A | GPA | 1987.947 | 2 | 0.95 |
| GPAGQ | D.GRPGPPGPPGARGQAG.V | MGF | 1428.746 | 2 | 0.95 |
| PGPSG | E.PGKQGPSGASGERGPPGP.M | GPP | 1632.809 | 5 | 2.38 |
| PAGFA | G.PPGADGQPGAKGEPGDAGAKGDAGPPGP.A | GPA | 2425.138 | 2 | 0.95 |
| ETGPA | G.PPGAPGAPGAPGPVGPAGKSGDRGETGP.A | GPA | 2408.196 | 7 | 3.33 |
| LTGPI | G.PPGPAGAPGDK.G | ESG | 963.49 | 2 | 0.95 |
| LTGPI | G.PPGPAGAPGDKGESGP.S | GPA | 1390.66 | 3 | 1.43 |
| AKGDA | G.PPGPAGPAGPPGP.I | GNV | 1068.548 | 1 | 0.48 |
| FPGLP | G.PSGEPGKQGPSGASGERGPPGP.M | GPP | 2002.958 | 1 | 0.48 |
| PSGPA | G.PTGARGAPGDRGEPGPPGP.A | GFA | 1742.857 | 6 | 2.86 |
| PGPPG | P.AGEKGSPGADGPAGAPGTPGP.Q | GIA | 1747.825 | 7 | 3.33 |
| PGPPG | P.AGFAGPPGADGQPGAKGEPGDAGAKGDAGPPGP.A | GPA | 2828.324 | 8 | 3.81 |
| PGAVG | P.AGKDGEAGAQGPPGP.A | GPA | 1308.618 | 2 | 0.95 |
| AGAAG | P.AGNPGADGQPGAKGANGAPGIAGAPGFPGARGP.S | GPQ | 2812.388 | 4 | 1.90 |
| KGDAG | P.AGPKGEPGSPGENGAPG.Q | MGP | 1478.688 | 1 | 0.48 |
| RGETG | P.AGPPGAPGAPGAPGP.V | GPA | 1170.591 | 8 | 3.81 |
| RGETG | P.AGPPGAPGAPGAPGPVGP.A | GKS | 1423.733 | 2 | 0.95 |
| RGETG | P.AGPPGAPGAPGAPGPVGPAGKSGDRGETGP.A | GPA | 2536.254 | 6 | 2.86 |
| QGLPG | P.AGPPGEAGKPGEQGVPGDLGAPGP.S | GAR | 2112.036 | 6 | 2.86 |
| PGPTG | P.AGPPGFPGAVGAKGEAGP.Q | GPR | 1536.781 | 2 | 0.95 |
| PGPTG | P.AGPPGFPGAVGAKGEAGPQGPRGSEGPQGVRGEPGPPGP.A | GAA | 3530.753 | 1 | 0.48 |
| PGPAG | P.AGPPGPIGNVGAPGAKGARGSAGPPGATGFPGAAGRVGPPGP.S | GNA | 3556.853 | 3 | 1.43 |
| SGPSG | P.AGPTGARGAPGDRGEPGPPGP.A | GFA | 1870.916 | 6 | 2.86 |
| TGPPG | P.AGQDGRPGPPGPPGARGQAG.V | MGF | 1799.89 | 1 | 0.48 |
| RGETG | P.AGRPGEVGPPGPPGP.A | GEK | 1341.691 | 11 | 5.24 |
| SGPQG | P.GGPPGPKGNSGEPGAPGSKGD.T | GAK | 1819.858 | 1 | 0.48 |
| GPRGL | P.GPPGAPGP.Q | GFQ | 649.331 | 1 | 0.48 |
| GRVGP | P.GPSGNAGPPGPP.G | PAG | 1004.48 | 1 | 0.48 |
| RGLTG | P.IGPPGPAGAPGDKGESGP.S | GPA | 1560.766 | 6 | 2.86 |
| MGFPG | P.KGAAGEPGKAGERGVPGPPGAVGP.A | GKD | 2113.115 | 5 | 2.38 |
| MGFPG | P.KGAAGEPGKAGERGVPGPPGAVGPAGKDGEAGAQGPPGP.A | GPA | 3402.715 | 1 | 0.48 |
| TGPAG | P.PGAPGAPGAPGPVGPAGKSGDRGETGP.A | GPA | 2311.143 | 2 | 0.95 |
| PGPMG | P.PGLAGPPGESGREGAPGAEGSPGRD.G | SPG | 2275.07 | 2 | 0.95 |
| TGPIG | P.PGPAGAPGDKGESGP.S | GPA | 1293.608 | 2 | 0.95 |
| PGAPG | P.QGFQGPPGEPGEPGASGP.M | GPR | 1685.751 | 2 | 0.95 |
| RGSEG | P.QGVRGEPGPPGP.A | GAA | 1147.585 | 3 | 1.43 |
| SGPAG | P.RGPPGSAGAPGKDGLNGLPGP.I | GPP | 1871.973 | 14 | 6.67 |
| PGLPG | P.SGEPGKQGPSGASGERGPPGP.M | GPP | 1905.906 | 4 | 1.90 |
| VGPPG | P.SGNAGPPGPPGP.A | GKE | 1004.48 | 14 | 6.67 |
| SGPAG | P.TGARGAPGDRGEPGPPGP.A | GFA | 1645.805 | 13 | 6.19 |
| TGDAG | P.VGPPGPPGPP.G | PPG | 671.468 | 3 | 1.43 |
| TGDAG | P.VGPPGPPGPPGPPGPP. | | 1373.722 | 19 | 9.05 |
| KGEPG | P.VGVQGPPGP.A | GEE | 807.437 | 3 | 1.43 |
| GDAGP | V.GPPGPPGPP.G | PPG | 772.399 | 2 | 0.95 |

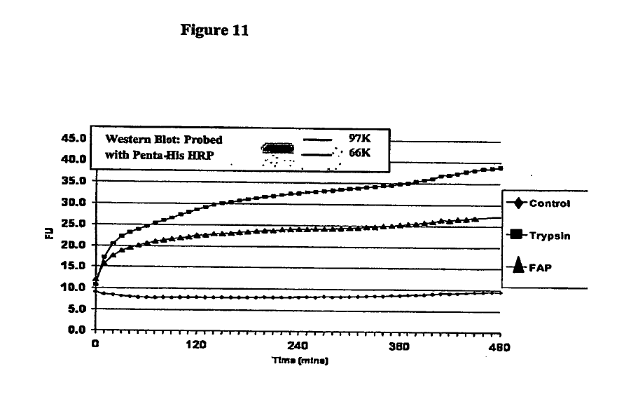

FIG. 11 shows the fluorescence quenched Collagen I labeled with the fluorophore FITC was incubated with purified FAP or Trypsin as positive control. Protein hydrolysis releases FITC labeled peptide fragments resulting in increased fluorescence intensity over time. Inset shows Western blot analysis demonstrating single band of His-tagged FAP after Ni-resin purification.

### Stability of Fluorescence Quenched Peptide Substrates in Human Plasma

We evaluated the stability of the VGP//AGK substrate by incubated in human plasma for ˜1 hr. These studies demonstrated that this FAP substrate was stable to cleavage in human plasma at a concentration of 50 μM (e.g. ˜Kₘ). This suggests that prodrugs generated by coupling the TG analog to selected FAP substrates will most likely be stable to non-specific hydrolysis in plasma. The results suggest also that insignificant amounts of enzymatically active soluble FAP must be present in normal human plasma as no significant hydrolysis of a highly active FAP substrate (e.g., VGPAGK) was observed.

Mapping FAP Cleavage Sites in Full Length Recombinant Gelatin from Human Collagen I

Once the methodologies for analyzing the cleavage fragment were worked out using the 8.5 kDa gelatin fragment, we proceeded to analyze the complete map of FAP cleavage sites within the 100 kDa gelatin produced by FibroGen, FIG. 14.

Ranking the cleavage products by normalized ion current revealed a strong preference for cleavage after the GP dipeptide motif, FIG. 14. Positional analysis of amino acids in positions P7-P′1 was performed, FIG. 11. Based on amino acids observed in each position as percent of the total, the overall consensus amino acid sequence was PPGPPGPA, FIG. 14. This peptide would not be optimal for incorporation into the FAP prodrug due to its significant hydrophobicity. However, further positional analysis demonstrated preferences for Asp or Glu residues in P7, Arg in P6, Ala, Asp or Glu in P4, Ser or Thr in P3and Ala in P′1 to produce a second consensus peptide of (D/E)RG(E/A)(T/S)GPA. In addition, sequences based on DRGETGPA (Red text in figure) are frequently found in the cleavage map, FIG. 14. FIG. 14 shows the complete map of FAP cleavage sites within 100 kDa recombinant human gelatin prepared from human collagen I.

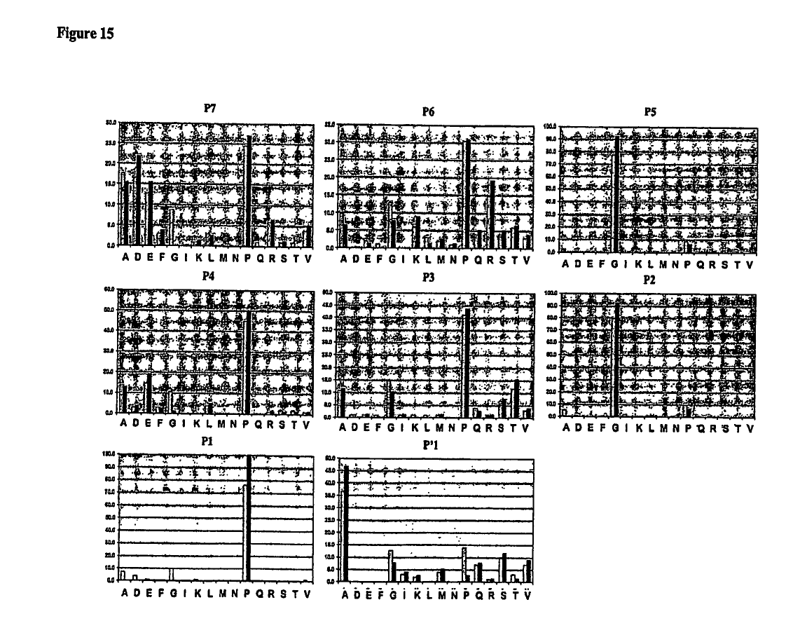

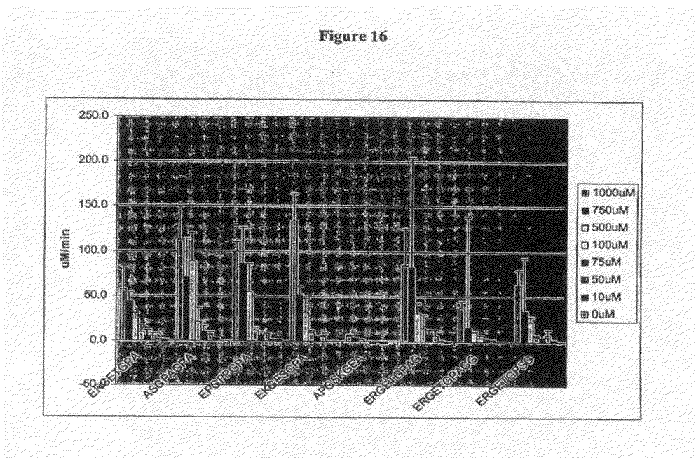

FIG. 15 shows the positional analysis of amino acids from FAP cleavage sites within 100 kDa recombinant human gelatin. (Blue column represents percent of each amino acid in positions P7-P′1 for all cleavage sites; Purple column indicates percent of each amino acid in positions P7-P′1 in only those sequences having Proline at cleavage site in the P1 position. FIG. 16 shows FAP hydrolysis over a range of concentrations of a series of fluorescence quenched peptides selected based on the 100 kDa gelatin cleavage map

### Production of FAP Positive Cancer Cell Lines

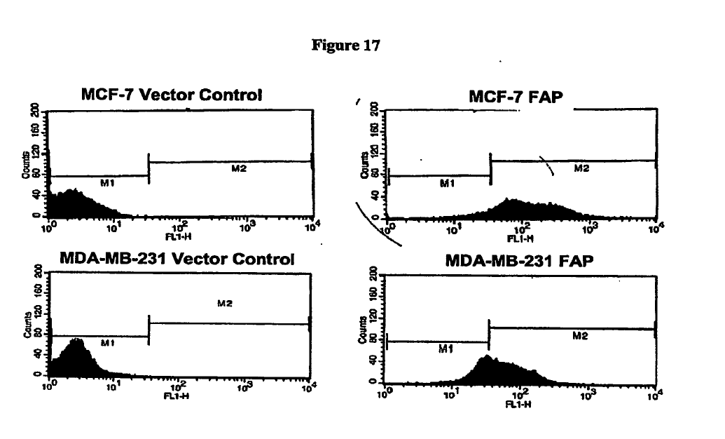

Human breast cancer cell lines MCF-7 and MDA-MB-231 were selected for transfection because the cells were negative for FAP when grown under standard culture conditions (25). Details of transfection methodology are described in methods section of Specific Aim 2 below. Briefly, a 2.2 kb PCR fragment was purified by gel electrophoresis, digested with Nhe1 and cloned into bicistronic pIRES vector (kindly provided by Dr. Ben Park, The Johns Hopkins University) previously digested with Nhe 1. Final construct was designated as pFAPIRES. Sequencing primers were designed for the entire length of the gene and were confirmed to be correct. The cells were transfected using Fugene 6 (Roche). Control transfections were done on the same lines with the empty IRES vector. Cells were characterized for production of FAP by Flow Cytometry (FIG. 17). FIG. 17 shows the flow cytometric traces of individual FAP-transfected and empty vector transfected controls demonstrating positive expression of FAP in both cell lines.

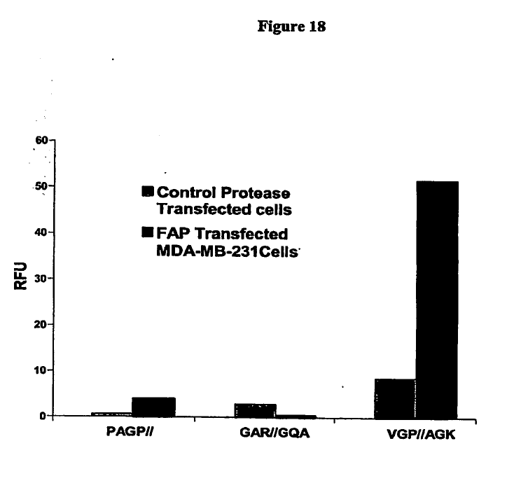

Subsequently, one of the MDA-MB-231 clones was used to evaluate FAP activity in vitro. In this assay cells were grown in serum containing media to 70% confluency followed by addition of fluorescence quenched peptides into the media at concentration of 50 μM. As a control cell lines transfected with the carboxypeptidase PSMA using the same vector system were used. Cells were incubated with three different FAP substrates, FIG. 18. Only one of the substrates, VGP//AGK was appreciably hydrolyzed by FAP-positive cells. This peptide was relatively stable to hydrolysis by control media suggesting that significant prolyl hydrolase activity may not be present in the media from these cells. FIG. 18 shows the hydrolysis of fluorescently quenched FAP peptide substrates in conditioned media from FAP-transfected MDA-MB-231 cells and control cells transfected with PSMA

1. A peptide comprising an amino acid sequence having a cleavage site specific for an enzyme having a proteolytic activity of fibroblast activation protein (FAP), wherein the peptide comprises the sequence of any one of SEQ ID NO. 1-44 and peptide sequences listed in Tables 1, 2, and 3 and FIGS. 12 and 14.

2. The peptide of claim 1, further comprising a nitrotyrosine quencher at the amino terminus of the peptide.

3. The peptide of claim 1, further comprising a capping group attached to the N-terminus of the peptide, wherein the capping group inhibits endopeptidase activity on the peptide.

4. The peptide of claim 3, wherein the capping comprises one or more of acetyl, morpholinocarbonyl, benzyloxycarbonyl, glutaryl or succinyl substituents.

5. The peptide of claim 1, further comprising an added substituent that renders the peptide water-soluble.

6. The peptide of claim 5, wherein the added substituent is a polymer.

7. The peptide of claim 6, wherein the polymer is selected from the group consisting of polylysine, polyethylene glycol (PEG), and a polysaccharide.

8. The peptide of claim 7, wherein the polysaccharide is selected from the group consisting of modified or unmodified dextran, cyclodextrin, and starch.

9. The peptide of claim 1, further comprising one or more of an antibody or a peptide toxin attached to the carboxy terminus of the peptide.

10. The peptide of claim 1, further comprising a peptide toxin attached to the peptide.

11-13. (canceled)

14. A peptide composition comprising a plurality of peptides, each peptide comprising an amino acid sequence having a cleavage site specific for an enzyme having a proteolytic activity of FAP (FAP), wherein each peptide comprises (D/E)RG(E/A)(T/S)GPA or peptide sequences with Proline in P1 but having either nothing in P′1, Ala, Ser, Val in P′1, or Ala, Ser Val in P′1 and Gly in P′2.

15. A polynucleotide encoding the peptide comprising an amino acid sequence having a cleavage site specific for an enzyme having a proteolytic activity of fibroblast activation protein (FAP), wherein the peptide comprises the sequence of any one of SEQ ID NO. 1-44 and peptide sequences listed in Tables 1, 2, and 3 and FIGS. 12 and 14.

16. A composition comprising a prodrug, the prodrug comprising
a therapeutically active drug; and
a peptide comprising an amino acid sequence having a cleavage site specific for an enzyme having a proteolytic activity FAP,
wherein the peptide is 20 or fewer amino acids in length, and wherein the peptide is linked to the therapeutically active drug to inhibit the therapeutic activity of the drug, and wherein the therapeutically active drug is cleaved from the peptide upon proteolysis by an enzyme having a proteolytic activity of FAP.

17. The composition of claim 16, wherein the peptide is linked directly to the therapeutic drug.

18-21. (canceled)

22. The composition of claim 16, wherein the therapeutically active drug is an anthracycline, a taxane, a vinca alkaloid, an antiandrogen, an antifolate, a nucleoside analog, a topoisomerase inhibitor, an alkylating agent, a primary amine containing thapsigargins and thapsigargin derivatives or a targeted radiation sensitizer.

23. The composition of claim 22, wherein the anthracycline is selected from the group consisting of doxorubicin, daunorubicin, epirubicin, and idarubicin.

24-35. (canceled)

36. A method of producing a prodrug, the method comprising the step of linking a therapeutically active drug and a peptide comprising an amino acid sequence having a cleavage site specific for an enzyme having a proteolytic activity FAP, wherein the peptide is 20 or fewer amino acids in length, and wherein the peptide is linked to the therapeutically active drug to inhibit the therapeutic activity of the drug, and wherein the therapeutically active drug is cleaved from the peptide upon proteolysis by an enzyme having a proteolytic activity of FAP.

37-41. (canceled)

42. A method of treating a FAP related disorder, comprising administering the composition of claim 22 in a therapeutically effective amount to a subject having the cell proliferative disorder.

43-49. (canceled)

50. A method of detecting FAP-producing tissue comprising: contacting the tissue with a composition comprising a detectably labeled peptide of claim 1 for a period of time sufficient to allow cleavage of the peptide; and detecting the detectable label.

51-56. (canceled)

57. A method of selecting a fibroblast activation protein (FAP) activatable prodrug wherein the prodrug is substantially specific for target tissue comprising FAP-producing cells, comprising: a) contacting cells of a target tissue with a candidate prodrug composition with; b) contacting non-target tissue with the prodrug composition; and c) selecting a candidate prodrug composition that is substantially toxic towards target tissue cells, and not substantially toxic towards non-target tissue cells.

58. A method of determining the activity of FAP in a comprising: a) contacting the sample with a composition comprising a detectably labeled peptide of any one of claim 1 for a period of time sufficient to allow cleavage of the peptide; b) detecting the detectable label; c) comparing a detection level with a standard.

59. A method of imaging FAP-producing tissue, the method comprising: a) administering a peptide of claim 1 linked to a lipophilic imaging label to a subject having or suspected of having an FAP producing associated cell-proliferative disorder; b) allowing a sufficient period of time to pass to allow cleavage of the peptide by FAP and to allow clearance of uncleaved peptide from the subject to provide a reliable imaging of the imaging label; and c) imaging the subject.

60. (canceled)

61. A recombinant polynuclietide encoding a peptide comprising an amino acid sequence having a cleavage site specific for an enzyme having a proteolytic activity of fibroblast activation protein (FAP), wherein the peptide comprises the sequence of any one of SEQ ID NO. 1-44 and peptide sequences listed in Tables 1, 2, and 3 and FIGS. 12 and 14.

62. A cell transformed with a recombinant polynucleotide of claim 61.

63. A transgenic organism comprising a recombinant polynucleotide of claim 61.

64. A method of producing a polypeptide of claim 1, the method comprising: a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide comprising an amino acid sequence having a cleavage site specific for an enzyme having a proteolytic activity of fibroblast activation protein (FAP), wherein the peptide comprises the sequence of any one of SEQ ID NO. 1-44 and peptide sequences listed in Tables 1, 2, and 3 and FIGS. 12 and 14, and b) recovering the polypeptide.
